(19) United States
(12) Patent Application Publication
(10) Pub. No.: US 2026/0027100 A1
(43) Pub. Date: Jan. 29, 2026

(54) THERAPEUTIC TYROSINE KINASE INHIBITORS FOR MYELIN OLIGODENDROCYTE GLYCOPROTEIN ANTIBODY DISEASE (MOGAD)

(71) Applicant: Principia Biopharma Inc., Bridgewater, NJ (US)

(72) Inventors: Timothy J. Turner, Belmont, MA (US); Erik Wallstroem, Cambridge, MA (US)

(73) Assignee: Principia Biopharma Inc., Bridgewater, NJ (US)

(21) Appl. No.: 18/864,343

(22) PCT Filed: May 12, 2023
(86) PCT No.: PCT/US2023/022042

§ 371 Date: Nov. 8, 2024

Related U.S. Application Data
(60) Provisional application No. 63/341,486, filed on May 13, 2022.
(60) Provisional application No. 63/433,853, filed on Dec. 20, 2022.

(51) Int. Cl. A61K 31/4545

(52) U.S. Cl. CPC A61K 31/4545

(57) ABSTRACT

This disclosure relates to the field of therapeutic tyrosine kinase inhibitors, in particular, Bruton tyrosine kinase (“BTK”) inhibitors, for treatment of subjects with myelin oligodendrocyte glycoprotein antibody disease (MOGAD).

### INTRODUCTION

This disclosure relates to the field of therapeutic tyrosine kinase inhibitors, in particular, Bruton tyrosine kinase (“BTK”) inhibitors, for treating Myelin Oligodendrocyte Glycoprotein Antibody Disease (MOGAD).

Myelin oligodendrocyte glycoprotein (MOG) is a highly conserved protein that is exclusively expressed in oligodendrocytes in the central nervous system (CNS). MOG is located on the surface of myelin sheaths in the central nervous system (Fan et al. Mult Scler Relat Disord. 2018, 20, 144-152; Kezuka et al. Jpn J Ophthalmol. 2018, 62, 101-108). Multiple isoforms of MOG exist that have identical extracellular immunoglobulin (Ig) domains, but differentially spliced intracellular C-termini. The differences in the C-terminal amino acids are the basis to distinguish a or R isoforms of MOG. While the function of MOG is not exactly known, it is a target of the immune system in MOGAD (dos Passos et al. Front. Neurol. 2018, 9, 217). MOG-IgG antibodies (MOG-IgG) have been extensively studied in the last two decades in different acquired demyelinating syndromes (ADSs). The development and use of highly specific cell-based assays enabled the description of a variety of clinical disease manifestations ranging from certain ADSs, for example, acute disseminated encephalomyelitis (ADEM) predominantly in children or optic neuritis mostly in adults, to cases of encephalitis with seizures. This broad spectrum of clinical phenotypes associated with MOG-IgG has evolved into a new inflammatory CNS disease entity that is distinct from both multiple sclerosis (MS) and neuromyelitis optica spectrum disorders (NMOSDs).

MOG antibody disease (MOGAD) is a recently characterized neuro-inflammatory condition that preferentially causes inflammation in the optic nerve, but it can also cause inflammation in the spinal cord and brain. Diagnosis is confirmed when MOG antibodies in the blood are found in patients who have repeated inflammatory attacks of the central nervous system (Weber et al. Ther Adv Neurol Disord. 2018, 11, 1-15). Those with MOGAD may previously have been diagnosed with Neuromyelitis Optica Spectrum Disorder (NMOSD), Transverse Myelitis (TM), Acute Disseminated Encephalomyelitis (ADEM), Optic Neuritis (ON), or multiple sclerosis (MS) because of the pattern of inflammation it causes including brain, spinal cord and optic nerve damage. Patients with persistently positive antibodies are at risk for recurrent events. Those with MOGAD do not test positive for the NMO antibody called aquaporin 4 (AQP-4). MOGAD and AQP-4 positive NMOSD are thought to have distinct immunological mechanisms. Furthermore, those with MOGAD seem to be less likely to have other autoimmune disorders (such as rheumatoid arthritis, Hashimoto's thyroiditis, etc.) than those with AQP-4 positive NMOSD.

There are currently approximately 4,000 diagnosed patients yearly in the US and EU, but it is believed that MOGAD is underdiagnosed on the order of about 10%, i.e., MOGAD is estimated to be prevalent in about 40,000 patients/year in US/EU.

MOGAD presents itself with symptoms including loss of color vision, loss or blurring of vision, paralysis/paraparesis of limbs, and loss of bladder/bowel control. This condition presents a number of unmet needs, including that MOGAD patients are often misdiagnosed and difficult to treat; there are currently no treatments specifically approved or in development for MOGAD, and as a result the current standard of care treatment is the intravenous administration of immunosuppressants like methylprednisolone or oral prednisolone. There therefore exists an unmet need for effective therapies for MOGAD.

The Bruton's tyrosine kinase (BTK) pathway is critical to signaling in B lymphocytes and myeloid cells including CNS microglia. Each of these cell types has been implicated in the pathophysiology of multiple sclerosis (MS). Further, as BTK signaling is vital for maturation of B cells into antibody-secreting plasma cells, BTK inhibition can modulate both cellular and humoral immunity. Accordingly, compounds that inhibit BTK that are able to both inhibit antigen-induced B-cell activation responsible for neuroinflammation and modulate maladaptive microglial cells linked to neuroinflammation in the brain and spinal cord may be useful in treating MOGAD with superior benefits compared to the paucity of available therapies currently available. BTK inhibitors may modify MOGAD disease progression through two distinct mechanisms, 1) modulation of B cell mediated autoinflammatory processes, and 2) inhibition of Fc-receptor mediated microglial inflammatory demyelination.

Drug-induced liver injury has been identified in the ongoing tolebrutinib Phase 3 trials. The reported events occurred between months 2 to 3 after the start of tolebrutinib administration, and the elevation of liver enzymes appears reversible after tolebrutinib discontinuation. Thus, there is a need to mitigate the risk of hepatic injury and provide safe treatment for patients with MOGAD.

### SUMMARY

The present disclosure relates to methods of treating myelin oligodendrocyte glycoprotein antibody disease (MOGAD) comprising administering to a subject in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one or a pharmaceutically acceptable salt thereof.

The present disclosure also relates to methods of reducing the frequency of MOGAD relapse in a subject having MOGAD comprising administering to a subject in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one or a pharmaceutically acceptable salt thereof.

The present disclosure further relates to methods of reducing MOG antibody titers in a subject having MOGAD comprising administering to a subject in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one or a pharmaceutically acceptable salt thereof.

In some embodiments, the subject has at least one symptom of MOGAD prior to treatment.

In some embodiments, the at least one MOGAD symptom is chosen from loss of color vision, loss or blurring of vision, paralysis/paraparesis of limbs, and loss of bladder/bowel control.

In some embodiments, the treatment reduces or eliminates at least one symptom of MOGAD.

In some embodiments, the subject is relapse-free about 1 year.

In some embodiments, the subject has at least one confirmed relapse in the previous 12 months or 2 confirmed relapses in the previous 24 months at screening.

In some embodiments, the subject in relapse is administered a corticosteroid.

In some embodiments, the corticosteroid is methylprednisone.

In some embodiments, a dose of about 5 mg to about 60 mg of the BTK inhibitor is administered.

In some embodiments, the dose is 5 mg.

In some embodiments, the dose is 15 mg.

In some embodiments, the dose is 30 mg.

In some embodiments, the dose is 60 mg.

In some embodiments, the dose is once daily.

In some embodiments, the dose is administered once daily with food.

In some embodiments, the dose is 60 mg and is administered once daily with food.

In some embodiments, the BTK inhibitor compound is administered as monotherapy.

In some embodiments, MOGAD is chosen from acquired demyelinating syndromes (ADS).

In some embodiments, the ADS is acute disseminated encephalomyelitis (ADEM).

In some embodiments, the subject is a human.

In some embodiments, the subject is a human subject ranging in age from 12 to 55 years old.

The present disclosure also relates to a BTK inhibitor comprising (R)-1-(1-acryloylpiperidin-3-yl)-4-amino-3-(4-phenoxyphenyl)-1H-imidazo[4,5-c]pyridin-2(3H)-one for use in a method for treating MOGAD in a subject in need thereof.

The present disclosure further relates to a BTK inhibitor comprising (R)-1-(1-acryloylpiperidin-3-yl)-4-amino-3-(4-phenoxyphenyl)-1H-imidazo[4,5-c]pyridin-2(3H)-one for use in a method for reducing the frequency of MOGAD relapse in a subject having MOGAD in need thereof.

The present disclosure further relates to a BTK inhibitor comprising (R)-1-(1-acryloylpiperidin-3-yl)-4-amino-3-(4-phenoxyphenyl)-1H-imidazo[4,5-c]pyridin-2(3H)-one for use in a method of reducing or eliminating MOG antibody titers in a subject having MOGAD in need thereof.

The present disclosure also relates to methods of treating MOGAD, comprising the steps of:
(a) performing an iron panel test in a patient's blood or serum;
(b) detecting levels of the iron panel test that are within normal ranges; and
(c) administering a therapeutically effective amount BTK inhibitor comprising (R)-1-(1-acryloylpiperidin-3-yl)-4-amino-3-(4-phenoxyphenyl)-1H-imidazo[4,5-c]pyridin-2(3H)-one to the patient,
   wherein the iron panel test measures any one or more of levels of iron, ferritin, transferrin saturation, and total iron-binding capacity (TIBC) in a patient's blood or serum and wherein the normal ranges of the iron panel test include one or more of (i) an iron level of 60 to 170 μg/dL, (ii) a ferritin level of ≤500 g/L (iii) a transferrin saturation level≤50% in a male patient or ≤40% in a female patient, and (iv) a TIBC of 240 to 450 μg/dL.

The present disclosure also relates to methods of treating MOGAD, comprising the steps of:
(a) detecting a level of transferrin saturation in a patient's blood or serum that is within normal range; and
(b) administering a therapeutically effective amount BTK inhibitor comprising (R)-1-(1-acryloylpiperidin-3-yl)-4-amino-3-(4-phenoxyphenyl)-1H-imidazo[4,5-c]pyridin-2(3H)-one to the patient,
   wherein the transferrin saturation level that is within normal range in the blood or serum of a male patient is a transferrin saturation of ≤50%, and the transferrin saturation level that is within normal range in the blood or serum of a female patient is a transferrin saturation of ≤40%.

The present disclosure also relates to methods of treating MOGAD, comprising the steps of:
(a) detecting a level of ferritin in a patient's blood or serum that is within normal range; and
(b) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient,
   wherein the ferritin level that is within normal range in the blood or serum of the patient is ≤500 g/L.

The present disclosure also relates to methods of treating MOGAD, comprising the steps of:
(a) performing liver function tests in a patient;
(b) detecting suitable liver function in the patient; and
(c) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient,
   wherein the liver function tests measure one or more of the levels of aspartate transaminase (AST), alanine transaminase (ALT), albumin, alkaline phosphatase, total and direct bilirubin, and total protein in a patient's blood, and wherein the patient having a suitable liver function has one or more of ALT≤1.5× upper limit of normal (ULN), AST levels of ≤1.5×ULN, alkaline phosphatase≤2×ULN (unless caused by non-liver related disorder or explained by a stable chronic liver disorder) and total bilirubin≤1.5×ULN (unless due to Gilbert syndrome or non-liver-related disorder).

The present disclosure also relates to methods of treating MOGAD, comprising the steps of:
(a) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one (Compound) to a patient in need thereof,
(b) measuring the level of alanine aminotransferase (ALT) in the patient;
(c) detecting a level of ALT of >8× upper limit of normal (ULN);
(d) ceasing administration of the Compound to the patient; and optionally
(e) monitoring the level of ALT in the patient; and
(f) resuming administration of a therapeutically effective amount of the Compound to the patient when the patient's level of ALT is determined to be <1.5×ULN.

The present disclosure also relates to methods of treating MOGAD, comprising the steps of:
(a) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one (Compound) to a patient in need thereof,
(b) measuring the level of alanine aminotransferase (ALT) in the patient;
(c) detecting a level of ALT of >5× upper limit of normal (ULN) during a period of at least two weeks;
(d) ceasing administration of the Compound to the patient; and optionally
(e) monitoring the level of ALT in the patient; and
(f) resuming administration of a therapeutically effective amount of the Compound to the patient when the patient's level of ALT is determined to be <1.5×ULN.

The present disclosure also relates to methods of treating MOGAD, comprising the steps of:
(a) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one (Compound) to a patient in need thereof,
(b) measuring the level of alanine aminotransferase (ALT) in the patient;
(c) detecting a level of ALT of >3× upper limit of normal (ULN);
(d) measuring one or more of total bilirubin and international normalized ratio (INR) in a patient;
(e) detecting one or more of total bilirubin>2×ULN and INR>1.5;
(f) ceasing administration of the Compound to the patient; and optionally
(g) monitoring the level of ALT in the patient; and
(h) resuming administration of a therapeutically effective amount of the Compound to the patient when the patient's level of ALT is determined to be <1.5×ULN.

The present disclosure also relates to methods of treating MOGAD, comprising the steps of:
(a) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one (Compound) to a patient in need thereof,
(b) measuring the level of alanine aminotransferase (ALT) in the patient;
(c) detecting a level of ALT of >3× upper limit of normal (ULN);
(d) ceasing administration of the Compound to the patient if the patient experiences one or more of fatigue, nausea, vomiting, right upper quadrant pain or tenderness, fever, rash, and eosinophilia>5%; and optionally
(e) monitoring the level of ALT in the patient; and
(f) resuming administration of a therapeutically effective amount of the Compound to the patient when the patient's level of ALT is determined to be <1.5×ULN.

In some embodiments, the level of ALT in step (b) is determined at least monthly.

In some embodiments, the level of ALT in step (d) is monitored at least weekly.

In some embodiments, the level of ALT in step (d) is monitored every 2 to 3 days.

The present disclosure also relates to methods of treating MOGAD in a patient in need thereof, comprising administering a therapeutically effective amount BTK inhibitor comprising (R)-1-(1-acryloylpiperidin-3-yl)-4-amino-3-(4-phenoxyphenyl)-1H-imidazo[4,5-c]pyridin-2(3H)-one to the patient, wherein the patient is not receiving potent and moderate inducers of cytochrome P450 3A (CYP3A) or potent inhibitors of CYP2C8 hepatic enzymes.

The present disclosure also relates to methods of treating MOGAD in a patient in need thereof, comprising the steps of.
(a) advising the patient to limit alcohol consumption during treatment; and
(b) administering a therapeutically effective amount BTK inhibitor comprising (R)-1-(1-acryloylpiperidin-3-yl)-4-amino-3-(4-phenoxyphenyl)-1H-imidazo[4,5-c]pyridin-2(3H)-one to the patient, wherein the patient is female and is advised to limit alcohol consumption to 14 grams/day or less, or the patient is male and is advised to limit alcohol consumption to 28 grams/day or less.

### BRIEF DESCRIPTION OF DRAWINGS

FIG. 1A provides the study design of Example 1.

FIG. 1B provides the study design of Example 2.

FIG. 2 provides suggested actions and follow-up assessments in the event of neutropenia.

FIG. 3 provides suggested actions and follow-up assessments in the event of thrombocytopenia.

FIG. 4A provides suggested actions and follow-up assessments in the event of increased alanine aminotransferase (ALT) algorithm for Example 1.

FIG. 4B provides suggested actions and follow-up assessments in the event of increased alanine aminotransferase (ALT) algorithm for Example 2.

FIG. 5 provides suggested actions and follow-up assessments in the event of increased in serum creatinine.

FIG. 6 provides suggested actions and follow-up assessments when progressive multifocal leukoencephalopathy (PML) is suspected.

FIG. 7 provides a description of the expanded disability status scale score (EDSS) in view of the level of disability.

FIG. 8 provides suggested actions and follow-up assessments in the event of increase in CPK of non-cardiac origin and not related to intensive physical activity.

### DETAILED DESCRIPTION

Reference will now be made in detail to certain embodiments, examples of which are illustrated in the accompanying drawings. While the disclosure provides illustrated embodiments, it will be understood that they are not intended to limit the invention to those embodiments. On the contrary, the invention is intended to cover all alternatives, modifications, and equivalents, which may be included within the disclosure as defined by the appended claims.

The section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. DEFINITIONS

Unless otherwise stated, the following terms used in the specification and claims are defined for the purposes of this disclosure and have the following meaning.

As used herein, “the BTK inhibitor,” “the BTK inhibitor compound,” and “the compound”, refers to (R)-1-(1-acryloylpiperidin-3-yl)-4-amino-3-(4-phenoxyphenyl)-1H-imidazo[4,5-c]pyridin-2(3H)-one having the following structure:

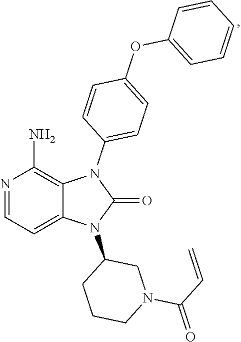 [SMILES: C=CC(=O)N1CCC[C@@H](n2c(=O)n(-c3ccc(Oc4ccccc4)cc3)c3c(N)nccc32)C1]

which is also known as “tolebrutinib,” and 4-amino-3-(4-phenoxyphenyl)-1-[(3R)-1-(prop-2-enoyl)piperidin-3-yl]-1,3-dihydro-2H-imidazo[4,5-c]pyridin-2-one having the following structure:

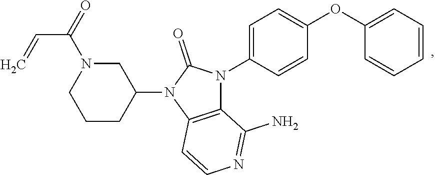 [SMILES: C=CC(=O)N1CCCC(n2c(=O)n(-c3ccc(Oc4ccccc4)cc3)c3c(N)nccc32)C1]

or a pharmaceutically acceptable salt thereof.

A “pharmaceutically acceptable carrier” or a “pharmaceutically acceptable excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Treating” or “treatment” of a disease includes:
(1) inhibiting the disease, e.g., arresting or reducing the development of the disease or its clinical symptoms; or
(2) relieving the disease, e.g., causing regression of the disease or its clinical symptoms.

“Optional” or “optionally” means that the subsequently described event or circumstance may but need not occur, and that the description includes instances where the event or circumstance occurs and instances in which it does not.

A “therapeutically effective amount” means the amount of the BTK inhibitor compound, that, when administered to a mammal for treating a disease, is sufficient to affect such treatment for the disease. The “therapeutically effective amount” will vary depending on the compound, the disease and its severity and the age, weight, etc., of the mammal to be treated.

“Relapse of MOGAD” is defined as an acute or subacute onset of, new neurological symptoms or worsening of previous neurological symptoms with an objective change on neurological examination.

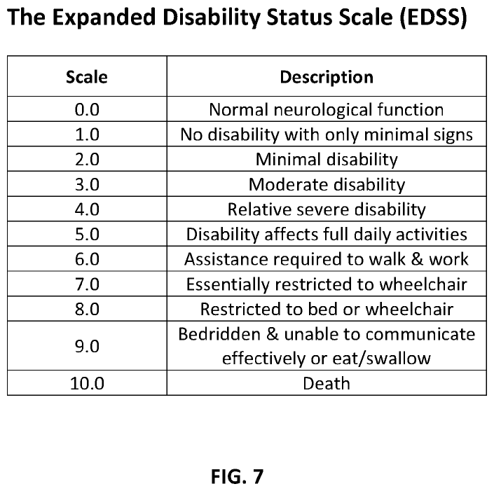

“Expanded disability status scale (EDSS) score” is a method of quantifying disability in multiple sclerosis and monitoring changes in the level of disability over time. The EDSS scale ranges from 0 to 10 in 0.5 unit increments that represent higher levels of disability. EDSS steps 1.0 to 4.5 refer to people with MS who are able to walk without any aid and is based on measures of impairment in eight functional systems (FS): pyramidal—muscle weakness or difficulty moving limbs; cerebellar—ataxia, loss of balance, coordination or tremor; brainstem—problems with speech, swallowing and nystagmus; sensory—numbness or loss of sensations; bowel and bladder function; visual function—problems with sight; cerebral functions—problems with thinking and memory. EDSS steps 5.0 to 9.5 are defined by the impairment to walking. See, e.g., FIG. 7. Information about this score is found at Kurtzke et al. Neurology 1983, 33, 1444-1452.

“Negative serum aquaporin-4 antibody titer” means subjects that have tested negative against aquaporin-4 receptor antibody in their serum, which is used for diagnosis and evaluation of neuromyelitis optica, acute myelitis, spinal cord lesions, autoimmune encephalitis, or neuromyelitis optica spectrum (NMO) disorders. “Aquaporin-4 receptor antibody” is a highly specific serum marker for neuromyelitis optica that can facilitate the differential diagnosis of the diseases stated above. If the patient tests positive for the aquaporin-4 receptor antibody in their serum, then the patient has NMO and not MOGAD.

Before describing the present teachings in detail, it is to be understood that the disclosure is not limited to specific compositions or process steps, as such may vary.

It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims.)

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Or” is used in the inclusive sense, i.e., equivalent to “or,” unless the context requires otherwise.

“Ceasing” or “cessation” when used regarding administration of a BTK inhibitor compound means that a BTK inhibitor compound is no longer being administered to the patient on either a temporary or permanent basis.

“Monitoring” with reference to assessment of the level of ALT in a patient means checking, and/or detecting the level of ALT in a patient over at least two points in time; in some embodiments, over a period of time; in some embodiments, monthly; in some embodiments, at least monthly; in some embodiments, weekly; in some embodiments, at least weekly; in some embodiments, every 5 days; in some embodiments, every 3 days; in some embodiments, every 2 to 3 days; in some embodiments, every 2 days; in some embodiments, daily.

### II. ADMINISTERED BTK INHIBITOR COMPOUND

In some embodiments, a BTK inhibitor compound, (R)-1-(1-acryloylpiperidin-3-yl)-4-amino-3-(4-phenoxyphenyl)-1H-imidazo[4,5-c]pyridin-2(3H)-one is administered for treating myelin oligodendrocyte glycoprotein antibody disease (MOGAD) in a subject in need thereof. In some embodiments, the BTK inhibitor compound is a pharmaceutically acceptable salt of (R)-1-(1-acryloylpiperidin-3-yl)-4-amino-3-(4-phenoxyphenyl)-1H-imidazo[4,5-c]pyridin-2(3H)-one. In some embodiments, a therapeutically effective amount of the BTK inhibitor compound is administered. In some embodiments, a dose of 5 to 60 mg of the BTK inhibitor compound is administered. In some embodiments, a dose of 60 mg of the BTK inhibitor compound is administered. In some embodiments, a dose of 60 mg once daily of the BTK inhibitor compound is administered.

In some embodiments, a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one is provided for use in a method for treating MOGAD in a subject in need thereof.

In some embodiments, a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one is provided for use in a method for reducing the frequency of MOGAD relapse in a subject having MOGAD in need thereof.

In some embodiments, a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one is provided for use in reducing MOG antibody titers in a subject having MOGAD in need thereof.

The BTK inhibitor compound can be prepared according to the methods and schemes described in, e.g., U.S. Pat. No. 9,688,676 B2, in particular the content of column 62, line 8 to column 65 line 32, and column 67, line 28 to column 69, which is incorporated herein by reference.

The following preparation of the compound of (R)-1-(1-acryloylpiperidin-3-yl)-4-amino-3-(4-phenoxyphenyl)-1H-imidazo[4,5-c]pyridin-2(3H)-one, is given to enable those skilled in the art to prepare the BTK inhibitor compound. The synthetic route should not be considered as limiting the scope of the disclosure, but merely as being illustrative and representative thereof.

Exemplary synthesis of (R)-1-(1-acryloylpiperidin-3-yl)-4-amino-3-(4-phenoxyphenyl)-1H-imidazo[4,5-c]pyridin-2(3H)-one:

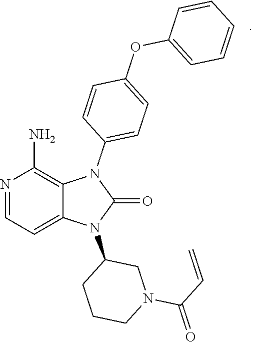 [SMILES: C=CC(=O)N1CCC[C@@H](n2c(=O)n(-c3ccc(Oc4ccccc4)cc3)c3c(N)nccc32)C1]

Into a 100 mL round-bottom flask was placed (R)-4-amino-3-(4-phenoxyphenyl)-1-(piperidin-3-yl)-1H-imidazo[4,5-c]pyridin-2(3H)-one (150 mg, 0.37 mmol, 1.00 equiv), DCM-CH₃OH (6 mL), TEA (113 mg, 1.12 mmol, 3.00 equiv). This was followed by the addition of prop-2-enoyl chloride (40.1 mg, 0.44 mmol, 1.20 equiv) dropwise with stirring at 0° C. in 5 min. The resulting solution was stirred for 2 h at 0° C. The resulting mixture was concentrated under vacuum. The residue was applied onto a silica gel column with dichloromethane/methanol (30:1). The crude product (100 mg) was purified by Prep-HPLC under the following conditions (Column, XBridge Prep C₁₈ OBD Column, 5 μm, 19*150 mm; mobile phase, water with 0.05% TFA and ACN (25.0% ACN up to 45.0% in 8 min). 54.5 mg product of (R)-1-(1-acryloylpiperidin-3-yl)-4-amino-3-(4-phenoxyphenyl)-1H-imidazo[4,5-c]pyridin-2(3H)-one was obtained as a white solid. LC-MS m/z: 465.2 (M+1).

### III. THERAPEUTIC METHODS

Provided herein are methods of treating myelin oligodendrocyte glycoprotein antibody disease (MOGAD) comprising administering to a subject in need thereof a therapeutically effective amount of the BTK inhibitor compound comprising (R)-1-(1-acryloylpiperidin-3-yl)-4-amino-3-(4-phenoxyphenyl)-1H-imidazo[4,5-c]pyridin-2(3H)-one, or a pharmaceutically acceptable salt thereof.

In some embodiments, the subject has at least one symptom of MOGAD prior to or during treatment. In some embodiments, the at least one MOGAD symptom is chosen from loss of color vision, loss or blurring of vision, paralysis/paraparesis of limbs, and loss of bladder/bowel control. In some embodiments, the treatment reduces or eliminates the at least one symptom. In some embodiments, the reduction or elimination of at least one symptom occurs after daily treatment for 1, 2, 3, 4, 5, 6, 7, 8, 9, 12, 11, or 12 weeks, or after 6 months, 1 year, 1.5 years, or 2 years.

In some embodiments, the subject is relapse free about 1 year. In some embodiments, the subject has had at least one confirmed relapse in the previous 12 months or 2 confirmed relapses in the previous 24 months at screening. In some embodiments, the subject in relapse is administered a corticosteroid. In some embodiments, the corticosteroid is methylprednisone.

In some embodiments, after about one year of treatment, the rate of increase of the expanded disability status scale score (EDSS) is reduced. In some embodiments, after about one year of treatment, the EDSS score is the same as or reduced from baseline. In some embodiments, the subject has an EDSS score of greater than 7.0 prior to treatment. In some embodiments, the subject has a negative serum aquaporin-4 antibody titer based on a cell-based assay. In some embodiments, the subject has a documented history of at least two confirmed MOGAD attacks.

In some embodiments the therapeutically effective amount is about 5 to about 60 mg. In some embodiments, the dose is 5 mg. In some embodiments, the dose is 15 mg. In some embodiments, the dose is 30 mg. In some embodiments, the dose is 60 mg.

In some embodiments, the dose is once daily. In some embodiments, the dose is administered once daily with food. In some embodiments, the dose is 60 mg and is administered once daily with food. In some embodiments, the BTK inhibitor compound is administered as monotherapy.

In some embodiments, the MOGAD is chosen from acquired demyelinating syndroms (ADS). In some embodiments, the ADS is acute disseminated encephalomyelitis (ADEM).

In some embodiments, the subject is a mammal. In some embodiments, the mammal is a human. In some embodiments, the subject is a human subject ranging in age from 12 to 55 years old.

Also provided herein are methods of reducing the frequency of MOGAD relapse in a subject having MOGAD, comprising administering to a subject in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one or a pharmaceutically acceptable salt thereof.

In some embodiments, the subject has at least one symptom of MOGAD prior to treatment. In some embodiments, the at least one MOGAD symptom is chosen from loss of color vision, loss or blurring of vision, paralysis/paraparesis of limbs, and loss of bladder/bowel control. In some embodiments, the subject has at least one symptom of MOGAD prior to treatment, wherein the treatment reduces or eliminates the one or more symptoms.

In some embodiments, the subject is relapse free about 1 year. In some embodiments, the subject has at least one confirmed relapse in the previous 12 months or 2 confirmed relapses in the previous 24 months at screening. In some embodiments, the subject relapses and is administered a corticosteroid. In some embodiments, the corticosteroid is methylprednisone.

In some embodiments, after about one year of treatment, the rate of increase of the expanded disability status scale score (EDSS) is reduced. In some embodiments, after about one year of treatment, the EDSS score is the same as or reduced from baseline. In some embodiments, the subject has an EDSS score of greater than 7.0 prior to treatment. In some embodiments, the subject has an EDSS score that is leveled without increase post-treatment. In some embodiments, the subject has an EDSS score that reduces to 9.5 or below post-treatment. In some embodiments, the subject has an EDSS score that reduces to 9.0 or below post-treatment. In some embodiments, the subject has an EDSS score that reduces to 8.5 or below post-treatment. In some embodiments, the subject has an EDSS score that reduces to 8.0 or below post-treatment. In some embodiments, the subject has an EDSS score that reduces to 7.5 or below post-treatment. In some embodiments, the subject has an EDSS score that reduces to 7.0 or below post-treatment. In some embodiments, the subject has an EDSS score that reduces to 6.5 or below post-treatment. In some embodiments, the subject has an EDSS score that reduces to 6.0 or below post-treatment. In some embodiments, the subject has an EDSS score that reduces to 5.5 or below post-treatment. In some embodiments, the subject has an EDSS score that reduces to 5.0 or below post-treatment. In some embodiments, the subject has an EDSS score that reduces to 4.5 or below post-treatment. In some embodiments, the subject has an EDSS score that reduces to 4.0 or below post-treatment. In some embodiments, the subject has an EDSS score that reduces to 3.5 or below post-treatment. In some embodiments, the subject has an EDSS score that reduces to 3.0 or below post-treatment. In some embodiments, the subject has an EDSS score that reduces to 2.5 or below post-treatment. In some embodiments, the subject has an EDSS score that reduces to 2.0 or below post-treatment. In some embodiments, the subject has an EDSS score that reduces to 1.5 or below post-treatment. In some embodiments, the subject has an EDSS score that reduces to 1.0 or below post-treatment. In some embodiments, the subject has an EDSS score that reduces to 0.5 or below post-treatment. In some embodiments, the subject has a negative serum aquaporin-4 antibody titer based on a cell-based assay. In some embodiments, the subject has a documented history of at least two confirmed MOG antibody disease attacks. In some embodiments, the BTK inhibitor compound is administered as a monotherapy.

In some embodiments, the MOGAD is chosen from acquired demyelinating syndroms (ADS). In some embodiments, the ADS is acute disseminated encephalomyelitis (ADEM).

In some embodiments the therapeutically effective amount is about 5 to about 60 mg. In some embodiments, the subject is a mammal. In some embodiments, the mammal is a human. In some embodiments, the subject is a human subject ranging in age from 12 to 55 years old.

Still further provided herein are methods of reducing or eliminating MOG antibody titers in a subject having MOGAD, comprising administering to a subject in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one or a pharmaceutically acceptable salt thereof.

In some embodiments, the subject has at least one symptom of MOGAD prior to treatment. In some embodiments, the at least one MOGAD symptom is chosen from loss of color vision, loss or blurring of vision, paralysis/paraparesis of limbs, and loss of bladder/bowel control. In some embodiments, the subject has at least one symptom of MOGAD prior to treatment, wherein the treatment reduces or eliminates the one or more symptoms.

In some embodiments, the subject is relapse free about 1 year. In some embodiments, the subject has at least one confirmed relapse in the previous 12 months or 2 confirmed relapses in the previous 24 months at screening. In some embodiments, the subject relapses and is administered a corticosteroid. In some embodiments, the corticosteroid is methylprednisone.

In some embodiments, after about one year of treatment, the rate of increase of the expanded disability status scale score (EDSS) is reduced. In some embodiments, the EDSS score is the same as or reduced from baseline after about one year of treatment. In some embodiments, the subject has an EDSS score of greater than 7.0 prior to treatment. In some embodiments, the subject has an EDSS score that is leveled without increase post-treatment. In some embodiments, the subject has an EDSS score that reduces to 9.5 or below post-treatment. In some embodiments, the subject has an EDSS score that reduces to 9.0 or below post-treatment. In some embodiments, the subject has an EDSS score that reduces to 8.5 or below post-treatment. In some embodiments, the subject has an EDSS score that reduces to 8.0 or below post-treatment. In some embodiments, the subject has an EDSS score that reduces to 7.5 or below post-treatment. In some embodiments, the subject has an EDSS score that reduces to 7.0 or below post-treatment. In some embodiments, the subject has an EDSS score that reduces to 6.5 or below post-treatment. In some embodiments, the subject has an EDSS score that reduces to 6.0 or below post-treatment. In some embodiments, the subject has an EDSS score that reduces to 5.5 or below post-treatment. In some embodiments, the subject has an EDSS score that reduces to 5.0 or below post-treatment. In some embodiments, the subject has an EDSS score that reduces to 4.5 or below post-treatment. In some embodiments, the subject has an EDSS score that reduces to 4.0 or below post-treatment. In some embodiments, the subject has an EDSS score that reduces to 3.5 or below post-treatment. In some embodiments, the subject has an EDSS score that reduces to 3.0 or below post-treatment. In some embodiments, the subject has an EDSS score that reduces to 2.5 or below post-treatment. In some embodiments, the subject has an EDSS score that reduces to 2.0 or below post-treatment. In some embodiments, the subject has an EDSS score that reduces to 1.5 or below post-treatment. In some embodiments, the subject has an EDSS score that reduces to 1.0 or below post-treatment. In some embodiments, the subject has an EDSS score that reduces to 0.5 or below post-treatment. In some embodiments, the subject has a negative serum aquaporin-4 antibody titer based on a cell-based assay. In some embodiments, the subject has a documented history of at least two confirmed MOG antibody disease attacks. In some embodiments, the BTK inhibitor compound is administered as a monotherapy.

In some embodiments, the MOGAD is chosen from acquired demyelinating syndroms (ADS). In some embodiments, the ADS is acute disseminated encephalomyelitis (ADEM).

In some embodiments the therapeutically effective amount is about 5 to about 60 mg. In some embodiments, the subject is a mammal. In some embodiments, the mammal is a human. In some embodiments, the subject is a human subject ranging in age from 12 to 55 years old. In some embodiments, the subject is a human patient. In some embodiments, the subject is a human patient ranging in age form 12 to 55 years old.

In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to reduce the frequency of MOGAD relapse to a subject that has one or more symptoms of MOGAD prior to treatment. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to reduce the frequency of MOGAD relapse and the treatment reduces or eliminates one or more symptoms. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to reduce the frequency of MOGAD relapse to a subject that suffers from loss of color vision, loss or blurring of vision, paralysis/paraparesis of limbs, and loss of bladder/bowel control caused by MOGAD. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to reduce the frequency of MOGAD relapse to a subject that is relapse free about 1 year. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to reduce the frequency of MOGAD relapse to a subject that has at least one confirmed relapse in the previous 12 months or 2 confirmed relapses in the previous 24 months at screening. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to reduce the frequency of MOGAD relapse to a subject that is administered a corticosteroid. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to reduce the frequency of MOGAD relapse to a subject that is administered methylprednisone. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one that is administered to reduce the frequency of MOGAD relapse, wherein after about one year of treatment, the rate of increase of the expanded disability status scale score (EDSS) is reduced. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one that is administered to reduce the frequency of MOGAD relapse, wherein after about one year of treatment, the EDSS score is the same as or reduced from baseline. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one that is administered to reduce the frequency of MOGAD relapse, wherein after about one year of treatment, the rate of increase of the expanded disability status scale score (EDSS) is reduced to a subject that has an EDSS score of greater than 7.0 prior to treatment. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one that is administered to reduce the frequency of MOGAD relapse, wherein after about one year of treatment, the EDSS score is the same as or reduced from baseline to a subject that has an EDSS score of greater than 7.0 prior to treatment. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to reduce the frequency of MOGAD relapse to a subject has a negative serum aquaporin-4 antibody titer based on a cell-based assay. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to reduce the frequency of MOGAD relapse to a subject that has a documented history of at least two confirmed MOG antibody disease attacks.

In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to reduce MOG antibody titers. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to reduce or eliminate MOG antibody titers to a subject that has one or more symptoms of MOGAD prior to treatment. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to reduce or eliminate MOG antibody titers and the treatment reduces or eliminates one or more symptoms. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to reduce or eliminate MOG antibody titers to a subject that suffers from loss of color vision, loss or blurring of vision, paralysis/paraparesis of limbs, and loss of bladder/bowel control caused by MOGAD. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to reduce or eliminate MOG antibody titers to a subject that is relapse free about 1 year. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to reduce or eliminate MOG antibody titers to a subject that has at least one confirmed relapse in the previous 12 months or 2 confirmed relapses in the previous 24 months at screening. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to reduce or eliminate MOG antibody titers to a subject that is administered a corticosteroid. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to reduce or eliminate MOG antibody titers to a subject that is administered methylprednisone. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one that is administered to reduce or eliminate MOG antibody titers, wherein after about one year of treatment, the rate of increase of the expanded disability status scale score (EDSS) is reduced. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one that is administered to reduce or eliminate MOG antibody titers, wherein after about one year of treatment, the EDSS score is the same as or reduced from baseline. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one that is administered to reduce or eliminate MOG antibody titers, wherein after about one year of treatment, the rate of increase of the expanded disability status scale score (EDSS) is reduced to a subject that has an EDSS score of greater than 7.0 prior to treatment. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one that is administered to reduce or eliminate MOG antibody titers, wherein after about one year of treatment, the EDSS score is the same as or reduced from baseline to a subject that has an EDSS score of greater than 7.0 prior to treatment. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to reduce or eliminate MOG antibody titers to a subject has a negative serum aquaporin-4 antibody titer based on a cell-based assay. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to reduce or eliminate MOG antibody titers to a subject that has a documented history of at least two confirmed MOG antibody disease attacks.

In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a mammalian subject having MOGAD. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a human subject having MOGAD. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a human subject ranging in age from 12 to 55, having MOGAD. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a human subject ranging in age from 12 to 55, having MOGAD and one or more symptoms of MOGAD prior to treatment. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a human subject ranging in age from 12 to 55, having MOGAD, who suffers from loss of color vision, loss or blurring of vision, paralysis/paraparesis of limbs, and loss of bladder/bowel control caused by MOGAD. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a human subject ranging in age from 12 to 55, having MOGAD, who has an EDSS score of greater than 7.0 prior to treatment. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a human subject ranging in age from 12 to 55, having MOGAD, who has a negative serum aquaporin-4 antibody titer based on a cell-based assay. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a human subject ranging in age from 12 to 55, having MOGAD, who has a documented history of at least two confirmed MOG antibody disease attacks.

In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a subject having MOGAD that is relapse free about 1 year. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a human subject having MOGAD ranging in age from 12 to 55 that is relapse free about 1 year. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a human subject having MOGAD ranging in age from 12 to 55 that is relapse free about 1 year and has one or more symptoms of MOGAD prior to treatment. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a human subject having MOGAD ranging in age from 12 to 55 that is relapse free about 1 year and suffers from loss of color vision, loss or blurring of vision, paralysis/paraparesis of limbs, and loss of bladder/bowel control caused by MOGAD. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a human subject having MOGAD ranging in age from 12 to 55 that is relapse free about 1 year and has an EDSS score of greater than 7.0 prior to treatment. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a human subject having MOGAD ranging in age from 12 to 55 that is relapse free about 1 year and has a negative serum aquaporin-4 antibody titer based on a cell-based assay. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a human subject having MOGAD ranging in age from 12 to 55 that is relapse free about 1 year and has a documented history of at least two confirmed MOG antibody disease attacks.

In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a subject having MOGAD that has at least one confirmed relapse in the previous 12 months or 2 confirmed relapses in the previous 24 months at screening. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a human subject having MOGAD ranging in age from 12 to 55 that has at least one confirmed relapse in the previous 12 months or 2 confirmed relapses in the previous 24 months at screening. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a human subject having MOGAD ranging in age from 12 to 55 that has at least one confirmed relapse in the previous 12 months or 2 confirmed relapses in the previous 24 months at screening and has one or more symptoms of MOGAD prior to treatment. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a human subject having MOGAD ranging in age from 12 to 55 that has at least one confirmed relapse in the previous 12 months or 2 confirmed relapses in the previous 24 months at screening and suffers from loss of color vision, loss or blurring of vision, paralysis/paraparesis of limbs, and loss of bladder/bowel control caused by MOGAD. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a human subject having MOGAD ranging in age from 12 to 55 that has at least one confirmed relapse in the previous 12 months or 2 confirmed relapses in the previous 24 months at screening and has an EDSS score of greater than 7.0 prior to treatment. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a human subject having MOGAD ranging in age from 12 to 55 that has at least one confirmed relapse in the previous 12 months or 2 confirmed relapses in the previous 24 months at screening and has a negative serum aquaporin-4 antibody titer based on a cell-based assay. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered to a human subject having MOGAD ranging in age from 12 to 55 that has at least one confirmed relapse in the previous 12 months or 2 confirmed relapses in the previous 24 months at screening and has a documented history of at least two confirmed MOG antibody disease attacks.

In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered as monotherapy. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered as monotherapy in 60 mg doses. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered as monotherapy in 60 mg doses once daily. In some embodiments, the BTK inhibitor comprising (R)-1-(1-acryloylpiperidin-3-yl)-4-amino-3-(4-phenoxyphenyl)-1H-imidazo[4,5-c]pyridin-2(3H)-one is administered as monotherapy in 60 mg doses once daily with food.

In some embodiments, a dose of about 5-10 mg, 10-15 mg, 15-20 mg, 20-25 mg, 25-30 mg, 30-35 mg, 35-40 mg, 40-45 mg, 45-50 mg, 50-55 mg, or 55-60 mg is administered. In some embodiments, the dose is 5 mg, 10 mg, 15 mg, 20 mg, 25 mg, 30 mg, 35 mg, 40 mg, 45 mg, 50 mg, 55 mg, or 60 mg. In some embodiments, the dose is 5 mg. In some embodiments, the dose is 15 mg. In some embodiments, the dose is 30 mg. In some embodiments, the dose is 60 mg.

In some embodiments, the dose is administered daily. The daily dose can be delivered as a single dose or split into multiple parts. For example, in some embodiments, the dose is administered once a day (e.g., about every 24 hours). In some embodiments, the dose is administered twice daily by having the dose subdivided in two parts to be administered twice per day (e.g., about every 12 hours). In some embodiments, the dose is subdivided in three parts to be administered three times per day (e.g., about every 8 hours). In some embodiments, the dose is subdivided in four parts to be administered four times per day (e.g., about every 6 hours).

In some embodiments, the dose is administered orally. In some embodiments, the dose is administered in a form of tablets. In some embodiments, the dose is administered in the form of pills, capsules, semisolids, powders, sustained release formulations, solutions, suspensions, elixirs, aerosols, or any other appropriate compositions.

In some embodiments, the subject is administered the BTK inhibitor compound for a period of about 1, 2, 3, 4, 5, 6, 7, 8, 9, 10, 11, or 12 months, or for life. In some embodiments, the subject is administered the BTK inhibitor compound for a period of about 12 months. In some embodiments, the dose is once daily. In one embodiment, a method of treating myelin oligodendrocyte glycoprotein antibody disease (MOGAD) is provided, the method comprising administering to a subject in need thereof 60 mg BTK inhibitor comprising (R)-1-(1-acryloylpiperidin-3-yl)-4-amino-3-(4-phenoxyphenyl)-1H-imidazo[4,5-c]pyridin-2(3H)-one.

In some embodiments, a method of treating myelin oligodendrocyte glycoprotein antibody disease (MOGAD) is provided, comprising administering to a subject in need thereof a BTK inhibitor comprising (R)-1-(1-acryloylpiperidin-3-yl)-4-amino-3-(4-phenoxyphenyl)-1H-imidazo[4,5-c]pyridin-2(3H)-one, wherein the BTK inhibitor compound is administered for a period of at least about 12 months. In some embodiments, a method of treating myelin oligodendrocyte glycoprotein antibody disease (MOGAD) is provided, comprising administering to a subject in need thereof a BTK inhibitor comprising (R)-1-(1-acryloylpiperidin-3-yl)-4-amino-3-(4-phenoxyphenyl)-1H-imidazo[4,5-c]pyridin-2(3H)-one, wherein the BTK inhibitor compound is administered once daily for at least about 12 months. In some embodiments, a method of myelin oligodendrocyte glycoprotein antibody disease (MOGAD) is provided, comprising administering to a subject in need thereof a dose of about 5 to about 60 mg of a BTK inhibitor comprising (R)-1-(1-acryloylpiperidin-3-yl)-4-amino-3-(4-phenoxyphenyl)-1H-imidazo[4,5-c]pyridin-2(3H)-one daily. In some embodiments, a method of treating myelin oligodendrocyte glycoprotein antibody disease (MOGAD) is provided, comprising administering to a subject in need thereof a dose of about 5 to about 60 mg of a BTK inhibitor comprising (R)-1-(1-acryloylpiperidin-3-yl)-4-amino-3-(4-phenoxyphenyl)-1H-imidazo[4,5-c]pyridin-2(3H)-one, wherein the BTK inhibitor compound is administered daily for a period of at least about 12 months.

In some embodiments, the BTK inhibitor compound is administered as monotherapy. In some embodiments, the method comprises administering the BTK inhibitor compound and at least one additional therapeutic agent. The additional therapeutic agent may be administered concurrently or sequentially with the BTK inhibitor compound.

Determination of the frequency of administration can be made by persons skilled in the art, such as an attending physician based on considerations of the condition being treated, age of the subject being treated, severity of the condition being treated, general state of health of the subject being treated and the like. In some embodiments, BTK inhibitor compounds are administered in a therapeutically effective amount for treatment of RMS. The therapeutically effective amount is typically dependent on the weight of the subject being treated, his or her physical or health condition, the extensiveness of the condition to be treated, or the age of the subject being treated, pharmaceutical formulation methods, or administration methods (e.g., administration time and administration route).

In some embodiments, a method of treating MOGAD is provided, the method comprising administering to a subject in need thereof a dose of about 5-10 mg, 10-15 mg, 15-20 mg, 20-25 mg, 25-30 mg, 30-35 mg, 35-40 mg, 40-45 mg, 45-50 mg, 50-55 mg, or 55-60 mg of a BTK inhibitor comprising (R)-1-(1-acryloylpiperidin-3-yl)-4-amino-3-(4-phenoxyphenyl)-1H-imidazo[4,5-c]pyridin-2(3H)-one, or a pharmaceutically acceptable salt thereof.

### IV. THERAPEUTIC METHODS TO MITIGATE RISK OF HEPATIC INJURY

In some embodiments, a method of treating myelin oligodendrocyte glycoprotein antibody disease (MOGAD) is provided, comprising the steps of performing an iron panel test using a patient's blood or serum, and if the patient has a suitable iron panel, administering a therapeutically acceptable amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient. In some embodiments the iron panel test measures any one or more of levels of iron, ferritin, transferrin saturation, and total iron-binding capacity (TIBC) in a patient's blood or serum. In some embodiments, a suitable iron panel includes one or more of the following: (i) an iron level of 60 to 170 μg/dL, (ii) a ferritin level of ≤500 g/L (iii) a transferrin saturation level≤50% in a male patient or ≤40% in a female patient, and (iv) a TIBC of 240 to 450 μg/dL.

In some embodiments, a method of treating myelin oligodendrocyte glycoprotein antibody disease (MOGAD) is provided, comprising the steps of performing an iron panel test in a patient's blood or serum, detecting levels of the iron panel test that are within normal ranges, and administering a therapeutically acceptable amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient. In some embodiments the iron panel test measures any one or more of levels of iron, ferritin, transferrin saturation, and total iron-binding capacity (TIBC) in a patient's blood or serum. In some embodiments, the normal ranges of the iron panel test include one or more of (i) an iron level of 60 to 170 μg/dL, (ii) a ferritin level of ≤500 g/L (iii) a transferrin saturation level≤50% in a male patient or ≤40% in a female patient, and (iv) a TIBC of 240 to 450 μg/dL.

In some embodiments, the present disclosure provides a BTK inhibitor comprising (R)-1-(1-acryloylpiperidin-3-yl)-4-amino-3-(4-phenoxyphenyl)-1H-imidazo[4,5-c]pyridin-2(3H)-one (Compound) for use in a method of treating myelin oligodendrocyte glycoprotein antibody disease (MOGAD), comprising the steps of performing an iron panel test in a patient's blood or serum, detecting levels of the iron panel test that are within normal ranges, and administering a therapeutically acceptable amount of Compound to the patient. In some embodiments the iron panel test measures any one or more of levels of iron, ferritin, transferrin saturation, and total iron-binding capacity (TIBC) in a patient's blood or serum. In some embodiments, the normal ranges of the iron panel test include one or more of (i) an iron level of 60 to 170 μg/dL, (ii) a ferritin level of ≤500 g/L (iii) a transferrin saturation level≤50% in a male patient or ≤40% in a female patient, and (iv) a TIBC of 240 to 450 μg/dL.

In some embodiments, a method of treating myelin oligodendrocyte glycoprotein antibody disease (MOGAD) is provided, comprising the steps of determining the level of transferrin saturation in a patient's blood or serum, and if the level of transferrin saturation is suitable, administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient. In some embodiments, a suitable transferrin saturation level in the blood or serum of a male patient is a transferrin saturation of ≤50%. In some embodiments, a suitable transferrin saturation level in the blood or serum of a female patient is a transferrin saturation of ≤40%.

In some embodiments, a method of treating myelin oligodendrocyte glycoprotein antibody disease (MOGAD) is provided, comprising the steps of detecting a level of transferrin saturation in a patient's blood or serum that is within normal range, and administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient. In some embodiments, a transferrin saturation level that is within normal range in the blood or serum of a male patient is a transferrin saturation of ≤50%. In some embodiments, a transferrin saturation level that is within normal range in the blood or serum of a female patient is a transferrin saturation of ≤40%.

In some embodiments, the present disclosure provides a BTK inhibitor comprising (R)-1-(1-acryloylpiperidin-3-yl)-4-amino-3-(4-phenoxyphenyl)-1H-imidazo[4,5-c]pyridin-2(3H)-one (Compound) for use in a method of treating myelin oligodendrocyte glycoprotein antibody disease (MOGAD), comprising the steps of detecting a level of transferrin saturation in a patient's blood or serum that is within normal range, and administering a therapeutically effective amount of Compound to the patient. In some embodiments, a transferrin saturation level that is within normal range in the blood or serum of a male patient is a transferrin saturation of ≤50%. In some embodiments, a transferrin saturation level that is within normal range in the blood or serum of a female patient is a transferrin saturation of ≤40%.

In some embodiments, a method of treating myelin oligodendrocyte glycoprotein antibody disease (MOGAD) is provided, comprising the steps of determining the level of ferritin in a patient's blood or serum, and if the level of ferritin is suitable, administering a therapeutically acceptable amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient. In some embodiments, a suitable ferritin level in the blood or serum of a patient is ≤500 g/L.

In some embodiments, a method of treating myelin oligodendrocyte glycoprotein antibody disease (MOGAD) is provided, comprising the steps of detecting a level of ferritin in a patient's blood or serum that is within normal range, and administering a therapeutically acceptable amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient. In some embodiments, a ferritin level that is within normal range in the blood or serum of a patient is ≤500 g/L.

In some embodiments, the present disclosure provides a BTK inhibitor comprising (R)-1-(1-acryloylpiperidin-3-yl)-4-amino-3-(4-phenoxyphenyl)-1H-imidazo[4,5-c]pyridin-2(3H)-one (Compound) for use in a method of treating myelin oligodendrocyte glycoprotein antibody disease (MOGAD), comprising the steps of detecting a level of ferritin in a patient's blood or serum that is within normal range, and administering a therapeutically acceptable amount of Compound to the patient. In some embodiments, a ferritin level that is within normal range in the blood or serum of a patient is ≤500 g/L.

In some embodiments, a method of treating myelin oligodendrocyte glycoprotein antibody disease (MOGAD) is provided, comprising the steps of performing liver function tests in a patient, and if the patient has suitable liver function, administering a therapeutically acceptable amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient. In some embodiments, the liver function tests measure one or more of the levels of aspartate transaminase (AST), alanine transaminase (ALT), albumin, alkaline phosphatase, total and direct bilirubin, and total protein in a patient's blood. In some embodiments a patient having a suitable liver function has one or more of ALT levels of 1.5× upper limit of normal (ULN), AST levels of 1.5×ULN, alkaline phosphatase 2×ULN (unless caused by non-liver related disorder or explained by a stable chronic liver disorder) and total bilirubin≤1.5×ULN (unless due to Gilbert syndrome or non-liver-related disorder).

In some embodiments, a method of treating myelin oligodendrocyte glycoprotein antibody disease (MOGAD) is provided, comprising the steps of performing liver function tests in a patient, detecting suitable liver function, and administering a therapeutically acceptable amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient. In some embodiments, the liver function tests measure one or more of the levels of aspartate transaminase (AST), alanine transaminase (ALT), albumin, alkaline phosphatase, total and direct bilirubin, and total protein in a patient's blood. In some embodiments a patient having a suitable liver function has one or more of ALT levels of 1.5× upper limit of normal (ULN), AST levels of 1.5×ULN, alkaline phosphatase 2×ULN (unless caused by non-liver related disorder or explained by a stable chronic liver disorder) and total bilirubin≤1.5×ULN (unless due to Gilbert syndrome or non-liver-related disorder).

In some embodiments, the present disclosure provides a BTK inhibitor comprising (R)-1-(1-acryloylpiperidin-3-yl)-4-amino-3-(4-phenoxyphenyl)-1H-imidazo[4,5-c]pyridin-2(3H)-one (Compound) for use in a method of treating myelin oligodendrocyte glycoprotein antibody disease (MOGAD), comprising the steps of performing liver function tests in a patient, detecting suitable liver function, and administering a therapeutically acceptable amount of Compound to the patient. In some embodiments, the liver function tests measure one or more of the levels of aspartate transaminase (AST), alanine transaminase (ALT), albumin, alkaline phosphatase, total and direct bilirubin, and total protein in a patient's blood. In some embodiments a patient having a suitable liver function has one or more of ALT levels of ≤1.5× upper limit of normal (ULN), AST levels of ≤1.5×ULN, and alkaline phosphatase≤2×ULN (unless caused by non-liver related disorder or explained by a stable chronic liver disorder) and total bilirubin≤1.5×ULN (unless due to Gilbert syndrome or non-liver-related disorder).

In some embodiments, the liver function tests are performed at least about every 6 months, at least about every 5 months, at least about every 4 months, at least about every 3 months, at least about every 2 months, or at least about monthly. In some embodiments, the liver function tests are performed at least about every 12 weeks, at least about every 11 weeks, at least about every 10 weeks, at least about every 9 weeks, at least about every 8 weeks, at least about every 7 weeks, at least about every 6 weeks, at least about every 5 weeks, at least about every 4 weeks, at least about every 3 weeks, at least about every 2 weeks, or at least about weekly.

In some embodiments, a method of treating myelin oligodendrocyte glycoprotein antibody disease (MOGAD) is provided, comprising the steps of:
a) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one (Compound) to a patient in need thereof,
b) measuring the level of alanine aminotransferase (ALT) in the patient;
c) detecting a level of ALT of >8× upper limit of normal (ULN);
d) ceasing administration of the Compound to the patient; and optionally
e) monitoring the level of ALT in the patient; and
f) resuming administration of a therapeutically effective amount of the Compound to the patient when the patient's level of ALT is determined to be <1.5×ULN.

In some embodiments, the present disclosure provides a BTK inhibitor comprising (R)-1-(1-acryloylpiperidin-3-yl)-4-amino-3-(4-phenoxyphenyl)-1H-imidazo[4,5-c]pyridin-2(3H)-one (Compound) for use in a method of treating myelin oligodendrocyte glycoprotein antibody disease (MOGAD), comprising the steps of:
a) administering a therapeutically effective amount of Compound to a patient in need thereof;
b) measuring the level of alanine aminotransferase (ALT) in the patient;
c) detecting a level of ALT of >8× upper limit of normal (ULN);
d) ceasing administration of the Compound to the patient; and optionally
e) monitoring the level of ALT in the patient; and
f) resuming administration of a therapeutically effective amount of the Compound to the patient when the patient's level of ALT is determined to be <1.5×ULN.

In some embodiments, a method of treating myelin oligodendrocyte glycoprotein antibody disease (MOGAD) is provided, comprising the steps of:
a) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one (Compound) to a patient in need thereof,
b) measuring the level of alanine aminotransferase (ALT) in the patient;
c) detecting a level of ALT of >5× upper limit of normal (ULN) during a period of at least two weeks;
d) ceasing administration of the Compound to the patient; and optionally
e) monitoring the level of ALT in the patient; and
f) resuming administration of a therapeutically effective amount of the Compound to the patient when the patient's level of ALT is determined to be <1.5×ULN.

In some embodiments, the present disclosure provides a BTK inhibitor comprising (R)-1-(1-acryloylpiperidin-3-yl)-4-amino-3-(4-phenoxyphenyl)-1H-imidazo[4,5-c]pyridin-2(3H)-one (Compound) for use in a method of treating myelin oligodendrocyte glycoprotein antibody disease (MOGAD), comprising the steps of:
a) administering a therapeutically effective amount of Compound to a patient in need thereof;
b) measuring the level of alanine aminotransferase (ALT) in the patient;
c) detecting a level of ALT of >5× upper limit of normal (ULN) during a period of at least two weeks;
d) ceasing administration of the Compound to the patient; and optionally
e) monitoring the level of ALT in the patient; and
f) resuming administration of a therapeutically effective amount of the Compound to the patient when the patient's level of ALT is determined to be <1.5×ULN.

In some embodiments, a method of treating myelin oligodendrocyte glycoprotein antibody disease (MOGAD) is provided, comprising the steps of:
a) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one (Compound) to a patient in need thereof,
b) measuring the level of alanine aminotransferase (ALT) in the patient;
c) detecting a level of ALT of >3× upper limit of normal (ULN);
d) measuring one or more of total bilirubin and international normalized ratio (INR) in a patient;
e) detecting one or more of total bilirubin>2×ULN and INR>1.5;
f) ceasing administration of the Compound to the patient; and optionally
g) monitoring the level of ALT in the patient; and
h) resuming administration of a therapeutically effective amount of the Compound to the patient when the patient's level of ALT is determined to be <1.5×ULN.

In some embodiments, the present disclosure provides a BTK inhibitor comprising (R)-1-(1-acryloylpiperidin-3-yl)-4-amino-3-(4-phenoxyphenyl)-1H-imidazo[4,5-c]pyridin-2(3H)-one (Compound) for use in a method of treating myelin oligodendrocyte glycoprotein antibody disease (MOGAD), comprising the steps of:
a) administering a therapeutically effective amount of Compound to a patient in need thereof;
b) measuring the level of alanine aminotransferase (ALT) in the patient;
c) detecting a level of ALT of >3× upper limit of normal (ULN);
d) measuring one or more of total bilirubin and international normalized ratio (INR) in a patient;
e) detecting one or more of total bilirubin>2×ULN and INR>1.5;
f) ceasing administration of the Compound to the patient; and optionally
g) monitoring the level of ALT in the patient; and
h) resuming administration of a therapeutically effective amount of the Compound to the patient when the patient's level of ALT is determined to be <1.5×ULN.

In some embodiments, a method of treating myelin oligodendrocyte glycoprotein antibody disease (MOGAD) is provided, comprising the steps of:
a) administering a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one (Compound) to a patient in need thereof,
b) measuring the level of alanine aminotransferase (ALT) in the patient;
c) detecting a level of ALT of >3× upper limit of normal (ULN);
d) ceasing administration of the Compound to the patient if the patient experiences one or more of fatigue, nausea, vomiting, right upper quadrant pain or tenderness, fever, rash, and eosinophilia>5%; and optionally
e) monitoring the level of ALT in the patient; and
f) resuming administration of a therapeutically effective amount of the Compound to the patient when the patient's level of ALT is determined to be <1.5×ULN.

In some embodiments, the present disclosure provides a BTK inhibitor comprising (R)-1-(1-acryloylpiperidin-3-yl)-4-amino-3-(4-phenoxyphenyl)-1H-imidazo[4,5-c]pyridin-2(3H)-one (Compound) for use in a method of treating myelin oligodendrocyte glycoprotein antibody disease (MOGAD), comprising the steps of:
a) administering a therapeutically effective amount of Compound to a patient in need thereof;
b) measuring the level of alanine aminotransferase (ALT) in the patient;
c) detecting a level of ALT of >3× upper limit of normal (ULN);
d) ceasing administration of the Compound to the patient if the patient experiences one or more of fatigue, nausea, vomiting, right upper quadrant pain or tenderness, fever, rash, and eosinophilia>5%; and optionally
e) monitoring the level of ALT in the patient; and
f) resuming administration of a therapeutically effective amount of the Compound to the patient when the patient's level of ALT is determined to be <1.5×ULN.

In some embodiments, the ALT level in a patient is measured at least about every 6 months, at least about every 5 months, at least about every 4 months, at least about every 3 months, at least about every 2 months, or at least about monthly. In some embodiments, the ALT level in a patient is measured at least about every 12 weeks, at least about every 11 weeks, at least about every 10 weeks, at least about every 9 weeks, at least about every 8 weeks, at least about every 7 weeks, at least about every 6 weeks, at least about every 5 weeks, at least about every 4 weeks, at least about every 3 weeks, at least about every 2 weeks, or at least about weekly.

In some embodiments, after ceasing administration of the compound, the ALT level in a patient is monitored about every 2 to 3 days, about every 3 days, about every 2 days, or about daily.

In some embodiments, a method of treating myelin oligodendrocyte glycoprotein antibody disease (MOGAD) is provided, comprising administering a therapeutically acceptable amount of a BTK inhibitor comprising (R)-1-(1-acryloylpiperidin-3-yl)-4-amino-3-(4-phenoxyphenyl)-1H-imidazo[4,5-c]pyridin-2(3H)-one to a patient, wherein the patient is not receiving potent and moderate inducers of cytochrome P450 3A (CYP3A) or potent inhibitors of CYP2C8 hepatic enzymes. In some embodiments the potent CYP3A inducers are selected from rifampin, carbamazepine, phenobarbital, St John's Wort extract, avasimibe, lumacaftor, rifapentine, rifabutin, and phenytoin. In some embodiments the moderate CYP3A inducers are selected from semagacestat, asunaprevir, beclabuvir, daclatasvir, cenobamate, nafcillin, lesinurad, modafinil, bosentan, telotristat ethyl, thioridazine, elagolix and rifabutin. In some embodiments, potent CYP2C8 inhibitors are selected from Gemfibrozil and Clopidogrel.

In some embodiments, the present disclosure provides a BTK inhibitor comprising (R)-1-(1-acryloylpiperidin-3-yl)-4-amino-3-(4-phenoxyphenyl)-1H-imidazo[4,5-c]pyridin-2(3H)-one (Compound) for use in a method of treating myelin oligodendrocyte glycoprotein antibody disease (MOGAD), comprising administering a therapeutically acceptable amount of Compound to a patient, wherein the patient is not receiving potent and moderate inducers of cytochrome P450 3A (CYP3A) or potent inhibitors of CYP2C8 hepatic enzymes. In some embodiments the potent CYP3A inducers are selected from rifampin, carbamazepine, phenobarbital, St John's Wort extract, avasimibe, lumacaftor, rifapentine, rifabutin, and phenytoin. In some embodiments the moderate CYP3A inducers are selected from semagacestat, asunaprevir, beclabuvir, daclatasvir, cenobamate, nafcillin, lesinurad, modafinil, bosentan, telotristat ethyl, thioridazine, elagolix and rifabutin. In some embodiments, potent CYP2C8 inhibitors are selected from Gemfibrozil and Clopidogrel.

In some embodiments, a method of treating myelin oligodendrocyte glycoprotein antibody disease (MOGAD) is provided, comprising the steps of advising the patient to limit alcohol consumption during treatment, and administering a therapeutically acceptable amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient. In some embodiments, the patient is female and is advised to limit alcohol consumption to 1 drink per day or less. In some embodiments, 1 drink is approximately 14 grams of alcohol (e.g., 350 mL beer, 140 mL wine, or 40 mL spirits). In some embodiments, the patient is male and is advised to limit alcohol consumption to 2 drinks per day or less. In some embodiments, 2 drinks is approximately 28 grams of alcohol.

In some embodiments, the present disclosure provides a BTK inhibitor comprising (R)-1-(1-acryloylpiperidin-3-yl)-4-amino-3-(4-phenoxyphenyl)-1H-imidazo[4,5-c]pyridin-2(3H)-one (Compound) for use in a method of treating myelin oligodendrocyte glycoprotein antibody disease (MOGAD), comprising the steps of advising the patient to limit alcohol consumption during treatment, and administering a therapeutically acceptable amount of a BTK inhibitor comprising (R)-1-(1-acryloylpiperidin-3-yl)-4-amino-3-(4-phenoxyphenyl)-1H-imidazo[4,5-c]pyridin-2(3H)-one to the patient. In some embodiments, the patient is female and is advised to limit alcohol consumption to 1 drink per day or less. In some embodiments, 1 drink is approximately 14 grams of alcohol (e.g., 350 mL beer, 140 mL wine, or 40 mL spirits). In some embodiments, the patient is male and is advised to limit alcohol consumption to 2 drinks per day or less. In some embodiments, 2 drinks is approximately 28 grams of alcohol.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability. Bioavailability of drugs that decompose at stomach pH can be increased by administration of such drugs in a formulation that releases the drug intraduodenally.

The compositions are comprised of in general, the BTK inhibitor compound or a pharmaceutically acceptable salt thereof in combination with a pharmaceutically acceptable excipient such as binders, surfactants, diluents, buffering agents, antiadherents, glidants, hydrophilic or hydrophobic polymers, retardants, stabilizing agents or stabilizers, disintegrants or superdisintegrants, antioxidants, antifoaming agents, fillers, flavors, colors, lubricants, sorbents, preservatives, plasticizers, or sweeteners, or mixtures thereof, which facilitate processing of the BTK inhibitor compound or a pharmaceutically acceptable salt thereof into preparations which can be used pharmaceutically. Any of the well-known techniques and excipients may be used as suitable and as understood in the art, see for example, Remington: The Science and Practice of Pharmacy, Twenty-first Ed., (Pharmaceutical Press, 2005); Liberman, H. A., Lachman, L., and Schwartz, J. B. Eds., Pharmaceutical Dosage Forms, Vol. 1-2 Taylor & Francis 1990; and R. I. Mahato, Ansel's Pharmaceutical Dosage Forms and Drug Delivery Systems, Second Ed. (Taylor & Francis, 2012).

In certain embodiments, the formulations may include one or more pH adjusting agents or buffering agents, for example, acids such as acetic, boric, citric, fumaric, maleic, tartaric, malic, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate, ammonium chloride, and the like. Such buffers used as bases may have other counterions than sodium, for example, potassium, magnesium, calcium, ammonium, or other counterions. Such acids, bases and buffers are included in an amount required to maintain pH of the composition in an acceptable range.

In certain embodiments, the formulations may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate, or bisulfite anions; suitable salts include sodium chloride, potassium chloride, sodium thiosulfate, sodium bisulfite and ammonium sulfate.

In certain embodiments, the formulations may also include one or more antifoaming agents to reduce foaming during processing which can result in coagulation of aqueous dispersions, bubbles in the finished film, or generally impair processing. Exemplary anti-foaming agents include silicon emulsions or sorbitan sesquoleate.

In certain embodiments, the formulations may also include one or more antioxidants, such as non-thiol antioxidants, for example, butylated hydroxytoluene (BHT), sodium ascorbate, ascorbic acid or its derivative, and tocopherol or its derivatives. In certain embodiments, antioxidants enhance chemical stability where required. Other agents such as citric acid or citrate salts or EDTA may also be added to slow oxidation.

In certain embodiments, the formulations may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide, and cetylpyridinium chloride.

In certain embodiments, the formulations may also include one or more binders. Binders impart cohesive qualities and include, e.g., alginic acid and salts thereof, cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinyl-pyrrolidone/vinyl acetate copolymer; crosspovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum mucilage of isapol husks, polyvinylpyrrolidone (e.g., Polyvidone® CL, Kollidon® CL, Polyplasdone® XL-10), larch arabogalactan, Veegum®, polyethylene glycol, polyethylene oxide, waxes, sodium alginate, and the like.

In certain embodiments, the formulations may also include dispersing agents or viscosity modulating agents. Dispersing agents or viscosity modulating agents include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, H-PC-SL, and HPC-L), hydroxypropyl methylcelluloses (e.g., HPMC K100, RPMC K4M, HPMC K15M, and HPMC K100M), carboxymethylcellulose sodium, methylcellulose, hydroxyethylcellulose, hydroxypropyl-cellulose, hydroxypropylmethylcellulose phthalate, hydroxypropylmethylcellulose acetate stearate (HPMCAS), noncrystalline cellulose, polyethylene oxides, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F10®8, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizcers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate. In general, binder levels of about 10 to about 70% are used in powder-filled gelatin capsule formulations. Binder usage level in tablet formulations varies whether direct compression, wet granulation, roller compaction, or usage of other excipients such as fillers which itself can act as moderate binder. Formulators skilled in art can determine the binder level for the formulations, but binder usage level of up to 90% and more typically up to 70% in tablet formulations is common.

In certain embodiments, the formulations may also include one or more diluents which refer to chemical compounds that are used to dilute the compound of interest prior to delivery. Diluents can also be used to stabilize compounds because they can provide a more stable environment Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

In certain embodiments, the formulations may also include one or more disintegrants which includes both the dissolution and dispersion of the dosage form when contacted with gastrointestinal fluid. Disintegration agents or disintegrants facilitate the breakup or disintegration of a substance. Examples of disintegration agents include a starch, e.g., e.g., a natural starch such as corn starch or potato starch, a pregelatinized starch such as National 1551 or sodium starch glycolate such as Promogel® or Explotab®, a cellulose such as a wood product, methylcrystalline cellulose, e.g., e.g., Avicel®, Avicel® PH101, Avicel® PH 102, Avicel® PH105, Elceme® P100, Emcocel®, Vivacel®, and Solka-Floc®, methylcellulose, croscarmellose, or a cross-linked cellulose, such as cross-linked sodium carboxymethyl-cellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In certain embodiments, the formulations may also include erosion facilitators. Erosion facilitators include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

In certain embodiments, the formulations may also include one or more filling agents which include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

In certain embodiments, the formulations may also include one or more flavoring agents or sweeteners, e.g., acacia syrup, acesulfame K, alitame, anise, apple, aspartame, banana, Bavarian cream berry, black currant, butterscotch, calcium citrate, camphor, caramel, cherry, cherry cream chocolate, cinnamon, bubble gum, citrus, citrus punch, citrus cream, cotton candy, cocoa, cola, cool cherry, cool citrus, cyclamate, cyclamate, dextrose, eucalyptus, eugenol, fructose, fruit punch, ginger, glycyrrhizinate, glycyrrhiza (licorice) syrup, grape, grapefruit, honey, isomalt, lemon, lime, lemon cream, monoammonium glyrrhizinate, maltol, mannitol, maple, marshmallow, menthol, mint cream, mixed berry, neohesperidine DC, neotame, orange, pear, peach, peppermint, peppermint cream, Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, xylitol, sucralose, sorbitol, Swiss cream, tagatose, tangerine, thaumatin, tutti frutti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

In certain embodiments, the formulations may also include one or more lubricants and glidants which are compounds that prevent, reduce or inhibit adhesion or friction of materials. Exemplary lubricants include, e.g., stearic acid, calcium hydroxide, talc, sodium stearyl lumerate, a hydrocarbon such as mineral oil, or hydrogenated vegetable oil such as hydrogenated soybean oil, higher fatty acids and their alkali-metal and alkaline earth metal salts, such as aluminum, calcium, magnesium, zinc, stearic acid, sodium stearates, glycerol, talc, waxes, boric acid, sodium benzoate, sodium acetate, sodium chloride, leucine, a polyethylene glycol (e.g., PEG4000) or a methoxypolyethylene glycol such as Carbowax®, sodium oleate, sodium benzoate, glyceryl behenate, polyethylene glycol, magnesium or sodium lauryl sulfate, colloidal silica such as Syloid®, Cab-O-Sil®, a starch such as corn starch, silicone oil, a surfactant, and the like.

In certain embodiments, the formulations may also include one or more plasticizers which are compounds used to soften the enteric or delayed release coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl citrate, dibutyl sebacate, triethyl cellulose and triacetin. In some embodiments, plasticizers can also function as dispersing agents or wetting agents.

In certain embodiments, the formulations may also include one or more solubilizers which include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins for example Captisol®, ethanol, n-butanol, isopropyl alcohol, cholesterol, bile salts, polyethylene glycol 200-600, glycofurol, transcutol, propylene glycol, and dimethyl isosorbide and the like. In one embodiment, the solubilizer is vitamin E TPGS or Captisol® or ß-hydroxypropylcyclodextrin.

In certain embodiments, the formulations may also include one or more suspending agents which include compounds such as polyvinylpyrrolidone, e.g., polyvinylpyrrolidone K112, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monoleate, povidone and the like.

In certain embodiments, the formulations may also include one or more surfactants which include compounds such as sodium lauryl sulfate, sodium docusate, Tween 20, 60 or 80, triacetin, vitamin E TPGS, sorbitan monooleate, polyoxyethylene sorbitan monooleate, polyoxyethylene sorbitan monolaurate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

In certain embodiments, the formulations may also include one or more viscosity enhancing agents which include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol alginates, acacia, chitosans and combinations thereof.

In certain embodiments, the formulations may also include one or more wetting agents which include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

Pharmaceutical preparations disclosed herein can be obtained by mixing one or more solid excipient such as carrier, binder, filling agent, suspending agent, flavoring agent, sweetening agent, disintegrating agent, dispersing agent, surfactant, lubricant, colorant diluent, solubilizer, moistening agent, plasticizer, stabilizer, penetration enhancer, wetting agent, anti-foaming agent, antioxidant, preservative, or one or more combination thereof with one or more of the compounds described herein, optionally grinding the resulting mixture, and processing the mixture of granules, after adding suitable excipients, if desired, to obtain tablets.

Pharmaceutical preparations disclosed herein also include capsules made of gelatin, as well as soft, sealed capsules made of gelatin and a plasticizer, such as glycerol or sorbitol. Capsules may also be made of polymers such as hypromellose. The capsules can contain the active ingredients in admixture with filler such as lactose, binders such as starches, or lubricants such as talc or magnesium stearate and, optionally, stabilizers. In soft capsules, the active compounds may be dissolved or suspended in suitable liquids, such as fatty oils, liquid paraffin, lipids, solubilizers, or liquid polyethylene glycols. In addition, stabilizers may be added. All formulations for oral administration should be in dosages suitable for such administration.

These formulations can be manufactured by conventional pharmacological techniques. Conventional pharmacological techniques include, e.g., one or a combination of methods: (1) dry mixing, (2) direct compression, (3) milling, (4) dry or non-aqueous granulation, (5) wet granulation, (6) fusion, or (7) extrusion. See, e.g., Lachman et al., The Theory and Practice of Industrial Pharmacy, 3rd ed. (1986). Other methods include, e.g., spray drying, pan coating, melt granulation, granulation, fluidized bed spray drying or coating (e.g., wurster coating), tangential coating, top spraying, tableting, extruding, extrusion/spheronization, and the like.

It should be appreciated that there is considerable overlap between excipients used in the solid dosage forms described herein. Thus, the above-listed additives should be taken as merely exemplary, and not limiting, of the types of excipients that can be included in solid dosage forms described herein. The type and amounts of such excipient can be readily determined by one skilled in the art, according to the particular properties desired.

In some embodiments, the solid dosage forms described herein are enteric coated oral dosage forms, i.e., as an oral dosage form of a pharmaceutical composition as described herein which utilizes an enteric coating to affect the release of the compound in the intestine of the gastrointestinal tract. An “enterically coated” drug or tablet refers to a drug or tablet that is coated with a substance that remains intact in the stomach but dissolves and releases the drug once the intestine (in one embodiment small intestine) is reached. As used herein “enteric coating”, is a material, such as a polymer material or materials which encase the therapeutically active agent core either as a dosage form or as particles. Typically, a substantial amount or all of the enteric coating material is dissolved before the therapeutically active agent is released from the dosage form, so as to achieve delayed dissolution of the therapeutically active agent core or particles in the small or large intestine. Enteric coatings are discussed, for example, Loyd, V. Allen, Remington: The Science and Practice of Pharmacy, Twenty-first Ed., (Pharmaceutical Press, 2005; and P. J. Tarcha, Polymers for Controlled Drug Delivery, Chapter 3, CRC Press, 1991. Methods for applying enteric coatings to pharmaceutical compositions are well known in the art, and include for example, U.S. Patent Publication No. 2006/0045822.

The enteric coated dosage form may be a compressed or molded or extruded tablet (coated or uncoated) containing granules, powder, pellets, beads, or particles of the BTK inhibitor compound or a pharmaceutically acceptable salt thereof or other excipients, which are themselves coated or uncoated provided at least the tablet or the BTK inhibitor compound is coated. The enteric coated oral dosage form may also be a capsule (coated or uncoated) containing pellets, beads, or granules of the BTK inhibitor compound or a pharmaceutically acceptable salt thereof or other excipients, which are themselves coated or uncoated provided at least one of them is coated. Some examples of coatings that were originally used as enteric coatings are beeswax and glyceryl monostearate; beeswax, shellac, and cellulose; and cetyl alcohol, mastic, and shellac as well as shellac and stearic acid (U.S. Pat. No. 2,809,918); polyvinylacetate and ethyl cellulose (U.S. Pat. No. 3,835,221). More recently, the coatings used are neutral copolymers of polymethacrylic acid esters (Eudragit L30D). (F. W. Goodhart et al, Pharm. Tech., p. 64-71, April 1984); copolymers of methacrylic acid and methacrylic acid methyl ester (Eudragit S), or a neutral copolymer of polymethacrylic acid esters containing metallic stearates (Mehta et al U.S. Pat. Nos. 4,728,512 and 4,794,001), cellulose acetate succinate, and hypromellose phthalate.

Any anionic polymer exhibiting a pH-dependent solubility profile can be used as an enteric coating in the methods and compositions described herein to achieve delivery to the intestine. In one embodiment, delivery can be to the small intestine. In another embodiment, delivery can be to the duodenum. In some embodiments the polymers described herein are anionic carboxylic polymers. In other embodiments, the polymers, and compatible mixtures thereof, and some of their properties, include, but are not limited to the following.

Shellac: Also called purified lac, it is a refined product obtained from the resinous secretion of an insect. This coating dissolves in media of pH>7.

Acrylic polymers: The performance of acrylic polymers (primarily their solubility in biological fluids) can vary based on the degree and type of substitution. Examples of suitable acrylic polymers include methacrylic acid copolymers and ammonium methacrylate copolymers. The Eudragit series L, S, and RS (manufactured Rohm Pharma and known as Evonik®) are available as solubilized in organic solvent, aqueous dispersion, or dry powders. The Eudragit series RL, NE, and RS are insoluble in the gastrointestinal tract but are permeable and are used primarily for colonic targeting. The Eudragit series L, L-30D and S are insoluble in stomach and dissolve in the intestine and may be selected and formulated to dissolve at a value of pH greater than 5.5 or as low as greater than 5 or as high as greater than 7.

Cellulose Derivatives: Examples of suitable cellulose derivatives are ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP pseudolatex with particles<1 μm. Other components in Aquateric can include pluronics, Tweens, and acetylated monoglycerides. Other suitable cellulose derivatives include cellulose acetate tritnellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (HPMCAS e.g., AQOAT (Shin Etsu)). The performance can vary based on the degree and type of substitution. For example, HPMCP such as, HP-50, HP-55, HP-55S, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (MF), which dissolves at pH 5.5, and AS-HG (HF), which dissolves at higher pH. These polymers are offered as granules, or as fine powders for aqueous dispersions.

Poly Vinyl Acetate Phthalate (PVAP): PVAP dissolves in pH>5, and it is much less permeable to water vapor and gastric fluids. Detailed description of above polymers and their pH-dependent solubility can be found at in the article titled “Enteric coated hard gelatin capsules” by Professor Karl Thoma and Karoline Bechtold at http://pop.www.capsugel.com/media/library/enteric-coated-hard-gelatin-capsules.pdf. In some embodiments, the coating can, and usually does, contain a plasticizer and possibly other coating excipients such as colorants, talc, or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually contain 10-25% by weight of a plasticizer, especially dibutyl phthalate, polyethylene glycol, triethyl citrate and triacetin. Conventional coating techniques such as fluid bed or Wurster coaters, or spray or pan coating are employed to apply coatings. The coating thickness must be sufficient to ensure that the oral dosage form remains intact until the desired site of topical delivery in the intestinal tract is reached.

Colorants, surfactants, anti-adhesion agents, antifoaming agents, lubricants (e.g., carnauba wax or PEG) and other additives may be added to the coatings besides plasticizers to solubilize or disperse the coating material, and to improve coating performance and the coated product.

To accelerate the dissolution of the enteric coat, a half-thickness, double coat of enteric polymer (for instance, Eudragit L30 D-55) may be applied, and the inner enteric coat may have a buffer up to pH 6.0 in the presence of 10% citric acid, followed by a final layer of standard Eudragit L 30 D-55. Applying two layers of enteric coat, each half the thickness of a typical enteric coat, Liu and Basit were able to accelerate enteric coating dissolution compared to a similar coating system applied, unbuffered, as a single layer (Liu, F. and Basit, A. Journal of Controlled Release. 147 (2010) 242-245.)

The intactness of the enteric coating may be measured, for example, by the degradation of the drug within the micropellets. The enteric coated dosage forms or pellets may be tested in dissolution testing first in gastric fluid and separately in intestinal fluid as described in USP to determine its function.

The enteric coated tablets and capsules formulation containing the disclosed compounds can be made by methods well known in the art. For example, tablets containing a compound disclosed herein can be enterically coated with a coating solution containing Eudragit®, diethylphthlate, isopropyl alcohol, talc, and water using a side vented coating pan (Freund Hi-Coater).

Alternatively, a multi-unit dosage form comprising enteric-coated pellets that can be incorporated into a tablet or into a capsule can be prepared as follows.

Core material: The core material for the individually enteric coating layered pellets can be constituted according to different principles. Seeds layered with the active agent (i.e., the BTK inhibitor compound or a pharmaceutically acceptable sale thereof), optionally mixed with alkaline substances or buffer, can be used as the core material for the further processing. The seeds which are to be layered with the active agent can be water insoluble seeds comprising different oxides, celluloses, organic polymers, and other materials, alone or in mixtures or water-soluble seeds comprising different inorganic salts, sugars, non-pareils and other materials, alone or in mixtures. Further, the seeds may comprise the active agent in the form of crystals, agglomerates, compacts etc. The size of the seeds is not essential for the present disclosure but may range in size approximately from 0.1 to 2 mm. The seeds layered with the active agent are produced either by powder or solution/suspension layering using for instance granulation or spray coating layering equipment.

Before the seeds are layered, active agent may be mixed with further components. Such components can be binders, surfactants, fillers, disintegrating agents, alkaline additives or other or pharmaceutically acceptable ingredients alone or in mixtures. The binders are for example polymers such as hydroxypropyl methylcellulose (HPMC), hydroxypropyl-cellulose (HPC), carboxymethylcellulose sodium, polyvinyl pyrrolidone (PVP), or sugars, starches, or other pharmaceutically acceptable substances with cohesive properties. Suitable surfactants are found in the groups of pharmaceutically acceptable non-ionic or ionic surfactants such as for instance sodium lauryl sulfate.

Alternatively, the active agent optionally mixed with suitable constituents can be formulated into a core material. Said core material may be produced by extrusion/spheronization, balling or compression utilizing conventional process equipment. The size of the formulated core material is approximately from 0.1 to 4 mm, and for example, from 0.1 to 2 mm. The manufactured core material can further be layered with additional ingredients comprising the active agent or be used for further processing.

The active agent is mixed with pharmaceutical constituents to obtain preferred handling and processing properties and a suitable concentration of the active agent in the final preparation. Pharmaceutical constituents such as fillers, binders, lubricants, disintegrating agents, surfactants, and other pharmaceutically acceptable additives may be used.

Alternatively, the aforementioned core material can be prepared by using spray drying or spray congealing technique.

Enteric Coating Layer(s): Before applying the enteric coating layer(s) onto the core material in the form of individual pellets, the pellets may optionally be covered with one or more separating layer(s) comprising pharmaceutical excipients optionally including alkaline compounds such as pH-buffering compounds. This/these separating layer(s), separate(s) the core material from the outer layers being enteric coating layer(s). This/these separating layer(s) protecting the core material of active agent should be water soluble or rapidly disintegrating in water.

A separating layer(s) can be optionally applied to the core material by coating or layering procedures in suitable equipment such as coating pan, coating granulator or in a fluidized bed apparatus using water or organic solvents for the coating process. As an alternative the separating layer(s) can be applied to the core material by using powder coating technique. The materials for the separating layers are pharmaceutically acceptable compounds such as, for instance, sugar, polyethylene glycol, polyvinylpyrrolidone, polyvinyl alcohol, polyvinyl acetate, hydroxypropyl cellulose, methylcellulose, ethylcellulose, hydroxypropyl methyl cellulose, carboxymethylcellulose sodium, water soluble salts of enteric coating polymers and others, used alone or in mixtures. Additives such as plasticizers, colorants, pigments, fillers anti-tacking and anti-static agents, such as for instance magnesium stearate, titanium dioxide, talc and other additives may also be included into the separating layer(s).

When the optional separating layer is applied to the core material it may constitute a variable thickness. The maximum thickness of the separating layer(s) is normally only limited by processing conditions. The separating layer may serve as a diffusion barrier and may act as a pH-buffering zone. The optionally applied separating layer(s) is not essential for the embodiments of the present disclosure. However, the separating layer(s) may improve the chemical stability of the active substance or the physical properties of the novel multiple unit tableted dosage form.

Alternatively, the separating layer may be formed in situ by a reaction between an enteric coating polymer layer applied on the core material and an alkaline reacting compound in the core material. Thus, the separating layer formed comprises a water-soluble salt formed between the enteric coating layer polymer(s) and an alkaline reacting compound which is in the position to form a salt.

One or more enteric coating layers are applied onto the core material or onto the core material covered with separating layer(s) by using a suitable coating technique. The enteric coating layer material may be dispersed or dissolved in either water or in suitable organic solvents. As enteric coating layer polymers, one or more, separately or in combination, of the following can be used, e.g., solutions or dispersions of methacrylic acid copolymers, cellulose acetate phthalate, hydroxypropyl methylcellulose phthalate, hydroxypropyl methylcellulose acetate succinate, polyvinyl acetate phthalate, cellulose acetate trimellitate, carboxymethylethylcellulose, shellac or other suitable enteric coating polymer(s).

The enteric coating layers contain pharmaceutically acceptable plasticizers to obtain the desired mechanical properties, such as flexibility and hardness of the enteric coating layers. Such plasticizers are for instance, but not restricted to triacetin, citric acid esters, phthalic acid esters, dibutyl sebacate, cetyl alcohol, polyethylene glycols, polysorbates or other plasticizers.

The amount of plasticizer is optimized for each enteric coating layer formula, in relation to the selected enteric coating layer polymer(s), selected plasticizer(s) and the applied amount of said polymer(s), in such a way that the mechanical properties, i.e., flexibility and hardness of the enteric coating layer(s), for instance exemplified as Vickers hardness, are adjusted so that if a tablet is desired the acid resistance of the pellets covered with enteric coating layer(s) does not decrease significantly during compression of pellets into tablets. The amount of plasticizer is usually above 5% by weight of the enteric coating layer polymer(s), such as 15-50% and further such as 20-50%. Additives such as dispersants, colorants, pigments polymers e.g., poly(ethylacrylate, methylmethacrylate), anti-tacking and anti-foaming agents may also be included into the enteric coating layer(s). Other compounds may be added to increase film thickness and to decrease diffusion of acidic gastric juices into the acid susceptible material. The maximum thickness of the applied enteric coating is normally only limited by processing conditions and the desired dissolution profile.

Over-Coating Layer: Pellets covered with enteric coating layer(s) may optionally further be covered with one or more over-coating layer(s). The over-coating layer(s) should be water soluble or rapidly disintegrating in water. The over-coating layer(s) can be applied to the enteric coating layered pellets by coating or layering procedures in suitable equipment such as coating pan, coating granulator or in a fluidized bed apparatus using water or organic solvents for the coating or layering process. The materials for over-coating layers are chosen among pharmaceutically acceptable compounds such as, for instance sugar, polyethylene glycol, polyvinylpyrrolidone, polyvinyl alcohol, polyvinyl acetate, hydroxypropyl cellulose, methylcellulose, ethylcellulose, hydroxypropyl methyl cellulose, carboxymethylcellulose sodium and others, used alone or in mixtures. Additives such as plasticizers, colorants, pigments, fillers, anti-tacking, and anti-static agents, such for instance magnesium stearate, titanium dioxide, talc and other additives may also be included into the over-coating layer(s). The over-coating layer may further prevent potential agglomeration of enteric coating layered pellets, further it may protect the enteric coating layer towards cracking during the compaction process and enhance the tableting process. The maximum thickness of the applied over-coating layer(s) is normally limited by processing conditions and the desired dissolution profile. The over-coating layer may also be used as a tablet film coating layer.

Enteric coating of soft gelatin capsules may contain an emulsion, oil, microemulsion, self-emulsifying system, lipid, triglycerides, polyethylene glycol, surfactants, other solubilizers and the like, and combinations thereof, to solubilize the active agent. The flexibility of the soft gelatin capsule is maintained by residual water and plasticizer. Moreover, for gelatin capsules the gelatin may be dissolved in water so that spraying must be accomplished at a rate with relatively low relative humidity such as can be accomplished in a fluid bed or Wurster. In addition, drying should be accomplished without removing the residual water or plasticizer causing cracking of the capsule shell. Commercially available blends optimized for enteric coating of soft gelatin capsules such as Instamodel EPD (Enteric Polymeric Dispersion), available from Ideal Cures, Pvt. Ltd. (Mumbai, India). On a laboratory scale enteric coated capsules may be prepared by: a) rotating capsules in a flask or dipping capsules in a solution of the gently heated enteric coating material with plasticizer at the lowest possible temperature or b) in a lab scale sprayer/fluid bed and then drying.

For aqueous active agents, it can be especially desirable to incorporate the drug in the water phase of an emulsion. Such “water-in-oil” emulsion provides a suitable biophysical environment for the drug and can provide an oil-water interface that can protect the drug from adverse effects of pH or enzymes that can degrade the drug. Additionally, such water-in-oil formulations can provide a lipid layer, which can interact favorably with lipids in cells of the body, and can increase the partition of the formulation onto the membranes of cells. Such partition can increase the absorption of drugs in such formulations into the circulation and therefore can increase the bioavailability of the drug.

In some embodiments the water-in-oil emulsion contains an oily phase composed of medium or long chain carboxylic acids or esters or alcohols thereof, a surfactant or a surface-active agent, and an aqueous phase containing primarily water and the active agent.

Medium and long chain carboxylic acids are those ranging from C8 to C22 with up to three unsaturated bonds (also branching). Examples of saturated straight chain acids are n-dodecanoic acid, n-tetradecanoic acid, n-hexadecanoic acid, caproic acid, caprylic acid, capric acid, lauric acid, myristic acid, palmitic acid, stearic acid, arachidic acid, behenic acid, montanic acid and melissic acid. Also useful are unsaturated monoolefinic straight chain monocarboxylic acids. Examples of these are oleic acid, gadoleic acid and erucic acid. Also useful are unsaturated (polyolefinic) straight chain monocarboxylic acids. Examples of these are linoleic acid, ricinoleic acid, linolenic acid, arachidonic acid and behenolic acid. Useful branched acids include, for example, diacetyl tartaric acid. Unsaturated olefinic chains may also be hydroxylated or ethoxylated to prevent oxidation or to alter the surface properties.

Examples of long chain carboxylic acid esters include, but are not limited to, those from the group of: glyceryl monostearates; glyceryl monopalmitates; mixtures of glyceryl monostearate and glyceryl monopalmitate; glyceryl monolinoleate; glyceryl monooleate; mixtures of glyceryl monopalmitate, glyceryl monostearate, glyceryl monooleate and glyceryl monolinoleate; glyceryl monolinolenate; glyceryl monogadoleate; mixtures of glyceryl monopalmitate, glyceryl monostearate, glyceryl monooleate, glyceryl monolinoleate, glyceryl monolinolenate and glyceryl monogadoleate; acetylated glycerides such as distilled acetylated monoglycerides; mixtures of propylene glycol monoesters, distilled monoglycerides, sodium steroyl lactylate and silicon dioxide; d-alpha tocopherol polyethylene glycol 1000 succinate; mixtures of mono- and di-glyceride esters such as Atmul; calcium stearoyl lactylate; ethoxylated mono- and di-glycerides; lactated mono- and di-glycerides; lactylate carboxylic acid ester of glycerol and propylene glycol; lactylic esters of long chain carboxylic acids; polyglycerol esters of long chain carboxylic acids, propylene glycol mono- and di-esters of long chain carboxylic acids; sodium stearoyl lactylate; sorbitan monostearate; sorbitan monooleate; other sorbitan esters of long chain carboxylic acids; succinylated monoglycerides; stearyl monoglyceryl citrate; stearyl heptanoate; cetyl esters of waxes; stearyl octanoate; C8-C30 cholesterol/lavosterol esters; and sucrose long chain carboxylic acid esters. Examples of the self-emulsifying long chain carboxylic acid esters include those from the groups of stearates, palmitates, ricinoleates, oleates, behenates, ricinolenates, myristates, laurates, caprylates, and caproates. In some embodiments the oily phase may comprise a combination of 2 or more of the long chain carboxylic acids or esters or alcohols thereof. In some embodiments medium chain surfactants may be used and the oil phase may comprise a mixture of caprylic/capric triglyceride and C8/C10 mono-/di-glycerides of caprylic acid, glyceryl caprylate or propylene glycol monocaprylate or their mixtures.

The alcohols that can be used are exemplified by the hydroxyl forms of the carboxylic acids exemplified above and also stearyl alcohol.

Surface active agents or surfactants are long chain molecules that can accumulate at hydrophilic/hydrophobic (water/oil) interfaces and lower the surface tension at the interface. As a result, they can stabilize an emulsion. In some embodiments, the surfactant may comprise: Tween® (polyoxyethylene sorbate) family of surfactants, Span® (sorbitan long chain carboxylic acid esters) family of surfactants, Pluronic® (ethylene or propylene oxide block copolymers) family of surfactants, Labrasol®, Labrafil® and Labrafac® (each polyglycolyzed glycerides) families of surfactants, sorbitan esters of oleate, stearate, laurate or other long chain carboxylic acids, poloxamers (polyethylene-polypropylene glycol block copolymers or Pluronic®), other sorbitan or sucrose long chain carboxylic acid esters, mono and diglycerides, PEG derivatives of caprylic/capric triglycerides and mixtures thereof or mixture of two or more of the above. In some embodiments the surfactant phase may comprise a mixture of Polyoxyethylene (20) sorbitan monooleate (Tween 80®) and sorbitan monooleate (Span 80®).

The aqueous phase may optionally comprise the active agent suspended in water and a buffer.

In some embodiments, such emulsions are coarse emulsions, microemulsions and liquid crystal emulsions. In other embodiments such emulsion may optionally comprise a permeation enhancer. In other embodiments, spray-dried dispersions or microparticles or nanoparticles containing encapsulated microemulsion, coarse emulsion or liquid crystal can be used.

In some embodiments, the solid dosage forms described herein are non-enteric time-delayed release dosage forms. The term “non-enteric time-delayed release” as used herein refers to the delivery so that the release of the drug can be accomplished at some generally predictable location in the intestinal tract more distal to that which would have been accomplished if there had been no delayed release alterations. In some embodiments the method for delay of release is a coating that becomes permeable, dissolves, ruptures, or is no longer intact after a designed duration. The coating in the time-delayed release dosage forms can have a fixed time to erode after which the drug is released (suitable coating include polymeric coating such as HPMC, PEO, and the like) or has a core comprised of a superdisintegrant(s) or osmotic agent(s) or water attractant such as a salt, hydrophilic polymer, typically polyethylene oxide or an alkylcellulose, salts such as sodium chloride, magnesium chloride, sodium acetate, sodium citrate, sugar, such as glucose, lactose, or sucrose, or the like, which draw water through a semi-permeable membrane or a gas generating agent such as citric acid and sodium bicarbonate with or without an acid such as citric acid or any of the aforementioned acids incorporated in dosage forms. The semi-permeable membrane, while mostly not permeable to the drug nor the osmotic agent, is permeable to water that permeates at a near constant rate to enter the dosage form to increase the pressure and ruptures after the swelling pressure exceeds a certain threshold over a desired delay time. The permeability through this membrane of the drug should be less than 1/10 than water and in one embodiment less than 1/100 the water permeability. Alternatively, a membrane could become porous by leaching an aqueous extractable over a desired delay time.

Osmotic dosage forms have been described in Theeuwes U.S. Pat. No. 3,760,984, and an osmotic bursting dosage form is described in Baker U.S. Pat. No. 3,952,741. This osmotic bursting dosage form can provide a single pulse of release or multiple pulses if different devices with different timings are employed. The timing of the osmotic burst may be controlled by the choice of polymer and the thickness or the area of the semipermeable membrane surrounding the core that contains both the drug and the osmotic agent or attractant. As the pressure in the dosage form increase with additional permeated water, the membrane elongates until its breaking point, and then the drug is released. Alternatively, specific areas of rupture can be created in the membrane by having a thinner, weaker area in the membrane or by adding a weaker material to an area of the coating membrane. Some preferred polymers with high water permeabilities that may be used as semipermeable membranes are cellulose acetate, cellulose acetate butyrate, cellulose nitrate, crosslinked polyvinyl, alcohol, polyurethanes, nylon 6, nylon 6.6, and aromatic nylon. Cellulose acetate is an especially preferred polymer.

In another embodiment, the time-delayed coating that begins its delay to releasing drug after the enteric coating is at least partially dissolved is comprised of hydrophilic, erodible polymers that upon contact with water begin to gradually erode over time. Examples of such polymers include cellulose polymers and their derivatives including, but not limited to, hydroxyalkyl celluloses, hydroxymethyl cellulose, hydroxyethyl cellulose, hydroxypropyl cellulose, hydroxypropylmethyl cellulose, carboxymethylcellulose, microcrystalline cellulose; polysaccharides and their derivatives; polyalkylene oxides, such as polyethylene oxide or polyethylene glycols, particularly high molecular weight polyethylene glycols; chitosan; poly(vinyl alcohol); xanthan gum; maleic anhydride copolymers; poly(vinyl pyrrolidone); starch and starch-based polymers; maltodextrins; poly (2-ethyl-2-oxazoline); poly(ethyleneimine); polyurethane; hydrogels; crosslinked polyacrylic acids; and combinations or blends of any of the foregoing.

Some preferred erodible hydrophilic polymers suitable for forming the erodible coating are poly(ethylene oxide), hydroxypropyl methyl cellulose, and combinations of poly(ethylene oxide) and hydroxypropyl methyl cellulose. Poly(ethylene oxide) is used herein to refer to a linear polymer of unsubstituted ethylene oxide. The molecular weight of the poly(ethylene oxide) polymers can range from about 105 Daltons to about 107 Daltons. A preferred molecular weight range of poly(ethylene oxide) polymers is from about 2×105 to 2×106 Daltons and is commercially available from The Dow Chemical Company (Midland, Mich.) referred to as SENTRYR POLYOX™ water-soluble resins, NF (National Formulary) grade. When higher molecular weights of polyethylene oxide are used, other hydrophilic agents, such as salts or sugars, like glucose, sucrose, or lactose, that promote erosion or disintegration of this coating, are also included.

The time-delayed dosage form can be a mechanical pill such as an Enterion® capsule or pH sensitive capsule which can release the drug after a pre-programmed time or when it receives a signal which can be transmitted or once it leaves the stomach.

The amount of the compound of the disclosure in a formulation can vary within the full range employed by those skilled in the art. Typically, the formulation will contain, on a weight percent (wt %) basis, from about 0.01-99.99 wt % of the BTK inhibitor compound based on the total formulation, with the balance being one or more suitable pharmaceutical excipients. In one embodiment, the compound is present at a level of about 1-80 wt %.

The foregoing disclosure has been described in some detail by way of illustration and example, for purposes of clarity and understanding. Therefore, it is to be understood that the above description is intended to be illustrative and not restrictive. The scope of the disclosure should, therefore, be determined not with reference to the above description, but should instead be determined with reference to the following appended claims, along with the full scope of equivalents to which such claims are entitled.

### EXAMPLES

The following examples are provided to illustrate certain disclosed embodiments and are not to be construed as limiting the scope of this disclosure in any way. In the Examples discussed below, the BTK inhibitor, as defined above, may be also referred as “the compound” or “the drug” interchangeably.

### Example 1—MOG Antibody Disease (MOGAD) Study of BTK Inhibitor Tolebrutinib

### Example 1.1—Introduction and Study Rationale

Tolebrutinib is a brain penetrant inhibitor of Bruton's tyrosine kinase (BTK). This compound is being developed for the treatment of MOG antibody disease (MOGAD). This study is designed to collect evidence of its efficacy and safety in an active relapsing MOGAG population.

MOGAD is a relapsing, immune mediated disease of the central nervous system that leads to vision loss, weakness, sensory loss, bowel/bladder dysfunction and cognitive dysfunction (Narayan et al. Mult Scler Relat Disord 2018, 25, 66-72). On the spectrum of CNS autoimmune diseases, MOG antibody disease overlaps with multiple sclerosis (MS). Pathologically, anti-MOG appears similar to MS with demyelination and macrophage infiltration with intact axons and limited humoral deposits (Hoftberger et al. Acta Neuropathol 2020, 139, 875-892). Clinically, attacks of MOGAD show a predilection for the optic nerves (66% of attacks) and unlike MS, there is no known progressive course of disease in MOG. That is, if relapses can be prevented, the disease is effectively in remission.

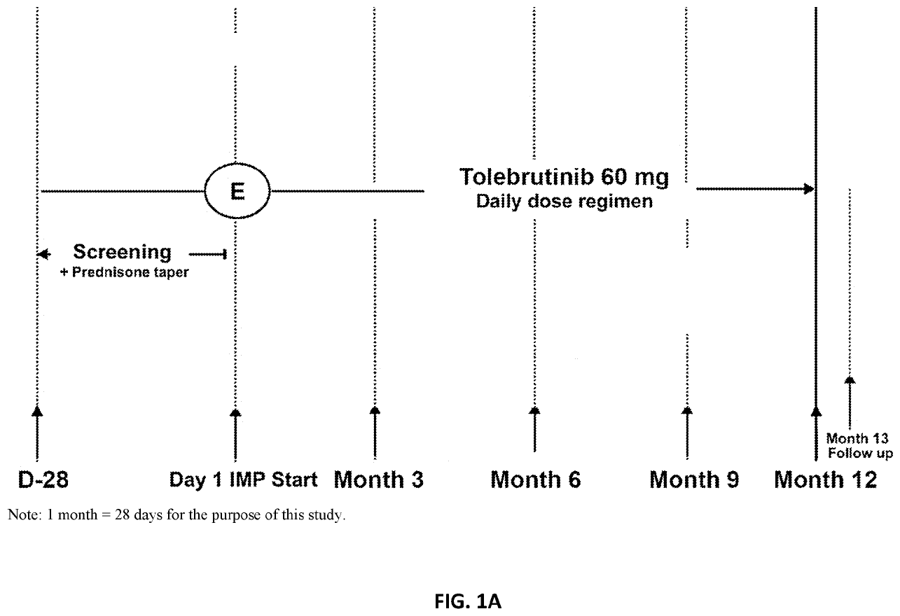

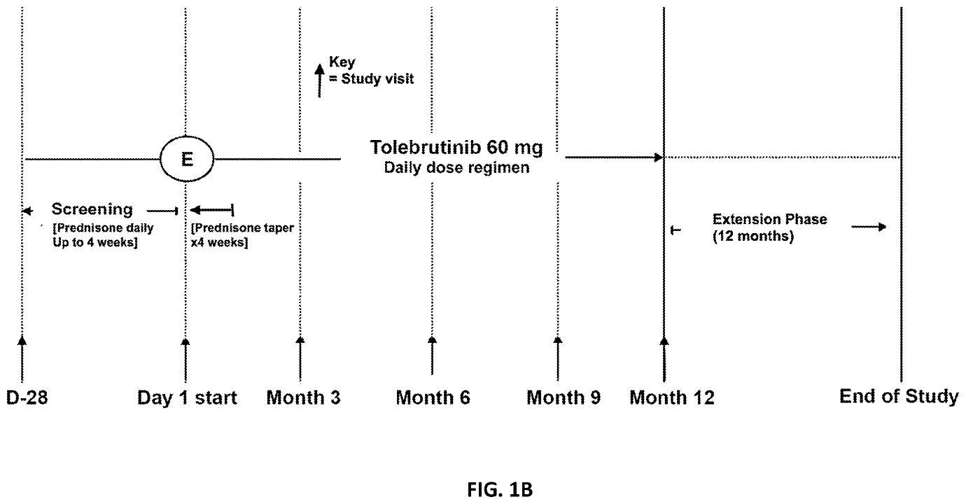

A graphical scheme of the study design is shown in FIG. 1A. Table 1 shown below describes the schedule of activities during the course of study. Table 2 that follows describes the objective and endpoints of the overall study.

TABLE 1
Schedule of Activities (SOA)

| | | | | | | | Relapse | Follow-up | | |
|---|---|---|---|---|---|---|---|---|---|---|
| | Screening | Intervention Period | | | | | Evaluation | Relapse Visit | Follow-up | Premature end |
| | (up to 28 days | (every 90 days) | | | | | event- | 4 weeks after | Visit | of treatment |
| Procedure | before Day 1) | D 1 | M 3 | M 6 | M 9 | M 12 | triggered | relapse onset | M 13 | N/A |
| Virtual Visit | 1 | 2 | 3 | 4 | 5 | 6 | As needed | As needed | (if no | N/A |
| | | | | | | | | | extension) | |
| Informed consent | X | | | | | | | | | |
| Inclusion and | X | X | | | | | | | | |
| exclusion criteria | | | | | | | | | | |
| Demography | X | | | | | | | | | |
| Height and weight | | X | | | | | | | | |
| (informed by | | | | | | | | | | |
| participant from | | | | | | | | | | |
| last medical visit or | | | | | | | | | | |
| measured locally) | | | | | | | | | | |
| EDSS (measured | X | X | X | X | X | X | X | X | X | X |
| remotely) | | | | | | | | | | |
| ECG and vital | X | X | | X | | X | | | X | X |
| signs | | | | | | | | | | |
| (done/measured | | | | | | | | | | |
| locally) | | | | | | | | | | |
| Medical and | X | | | | | | X | X | X | |
| Neurological | | | | | | | | | | |
| history | | | | | | | | | | |
| MOG Ab titer | X | | | | | X | | | | X |
| MRI (brain, optic | | | | | | | X | X | | |
| nerves or spinal | | | | | | | | | | |
| cord) | | | | | | | | | | |
| Screening and D 1 | X | X | | | | | | | | |
| labs* | | | | | | | | | | |
| Safety monitoring | | | | X | | X | X | X | X | X |
| labs** | | | | | | | (if needed) | (if needed) | | |
| Urine pregnancy | | | X | X | X | X | | | X | X |
| test (done at | | | | | | | | | | |
| home)*** | | | | | | | | | | |
| C-SSRS | X | X | | X | | X | | | X | X |
| AE/SAE review | | X | X | X | X | X | | | X | X |
| Biomarkers**** | | X | | | | X | | | | |
| OCT (RNFL and | | X | | | | X | | | | |
| GCIPL thickness), | | | | | | | | | | |
| if available locally | | | | | | | | | | |

Abbreviations: AE: adverse event; AST: aspartate aminotransferase; ALT: alanine aminotransferase; C-SSRS: Columbia Suicide Severity Rating Scale; D: day; ECG: electrocardiogram; EDSS: Expanded Disability Status Scale; GCIPL: ganglion cell-inner plexiform layer; HIV: human immunodeficiency virus; Ig: immunoglobulin; IMP: investigational medicinal product; INR: international normalized ratio M: month (28 days); MRI: magnetic resonance imaging; MOG: myelin-oligodendrocyte glycoprotein; OCT: optical coherence tomography; RNFL: retinal nerve fiber layer; SAE: serious adverse event. The participant should return for a follow-up visit 2 to 4 weeks after premature end-of-treatment.
*Screening and D 1 labs: pregnancy test (serum), complete blood count with differential, coagulation (only at screening), metabolic profile (blood urea nitrogen [BUN], creatinine, glucose [nonfasting], potassium, sodium, chloride, bicarbonate, calcium, liver function tests [AST, ALT, albumin, alkaline phosphatase, total and direct bilirubin, total protein; creatine phosphokinase]), lipase (only at screening). IgG and IgM levels (only on D 1 and M 12), blood testing (eg, QuantiFERON ® TB Gold test) is preferred for tuberculosis (only at screening), hepatitis B/C serology (only at screening), HIV serology (if locally allowed, only at screening), Urinalysis (only at screening, M 3 and M 12); follicle-stimulating horm (FSH) (if needed, only in female participants to confirm postmenopausal state at screening); serum anti-aquaporin 4 antibody (only at screening
**Safety monitoring labs: complete blood count with differential, liver function test and metabolic profile. Urinalysis (M 3 and M 12). Pregnancy tests of ay types can be requested anytime during the course of the study as determined necessary by the PI or local regulation.
***Urine pregnancy test: to be done at home - from M 3 to M 12 (quarterly) and safety F/U visit - Communication by phone to the site of the result of a pregnancy test performed at home is allowed.
****Biomarkers (blood/serum): neurofilament light chain, glial fibrillary acidic protein level, tau, S100B and B/T cell profiling (potentially in a future analysis). Optional for participants who have relapsed during the course of the trial.

Objectives and endpoints for the treatment are shown in Table 2.

TABLE 2
Objectives and endpoints

| Objectives | Endpoints |
|---|---|
| Primary | |
| To assess the efficacy of tolebrutinib | Proportion of relapse-free participants |
| To prevent relapses in participants with | Time to first relapse |
| MOG antibody disease | |
| Secondary | |
| To assess the safety and tolerability of | Adverse events (AEs), serious AEs, AEs |
| tolebrutinib in participants with MOG | leading to permanent study intervention |
| antibody disease | discontinuation, AEs of special interest, |
| | and potentially clinically significant |
| | abnormalities in laboratory tests, safety |
| | scales, ECG, and vital signs during the |
| | study period |
| To determine the effect of tolebrutinib on | Change from baseline in MOG antibody |
| MOG antibody titer | titer level after 1 year of treatment with |
| | tolebrutinib |
| To quantify the impact of tolebrutinib on | Change from baseline in expanded |
| neurological disability accumulation | disability status scale score (EDSS) after |
| | 1 year of treatment with tolebrutinib |
| Tertiary/Exploratory/Other | Changes from baseline in measures of |
| To explore effects of tolebrutinib on | CNS damage including OCT (RNFL and |
| recovery and remyelination | GCIP thickness), MRI (size and number |
| | of T2 lesions in the brain, optic nerve, |
| | and spinal cord), and serum (GFAP, |
| | S100B, tau, neurofilament light) after 1 |
| | year of treatment with tolebrutinib among |
| | patients who do not relapse. |

### Appropriateness of Measurements

MOG antibody disease appears to accumulate disability in a stepwise progression driven by the relapses that occasionally leads to sustained loss of neurological function (Jurynczyk et al. Brain 2017, 140, 3128-3138). Hence, reducing the relapse frequency is a clinically relevant outcome and is an aim of any potential disease-modifying therapy.

Monophasic patients never relapse. These monophasic patients may not be part of the study (as an inclusion criteria requires at least 2 attacks). Roughly half of the patients will experience a second attack and/or additional relapses. See for example, Akaishi et al. J Neurol 2021; Kornberg et al. Neurology, 2021. Reduction of relapse frequency is the goal; the patient's residual disability worsens after an attack. The treatment goal is to administer therapy to prevent and/or reduce the likelihood that an additional attack occurs in a patient. For additional information, see Alessandro Dinoto, et al. “Serum and Cerebrospinal Fluid Biomarkers in Neuromyelitis Optica Spectrum Disorder and Myelin Oligodendrocyte Glycoprotein Associated Disease” Frontiers in Neurology, March 2022, Volume 13, Article 866824.

Persistent MOG antibody titers for more than 6 months are related to a higher risk of a relapsing phenotype that could be present in almost 88% of MOG antibody disease patients (Lopez-Chiriboga et al. JAMA Neurol 2018. 75, 1355-1363). There is also preliminary evidence showing that high MOG antibody titers could be associated with a more aggressive disease (Tea et al. Acta Neuropathol Commun 2019, 7, 145). Therefore, reducing or eliminating MOG antibody titers may be a positive outcome of the treatment with a potential disease-modifying therapy.

The presence of MOG antibody is analyzed by an extracellular live cell-staining immunofluorescence technique using transfected MOG-expressing cells. Antibody titers are assessed by measuring the fluorescence intensity signals via flow cytometry of serial dilutions.

Reduction of MOG antibody titer is being observed. It is still to be established whether this biomarker can be reliably used to monitor disease state. If the antibody titer decreases or is eliminated (conversion from positive to negative)—that would constitute a reduction of MOG titer. In technical terms, if MOG antibody titer level forms a negative slope after 1 year of treatment with tolebrutinib, that would constitute a reduction of MOG antibody titer. Likewise, in technical terms, elimination of MOG antibody titer is when the fluorescence intensity signals from flow cytometry displays same level with the non-transfected negative control cell after 1 year of treatment with tolebrutinib.

Measurement of neurological disability accumulation is an important clinical endpoint in demyelinating diseases of the central nervous system. The EDSS is widely used to measure neurological disability in clinical trials and routine settings. Given the short duration of this pilot study, change from baseline in expanded disability status scale score (EDSS) 1 year after initiation of treatment with tolebrutinib will be measured as a secondary endpoint.

In addition to the above-mentioned assessments, MRI and OCT findings and serum/blood biomarkers measurements will be anaylzed in this study as exploratory supportive efficacy data.

### Example 1.2—Study Design

### Example 1.2A—Overall Design

This is a Phase 2, open-label, single arm, single center trial to assess the efficacy and safety of 1-year tolebrutinib treatment in participants with MOG antibody disease. Participants will be screened to assess their eligibility based on the study inclusion and exclusion criteria. After screening, eligible participants will be enrolled to receive 60 mg of tolebrutinib oral daily dose. The primary endpoint will be the proportion of participants relapse free 1 year after enrollment.

The study will be conducted entirely remotely with no in-person visits to the site. Virtual visits every 3 months after enrollment will assess limited neurological function, MOG antibody titer and safety measures. In addition to routine virtual evaluations, relapse evaluations will be triggered if participants report new neurological symptoms consistent with a MOG antibody disease relapse and include an MRI of the optic nerves or spinal cord or brain depending on the clinical presentation and associated neurologic signs and symptoms. Participants who meet criteria for a relapse will be treated for the relapse by their local doctor (according to the local standard of care) and have a remote follow-up relapse visit scheduled for 4 weeks after the onset of the relapse symptoms; then advised to discontinue the study drug. Participants will be encouraged to remain in the trial even if the study drug is discontinued.

### Example 1.2B—Number of Participants

Approximately 30 participants will be screened to achieve 25 enrolled to receive the study intervention.

### Example 1.2C—Intervention Groups and Duration

All participants will be screened over 4 weeks to review the consent form, wean off background immunotherapy, and confirm eligibility for the trial. Participants will use 20 mg daily prednisone through the 4 week screening period while weaning off background immunotherapy. Prednisone will then be weaned down by 5 mg daily every week at the start of the 1-year treatment period, concurrent with use of 60 mg daily tolebrutinib. No additional prednisone or background immunotherapy is permitted during the 1-year trial period except as treatment for a confirmed relapse.

### Example 1.2D—Rationale

This is a Phase 2 study for which an open-label design has been selected to avoid participants' unnecessary exposure to placebo and to preliminarily explore the efficacy and the safety of tolebrutinib in an underserved population. Male and female participants with a diagnosis of MOGAD ranging in age from 12 to 55 years will be selected for this study. The age range is limited to participants≤55 years to reduce confounding neurological conditions prevalent in older individuals (eg, degenerative pathology of the spine, vascular disorders, other neurodegenerative processes). The dose for participants ranging in age from 12 to 17 years will be informed by the results of a PK extrapolation modeling. The intended duration of the treatment period is 12 months and will be fixed for all individual participants. Participants will be encouraged to remain in the study and comply with all study visits until the M12 visit in the case that they discontinue the study intervention early. With an average relapse rate of 1 attack per year, 12 months is an adequate time period to evaluate the efficacy of tolebrutinib to prevent attacks.

### Example 1.2E—Dose Regimen

The choice of the dose of 60 mg of the BTK inhibitor taken with food is based on the results of a Phase 2b dose-finding trial for tolebrutinib in participants with relapsing multiple sclerosis (DRI15928). See for example, Reich, D. S., et al., Safety and efficacy of tolebrutinib, an oral brain-penetrant BTK inhibitor, in relapsing multiple sclerosis: a phase 2b, randomised, double-blind, placebo-controlled trial. Lancet Neurol, 2021. 20(9): p. 729-738.

Analysis of the PK data and effect of fed status on tolebrutinib exposure showed a positive food effect with an increase in AUC0-24 of approximately 2-fold. Moreover, the correlation between the treatment response and the exposure to tolebrutinib showed that higher exposure was associated with low numbers of new gadolinium-enhancing T1-hyperintense lesions after 12 weeks of treatment. Taken together, these data support the recommendation to take tolebrutinib with food.

There was no correlation between the dose of tolebrutinib administered and the number of TEAEs. The most common events (preferred terms) observed in participants in the tolebrutinib treatment arms were headache, upper respiratory tract infection, and nasopharyngitis. There were low numbers of AESIs and PCSAs observed. Overall, no new risks were identified in this trial.

### Example 1.2F—End of Study Definition

A participant is considered to have completed the study if he/she has completed all phases of the study including the M12. Participants who have a relapse during the course of the trial will be considered as completers if they discontinue the study drug and complete the premature end of treatment visit. This study will end when approximately 25 participants complete the M12 visit. With a planned recruitment period of approximately 12 months and a study duration per participant of approximately 12 months, the estimated duration of the study is approximately 24 months.

### Example 1.3—Study Population

### Example 1.3A—Inclusion Criteria

Participants are eligible to be included in the study only if all of the following criteria apply as shown in Table 3.

TABLE 3
Inclusion Criteria

| Category | Criteria |
|---|---|
| Age | I 01. The participant must be 12 to 55 years of age inclusive, at the |
| | time of signing the informed consent. |
| Type of | I 02. The participant must have a current diagnosis of MOG |
| participant | antibody disease according to MOG encephalomyelitis: |
| and disease | international recommendations on diagnosis and antibody |
| characteristics | testing (Jarius et al. J Neuroinflammation 2018, 15, 134). |
| | I 03. The participant must have an EDSS score <7.0 at screening. |
| | I 04. The participant must have negative serum aquaporin-4 |
| | antibodies (cell-based assay is the only accepted method). |
| | I 05. The participant must have a documented history of at least 2 |
| | confirmed MOG antibody disease attacks. |
| | I 06. The participant must have had at least one confirmed relapse in |
| | the previous 12 months or 2 confirmed relapses in the previous |
| | 24 months at screening. |
| Weight | I 07. Not Applicable. |
| Sex | I 08. Male or Female. |
| | Contraceptive use should be consistent with local regulations |
| | regarding the methods of contraception for those participating |
| | in clinical studies. |
| | a) Male participants |
| | Not applicable. |
| | b) Female participants |
| | A female participant is eligible to participate if she is not |
| | pregnant or breastfeeding, and at least one of the following |
| | conditions applies: |
| | Is not a woman of childbearing potential (WOCBP); or |
| | Is a WOCBP and agrees to use an acceptable contraceptive |
| | method during the intervention period (at a minimum until |
| | after the last dose of study intervention). |
| | A WOCBP must have a negative highly sensitive |
| | pregnancy test (urine or serum as required by local |
| | regulations) (Example 1.8A) at screening before the |
| | first dose of study intervention. If a urine test cannot be |
| | confirmed as negative (eg, an ambiguous result), a serum |
| | pregnancy test is required. In such cases, the participant |
| | must be excluded from participation if the serum |
| | pregnancy result is positive. |
| | Requirements for pregnancy testing during and after study |
| | intervention are located in the schedule of activities (SoA); |
| | Example 1.1). |
| | The Investigator is responsible for review of medical |
| | history, menstrual history, and recent sexual activity to |
| | decrease the risk for inclusion of a woman with an early |
| | undetected pregnancy, if allowed by local regulations. |
| Informed | I 09. Capable of giving signed informed consent which includes |
| Consent | compliance with the requirements and restrictions listed in the |
| | informed consent form (ICF). A specific ICF for legally minor |
| | participants must also be signed by the participant's legally |
| | authorized representative. |

### Example 1.3B—Exclusion Criteria

Participants are excluded from the study if any of the following criteria apply as shown in Table 4.

TABLE 4
Exclusion Criteria

| Category | Criteria | |
|---|---|---|
| Medical | E 01. The participant has a history of infection or may be at risk | |
| conditions | for infection: | |
| | A history of T-lymphocyte or T-lymphocyte-receptor | |
| | vaccination, transplantation (including solid organ, stem | |
| | cell, and bone marrow transplantation) or antirejection | |
| | therapy. | |
| | The participant has received any live (attenuated) vaccine | |
| | (including but not limited to varicella zoster, oral polio, | |
| | and nasal influenza) within 2 months before the first | |
| | treatment visit. | |
| | The participant has a lymphocyte count less than the lower | |
| | limit of normal (LLN) at the Screening Visit. | |
| | A history of diagnosis of progressive multifocal | |
| | leukoencephalopathy (PML) or evidence of findings | |
| | suggestive of PML on the screening MRI. | |
| | A history of infection with human immunodeficiency virus | |
| | (HIV). | |
| | A history of active or latent tuberculosis (TB); TB testing | |
| | should be performed at screening and again during the | |
| | study, if clinically indicated, and may be repeated based on | |
| | clinical judgment, borderline results, or clinical suspicion | |
| | of TB infection. | |
| | NOTE: | |
| | The Investigator may consult with an infectious | |
| | disease expert if required, eg, test results are unclear or | |
| | there is a suspicion of false positive test results. If the | |
| | infectious disease expert considers the test results as false | |
| | positive and not clinically relevant and confirms that the | |
| | participant can be enrolled in the trial, the Investigator | |
| | must document this in the source data and may then | |
| | randomize the participant. | |
| | Persistent chronic or active or recurring system infection | |
| | that may adversely affect participation or IMP | |
| | administration in this study as judged by the investigator. | |
| | Fever within 4 weeks of the screening visit (≥38° C.; | |
| | however, if due to brief and mild ear, nose, throat viral | |
| | infection participant may be included based on the | |
| | Investigator's judgment). | |
| | Participants at risk of developing or having reactivation of | |
| | hepatitis: results at screening for serological markers for | |
| | hepatitis B and C indicating acute or chronic infection. See | |
| | the Study Manual for further details. | |
| | E 02. The presence of psychiatric disturbance or substance abuse | |
| | as evidenced by: | |
| | A history of any psychiatric disease, behavioral condition, | |
| | or depression requiring hospitalization within 2 years prior | |
| | to the screening visit. | |
| | A documented history of attempted suicide or suicidal | |
| | ideation of category 4 or 5 according to the Columbia | |
| | Suicide Severity Rating Scale (C-SSRS) | |
| | baseline/screening version over the 6 months prior to the | |
| | Screening Visit, OR if in the Investigator's judgment, the | |
| | participant is at risk for a suicide attempt. | |
| | A history of alcohol or drug abuse within 1 year prior to | |
| | the Screening Visit. | |
| | E 03. The following findings obtained during the Screening Visit | |
| | considered in the Investigator's judgment to be clinically | |
| | significant in the context of this trial: | |
| | Any screening laboratory values outside normal limits. | |
| | Abnormal ECG. | |
| | E 04. Any conditions that may predispose the participant to | |
| | excessive bleeding: | |
| | A bleeding disorder or known platelet dysfunction at any | |
| | time prior to the screening visit. | |
| | A platelet count <150 000/μL at the screening visit. | |
| | The participant has had major surgery within 4 weeks prior | |
| | to the screening visit, which could affect the participant's | |
| | safety (as judged by the Investigator) or has planned any | |
| | elective major surgery during the study. | |
| | A history of significant bleeding event within 6 months | |
| | prior to screening, according to the Investigator's judgment | |
| | such as, but not limited to cerebral or gastrointestinal | |
| | bleeding. | |
| | E 05. Conditions that would adversely affect participation in the | |
| | study or make the primary efficacy endpoint non- | |
| | evaluable: | |
| | A short life expectancy due to pre-existing health | |
| | condition(s) as determined by their treating neurologist. | |
| | A history or presence of significant other concomitant | |
| | illness according to the Investigator's judgment such as, | |
| | but not limited to cardiovascular (including Stage III or IV | |
| | cardiac failure according to New York Heart Association | |
| | [NYHA] classification), or renal (ie, undergoing dialysis), | |
| | neurological, endocrine, gastrointestinal, hepatic | |
| | (ie, underlying hepatobiliary disease, metabolic, | |
| | pulmonary, or lymphatic disease that would adversely | |
| | affect participation in this study. | |
| | Any malignancy within 5 years prior to the Screening Visit | |
| | (except for effectively treated carcinoma in situ of the | |
| | cervix or adequately treated non-metastatic squamous or | |
| | basal cell carcinoma of the skin) will also be exclusionary. | |
| Prior/concomitant | E 06. A requirement for concomitant treatment that could bias | |
| therapy | the primary evaluation, such as any of the following | |
| | medications/treatments within the specified time frame | |
| | before any randomization assessment: | |
| | Medication | Exclusionary if |
| | | used/used within |
| | | required wash- |
| | | out period |
| | High dose systemic corticosteroids | 1 month prior to randomization |
| | (pulse therapy) | |
| | Plasma exchange | 1 month prior to randomization |
| | IV immunoglobin | 1 month prior to randomization |

| | |
|---|---|
| | E 07. Participants using azathioprine, mycophenolate mofetil, |
| | methotrexate and other immunosuppressive medications |
| | will use 20 mg daily prednisone through the 4 week |
| | screening period while weaning off these background |
| | therapies. Prednisone will then be weaned down by 5 mg |
| | daily every week at the start of the 1-year treatment period, |
| | concurrent with use of 60 mg daily tolebrutinib. No |
| | additional prednisone or background immunotherapy is |
| | permitted during the 1-year trial period except as treatment |
| | for a confirmed relapse. |
| | E 08. The participant is receiving potent and moderate inducers |
| | of cytochrome P450 3A (CYP3A) or potent inhibitors of |
| | CYP2C8 hepatic enzymes. |
| | E 09. The participant is receiving anticoagulant/antiplatelet |
| | therapies, including: |
| | Acetylsalicylic acid (aspirin) >81 mg/day, |
| | Antiplatelet drugs (eg, clopidogrel), |
| | Warfarin (vitamin K antagonist), |
| | Heparin, including low molecular weight heparin |
| | (antithrombin agents), |
| | Dabigatran (direct thrombin inhibitor), |
| | Apixaban, edoxaban, rivaroxaban (direct factor Xa |
| | inhibitors). |
| | Note: |
| | All the above-mentioned drugs must be stopped at least 5 |
| | half-lives before study drug administration except for aspirin, |
| | which must be stopped at least 8 days before. If this is not |
| | clinically appropriate, the participant cannot be included. These |
| | washout periods are only applicable in the case that the |
| | Investigator deems it clinically appropriate to discontinue the |
| | listed medications or there is a recent history of use of these |
| | medications (as in the case when short term treatment with |
| | anticoagulants is clinically recommended for certain thrombotic |
| | events), and therefore these washout periods will need to be |
| | followed prior to randomization. |
| | If the participant has a chronic underlying medical condition |
| | (stroke, coronary or carotid artery disease, heart valvular disease |
| | etc.) requiring continued use of these medications, the participant |
| | cannot be enrolled in the study. |
| | E 10. The participant has sensitivity to any of the study |
| | interventions, or components thereof, or has a drug or other |
| | allergy that, in the opinion of the Investigator, |
| | contraindicates participation in the study. |
| Prior/concurrent | E 11. The participant was previously exposed to any BTK |
| clinical study | inhibitor, including tolebrutinib. |
| experience | E 12. The participant has taken other investigational drugs within |
| | 3 months or 5 half-lives, whichever is longer, before the |
| | screening visit. |
| Diagnostic | E 13. The participant has a contraindication for MRI, ie, presence |
| assessments | of pacemaker, metallic implants in high-risk areas (ie, |
| | artificial heart valves, aneurysm/vessel clips), presence of |
| | metallic material (eg, shrapnel) in high risk areas, known |
| | history of allergy to any contrast medium, or history of |
| | claustrophobia that would prevent completion of all |
| | protocol scheduled MRI. |
| | Note: |
| | People with a contraindication to Gd can be enrolled into |
| | the study but cannot receive Gd contrast dyes during their MRI |
| | scan. |
| Other exclusions | E 14. Individuals accommodated in an institution because of |
| | regulatory or legal order; prisoners or participants who are |
| | legally institutionalized |
| | E 15. Participant not suitable for participation, whatever the |
| | reason, as judged by the Investigator, including medical or |
| | clinical conditions, or participants potentially at risk of |
| | noncompliance to study procedures |
| | E 16. Participants are employees of the clinical study site or |
| | other individuals directly involved in the conduct of the |
| | study, or immediate family members of such |
| | individuals (in conjunction with section 1.61 of the ICH- |
| | GCP Ordinance E6) |
| | E 17. Any specific situation during study implementation/course |
| | that may raise ethics considerations |
| | E 18. Sensitivity to any of the study interventions, or components |
| | thereof, or drug or other allergy that, in the opinion of the |
| | Investigator, contraindicates participation in the study |
| | Note: |
| | a one-time retest at screening may be performed if an |
| | abnormal laboratory test value is considered temporary |

### Example 1.3C—Lifestyle Considerations

Meals and dietary restrictions: tolebrutinib (IMP) shall be taken with a regular meal. When possible, the meal with which IMP is taken (eg, breakfast, lunch, or dinner) should be consistent throughout the study. The typical meal with which IMP is taken will be collected at each visit. In case the mealtime needs to be changed for JIMP administration, a gap of a minimum of 12 hours between 2 doses should be maintained.

### Example 1.4—Study Intervention(s) and Concomitant Therapy

Study intervention is defined as any investigational intervention(s), marketed product(s), placebo, or medical device(s) intended to be administered to a study participant according to the study protocol.

### Example 1.4A—Study Intervention(s) and Concomitant Therapy Administered

This study intervention includes an IMP and a noninvestigational medicinal product (NIMIP). To maintain blinding, participants receive 4 tablets once per day of the BTK inhibitor or placebo in a blinded fashion. Details for the interventions are provided in Table 5.

TABLE 5
Overview of study interventions administered

| | |
|---|---|
| Intervention label | tolebrutinib |
| Intervention name | tolebrutinib 60 mg |
| Type | Drug |
| Dose formulation | Film coated tablet |
| Unit dose strength(s) | 60 mg |
| Dosage level(s) | Once daily |
| Route of administration | Oral |
| Use | Experimental |
| IMP or NIMP | IMP |
| Packaging and labeling | Study Intervention will be provided |
| | in wallet blister packing. Each package |
| | will be labeled as required per country |
| | requirement. |
| Current name | tolebrutinib |

*IMP: investigational medicinal product;
NIMP: noninvestigational medicinal product

Details of noninvestigational medicinal products are provided in Table 6.

TABLE 6
Overview of Noninvestigational Medicinal Products

| | |
|---|---|
| MRI contrast- | Route(s) of administration: IV. |
| enhancing preparations | Dose regimen: as per respective label. |
| (only in the case of a | |
| suspected relapse) | |
| Prednisone | Route(s) of administration: Oral. |
| | Dose regimen: 20 mg daily prednisone for 4 |
| | weeks during the screening period, followed |
| | by weekly weaning by 5 mg daily for an |
| | additional 4 weeks at the start of the |
| | treatment period. |
| Relapse treatment | Option #1 |
| recommendations: | Formulation: Methylprednisone 1000 mg. |
| | Route(s) of administration: Intravenous. |
| | Dose regimen: Daily methylprednisone for 3-7 |
| | consecutive days upon confirmation of |
| | inflammatory MOG antibody disease relapse. |
| | Oral prednisone taper of 8 weeks follows |
| | completion of intravenous therapy starting at |
| | 80 mg daily with decreases in the daily dose |
| | by 10 mg every week. |
| | Option #2 |
| | Formulation: 10% Human purified |
| | immunoglobulin 2 grams/kg of ideal body weight. |
| | Route(s) of administration: Intravenous. |
| | Dose regimen: Up to 5 daily infusions of 0.4 |
| | grams/kg upon confirmation of inflammatory |
| | MOG antibody disease relapse. |
| | No prednisone taper required or recommended. |

### Example 1.4B—Measures to Minimize Bias: Randomization and Blinding

This is an open-label study; potential bias will be reduced by the following steps: Objective measures will be used to determine the primary outcome of relapses. Relapses are defined clinically, but also require MRI confirmation of the attack that explains the clinical symptoms.

### Example 1.4C—Dose Modification

Dose modification is not foreseen in this study. Treatment may need to be interrupted or permanently discontinued if deemed necessary due to an AE.

### Example 1.4D—Continued Access to Intervention after the End of the Study

Post trial access may be considered if mandated by local regulations.

### Example 1.4E—Concomitant Therapy

Any medication or vaccine (including over-the-counter or prescription medicines, vitamins, or herbal supplements) that the participant is receiving at the time of enrollment or receives during the study must be recorded along with: Reason for use; Dates of administration including start and end dates; Dosage information including dose and frequency.

Live (attenuated) vaccines should not be administered during the intervention period.

Therapies for MOG antibody disease noted in the exclusion criterion Example 1.3B are not permitted after randomization while the participant is on study treatment. Short-term use (3 to 7 days) of glucocorticoids (eg, for relapse treatment or an acute illness) and local corticosteroids (eg, topical, nasal, ocular, otic, intra-articular) are allowed.

The Medical Monitor should be contacted if there are any questions regarding concomitant or prior therapy.

Participants must abstain from taking prescription or nonprescription drugs (including vitamins and dietary or herbal supplements) within 7 days (or 14 days if the drug is a potential enzyme inducer) or 5 half-lives (whichever is longer) before the start of study intervention until completion of the follow-up visit, unless, in the opinion of the Investigator and Sponsor, the medication will not interfere with the study.

Medications for the treatment of MOG antibody disease symptoms (eg, walking impairment, spasticity, incontinence, pain) should be maintained at a stable dose prior to screening and for the duration of the treatment period, if clinically feasible.

Anticoagulant/antiplatelet therapies are not permitted to be taken concomitantly with the IMP, including: Acetylsalicylic acid (aspirin)>81 mg/day; Antiplatelet drugs (eg, clopidogrel); Warfarin (vitamin K antagonist); Heparin, including low molecular weight heparin (antithrombin agents); Dabigatran (direct thrombin inhibitor); Apixaban, edoxaban, rivaroxaban (direct factor Xa inhibitors).

Paracetamol/acetaminophen, at doses of ≤3 grams/day, is permitted for use at any time during the study. Short courses (up to 5 days) of nonsteroidal anti-inflammatory drugs (NSAIDs) (other than acetylsalicylic acid), preferably selective cyclooxygenase-2 inhibitors such as NSAIDs at the lowest effective dose, may be given during the study if clinically necessary for the treatment of an existing medical condition or a new event. The Investigator must record the use of NSAIDs (and any other comedication) in the eCRF.

CYP Inhibitor/Inducer: Potent and moderate inducers of CYP3A or potent inhibitors of CYP2C8 hepatic enzymes are not allowed throughout the study.

### Example 1.5—Discontinuation of Study Intervention and Participant Discontinuation/Withdrawal

### Example 1.5A—Discontinuation of Study Intervention

### Example 1.5A1—Permanent Discontinuation

The study intervention should be continued whenever possible. Permanent intervention discontinuation is any treatment discontinuation associated with the definitive decision by the Investigator or the participant not to re-expose the participant to study intervention at any time. In rare instances, it may be necessary for a participant to permanently discontinue study intervention. If study intervention is permanently discontinued, the participant shall be asked to remain in the study to be evaluated until the follow-up visit. For this set of participants (participants who discontinued the IMP or switched to another immunomodulatory/immunosuppressive drug), no biomarker samples will be collected after the premature end of treatment visit. This will be important to continue to evaluate for safety. See the SoA (Example 1.1) for data to be collected at the time of discontinuation of study intervention. In the case that the study intervention is permanently discontinued, the participant should be treated for MOGAD according to local clinical practice and the best judgment of the Treating Investigator.

The following may be justifiable reasons for the Investigator to discontinue a participant from study treatment:
Adverse events that endanger the safety of the participant, or if discontinuation of study intervention is desired or considered necessary by the Investigator or participant.
If IMP discontinuation criteria are met as per the guidance for the follow up of laboratory abnormalities (Example 1.8A).
The participant is no longer deriving a therapeutic/clinical benefit in the opinion of the Investigator.
At participant's request, ie, withdrawal of the consent for treatment.
If a female participant becomes pregnant or wishes to become pregnant during the study.
Any serious opportunistic infections (eg, PML (Example 1.8C), HIV).
Continued need for chronic use of a prohibited concomitant medication.

Discontinuation of study intervention for abnormal liver function should be considered by the Investigator when a participant meets one of the conditions outlined in the algorithm (Example 1.8C) or if the Investigator believes that it is in the best interest of the participant.

Any clinically significant abnormal laboratory value or ECG parameter will be immediately rechecked for confirmation after 24 hours before making a decision of definitive discontinuation of the IMP for the concerned participant.

If a clinically significant finding is identified in the ECG (including, but not limited to changes from baseline in QT interval corrected using Fridericia's formula [QTcF]) after enrollment, the Investigator or qualified designee will determine if the participant can continue in the study and if any change in participant management is needed. Review of ECG findings by a cardiologist may be considered for a decision of a definitive discontinuation of study intervention because of ECG changes. This review of the ECG printed at the time of collection must be documented. Any new clinically relevant finding should be reported as an AE. See the SoA (Example 1.1) for data to be collected at the time of intervention discontinuation and follow up and for any further evaluations that need to be completed.

Handling of participants after permanent intervention discontinuation: Participants will be followed according to the study procedures specified in this protocol:
Up to the scheduled date of study completion, or
Up to recovery or stabilization of any AE to be followed as specified in this protocol, whichever comes last.

The participant should be treated for MOG antibody disease according to local clinical practice and the best judgement of the Treating Investigator.

If possible, and after the definitive discontinuation of study drug, the participants will be assessed using the procedures planned for the premature end of treatment visit.

Participants will be asked to continue in the study, attending all scheduled visits per SoA (Example 1.1), if possible until the end of the study. If the participant does not agree to the full visit schedule after a decision for permanent end of treatment, a reduced visit schedule may be agreed with the participant. Every effort should be made to collect endpoint information and vital status as frequent as possible.

For participants with definitive discontinuation of IMP who do not agree to remain in the study after the premature end of treatment visit, if the premature end of treatment visit is less than 3 weeks after the last dose of the study drug, an additional visit should be performed with assessments normally planned for the follow-up visit.

Patients who are treated with a non-study drug who decide to prematurely and permanently discontinue study participation prior to their study end date should be assessed as soon as possible using the procedures normally planned for the premature end of treatment visit. Participants who prematurely and permanently discontinue the study drug who do not agree to remain in the study after performing a visit analogous to the premature end of treatment visit, should have an additional visit performed with assessments normally planned for the follow-up visit if the last study visit was less than 3 weeks after the last dose of the open label IMP.

All cases of definitive study drug discontinuation must be recorded by the Treating Investigator in the appropriate pages of the eCRF when considered as confirmed.

### Example 1.5A2—Liver Chemistry Stopping Criteria

Discontinuation of study intervention for abnormal liver tests is required by the Investigator when a participant meets one of the conditions outlined in Example 1.8C or in the presence of abnormal liver chemistries not meeting protocol-specified stopping rules if the Investigator believes that it is in best interest of the participant.

### Example 1.5A3—QTc Stopping Criteria

If a clinically significant finding is identified (including, but not limited to changes from baseline in QT interval corrected using Fridericia's formula [QTcF] after enrollment, the Investigator or qualified designee (eg, local physician) will determine if the participant can continue in the study and if any change in participant management is needed. This review of the ECG printed at the time of collection must be documented. Any new clinically relevant finding should be reported as an AE.

### Example 1.5A4—Temporary Discontinuation

Temporary intervention discontinuation because of suspected AEs or disruption of the clinical trial due to a regional or national emergency declared by a governmental agency: Contingency measures for a regional or national emergency that is declared by a governmental agency) may be considered by the Treating Investigator. For all temporary intervention discontinuations, duration should be recorded by the Investigator in the appropriate pages of the eCRF.

If surgery is needed during the study, consider the benefit-risk of withholding the IMP for at least 3 to 7 days pre- and post-surgery depending upon the type of surgery and the risk of bleeding.

The following shall lead to temporary treatment discontinuation:
Cytopenia: follow Sanofi algorithm for neutropenia and thrombocytopenia as per Example 1.8C.
Serum creatinine, creatine phosphokinase (CPK) and liver enzyme increase: follow corresponding Sanofi algorithms as per Example 1.8C.
Cardiac arrhythmia (atrial fibrillation): Any Grade 3 event (symptomatic, urgent intervention indicated; device [eg, pacemaker]; ablation; new onset).
Suicidal risk as per C-SSRS: if a participant scores “yes” on items 4 or 5 of the Suicidal Ideation Section, or “yes” on any item of the Suicidal Behavior Section.

If needed, temporary treatment discontinuation can be considered by the Investigator or by the participant for any other reason, including due to any safety concerns because of disruption of the clinical trial due to a regional or national emergency declared by a governmental agency such as COVID-19 or another illness or if there is a need for a prohibited concomitant medication. Treatment can be resumed later when it is considered safe and appropriate.

### Example 1.5A5—Rechallenge

Reinitiation of the IMP will be done under close and appropriate clinical/and or laboratory monitoring once the Investigator will have considered according to his/her best medical judgment that the responsibility of the IMP(s) in the occurrence of the concerned event was unlikely, there are no safety concerns and if the criteria for permanent treatment discontinuation have not been met.

### Example 1.5A6—Study Intervention Restart or Rechallenge after Liver Stopping Criteria Met

Study intervention restart or rechallenge after liver chemistry stopping criteria are met by any participant in this study is not allowed.

### Example 1.6—Study Assessments and Procedures

Study procedures and their timing are summarized in the SoA (Example 1.1). Protocol waivers or exemptions are not allowed.

Adherence to the study design requirements, including those specified in the SoA (Example 1.1), is essential and required for study conduct.

All screening evaluations must be completed and reviewed to confirm that potential participants meet all eligibility criteria. The Investigator will maintain a screening log to record details of all participants screened and to confirm eligibility or record reasons for screening failure, as applicable.

Procedures conducted as part of the participant's routine clinical management (eg, blood count) and obtained before signing of the ICF may be utilized for screening or baseline purposes provided the procedures met the protocol-specified criteria and were performed within the time frame defined in the SoA (Example 1.1). Such assessments shall be recorded on the designated field in the CRF.

Blood sampling details including volume for all laboratory assessments will be provided in the informed consent form. Repeat or unscheduled samples may be taken for safety reasons or for technical issues with the samples.

### Example 1.6A—Efficacy Assessments

Planned time points for all efficacy assessments are provided in the SoA (Example 1.1).

Key efficacy assessments (eg, EDSS) will be scored by the study team.

The Investigator will be responsible for the confirmation of relapses. MRI findings and local neurologist assessment during the acute attack will be needed in these cases.

The MRI scans for efficacy analyses (relapse evaluation and follow-up relapse visit) will be analyzed by the Investigator.

The basic MRI scan will be performed locally and will consist of the following sequences: T2- and T1-weighted sequences before and after administering a Gd contrast (if there is no contraindication).

### Example 1.6A1—Definition of MOG Antibody Disease Relapse

For the purposes of this study, MOG antibody disease relapse is defined as a monophasic, acute or subacute onset of, new neurological symptoms or worsening of previous neurological symptoms with an objective change on neurological examination. Symptoms must:
Be attributable to MOG antibody disease,
Last for ≥24 hours, with or without recovery,
Be present at normal body temperature (ie, no infection, excessive exercise, or excessively high ambient temperature),
Be preceded by ≥30 days of clinical stability (including no previous relapse), and
Be attributable to a new or enhancing MRI lesion(s) in the brain or the spinal cord or the optic nerves.
   Note: An exacerbation or recurrence of symptoms and signs in a participant with MOG antibody disease that can be reasonably attributed to transient impairment of conduction in previously demyelinated pathways due to raised core body temperature will not be considered a relapse.

Confirmation of MOG antibody disease relapse will be done by the Investigator based on the following definition:
A confirmed MOG antibody disease relapse is one accompanied by a clinically relevant change in the EDSS score performed by the local neurologist, ie, an increase of at least 0.5 points in the EDSS score, an increase of 1 point on 2 functional scores, or an increase of 2 points on 1 functional score, excluding changes involving bowel/bladder and cerebral functional score compared to the previously available rating (the last EDSS rating that did not occur during a relapse).
MRI (brain or spinal cord or optic nerves) must be performed as soon as possible to support the confirmation of a relapse.

### Example 1.6A2—Relapse Evaluation and Follow-Up Relapse Visits

Participants must be instructed to immediately report new neurological symptoms and recurring or worsening of previous symptoms to the Investigator. Any reported symptoms will be collected. If a participant reports symptoms that may be consistent with relapse, a relapse evaluation visit with the Investigator must be scheduled as soon as possible (whenever possible within 7 days of onset of the symptoms). The assessment and reporting of MOG antibody disease relapse is made by the Investigator. The management of the relapse will be done locally by the treating neurologist according to local standard of care. Recommendations for the treatment of relapses are detailed in Table 6 and are not mandatory.

Diagnosing MOG antibody disease relapses during the study: The local neurologist will assess whether the reported episode is consistent with the definition of relapse, which includes MRI findings as well. If it is consistent with the definition of MOG antibody disease relapse or if there is any doubt and possibility of relapse cannot be ruled out, the standard neurological examination (for the EDSS score) will be performed by the local neurologist and the case will be discussed with the Investigator. If the participant is not referred for EDSS assessment, this will be documented with an explanation of the reason. Whenever possible, the local neurologist should perform the EDSS rating and at least request the MRI before the relapse evaluation visit is performed by the Investigator.

All MOG antibody disease relapses must be reported on the relapse eCRF page. MOG antibody disease relapse should not be reported as an AE unless, in the judgment of the Investigator, it is unusually severe or medically unexpected, or matches definition of an SAE.

Safety laboratory tests are optional for this relapse evaluation visit if no intercurrent disease is suspected. If any intercurrent disease is diagnosed, it will be reported as an AE as per the safety reporting rules.

The participant will be actively asked about possible relapse symptoms at each study visit. If relapse is suspected, the above decision-making and reporting rules apply.

A follow-up relapse visit will be performed by the Investigator 4 weeks after a confirmed relapse as per the SoA (Example 1.1).

### Example 1.6B—Safety Assessments

Planned time points for all safety assessments are provided in the SoA (Example 1.1).

### Example 1.6B1—Electrocardiograms

12-lead ECG will be obtained as outlined in the SoA (Example 1.1) using an ECG machine that automatically calculates the heart rate and measures PR, QRS, QT, and QTcF intervals. In case the ECG machine does not automatically calculate QTcF, manual calculation using nomogram or automatic website calculator (eg, https.//reference.medscape.com/calculator/48/ecg-corrected-qt) is acceptable.

ECGs and 30-second rhythm strips will be obtained locally.

### Example 1.6B2—Vital Signs

Body temperature, heart rate, and blood pressure will be assessed.

Blood pressure and heart rate measurements will be assessed with the participant in a supine or sitting position with a completely automated device. Manual techniques will be used only if an automated device is not available.

Blood pressure and heart rate measurements should be preceded by at least 5 minutes of rest for the participant in a quiet setting without distractions (eg, television, cell phones).

Heart rate and blood pressure measurements will be taken before blood collection for laboratory tests and will consist of 1 heart rate and 3 blood pressure measurements (3 consecutive blood pressure readings will be recorded at intervals of at least 1 minute).

### Example 1.6B3—Clinical Safety Laboratory Assessments

See Example 1.8A for the list of clinical laboratory tests to be performed. Per the SoA (Example 1.1), serology tests for hepatitis B and C will be performed during screening; testing for other infectious diseases should be performed during screening if required locally.

The Investigator may solicit emergency local laboratory data in case of emergent safety events to allow for appropriate treatment decisions. All clinically relevant solicited emergency local laboratory data will be recorded in the eCRF.

The Investigator must review the laboratory report, document this review, and record any clinically relevant changes occurring during the study in the AE section of the eCRF. The laboratory reports must be filed with the source documents. Clinically significant abnormal laboratory findings are those which are not associated with the underlying disease, unless judged by the Investigator to be more severe than expected for the participant's condition.

All laboratory tests with values considered clinically significantly abnormal during participation in the study or within 28 days after the last dose of study intervention should be repeated until the values return to normal or baseline or are no longer considered clinically significant by the Investigator.

If abnormal laboratory test values do not return to normal or baseline within a period of time judged reasonable by the Investigator, the etiology should be identified, and the Sponsor notified.

All protocol-required laboratory assessments, as defined in Example 1.8A must be conducted in accordance with the SoA. In the event the laboratory assessments in Example 1.8C indicate discontinuation of IMP, temporary discontinuation should be considered unless otherwise specified.

If laboratory values from non-protocol-specified laboratory assessments performed at the institution's local laboratory require a change in participant management or are considered clinically significant by the Investigator (eg, SAE or AE or dose modification), then the results must be recorded in the eCRF.

### Example 1.6C—Adverse Events (AEs), Serious Adverse Events (SAES) and Other Safety Reporting

### Example 1.6C1—MOG Disease Relapse Reporting

MOG disease relapses, determined from the evaluations described in Example 1.5A1, will be exempt from being reported as AEs except when they meet the definition of a SAE, or are unusually severe or medically unexpected. Hospitalization for a relapse, if done routinely at the site (eg, for high dose IV methylprednisolone), will not be considered as a seriousness criterion for this study.

Data for MOG disease relapses will be collected on the eCRF and be analyzed as part of the efficacy analysis. Other worsening of neurological symptoms that do not meet the definition of MS relapse will be reported as AEs according to general safety reporting rules.

### Example 1.6C2—Reporting of Safety Findings from Magnetic Resonance Imaging

Magnetic resonance imaging scans need to be reviewed locally for any pathology. In case of clinically relevant findings, relevant information needs to be provided to the Investigator for appropriate safety reporting and to ensure the appropriate management of the participant's identified safety finding. When available, a diagnosis of pathology as a cause of such MRI findings or the findings themselves will be reported as an AE until the diagnosis is clear.

### Example 1.6C3—Adverse Event of Special Interest

An AE of special interest (AESI) is an AE (serious or nonserious) of scientific and medical concern specific to the Sponsor's product or program, for which ongoing monitoring and immediate notification by the Investigator to the Sponsor is required. Such events may require further investigation in order to characterize and understand them. Adverse events of special interest may be added, modified, or removed during a study by protocol amendment.

Pregnancy of a female participant entered in a study as well as pregnancy occurring in a female partner of a male participant entered in a study with IMP/NIMP.
It will be qualified as an SAE only if it fulfills one of the seriousness criteria (see Example 1.8B).
In the event of pregnancy in a female participant, the IMP should be discontinued.
Follow up of the pregnancy in a female participant or in a female partner of a male participant is mandatory until the outcome has been determined.

Symptomatic overdose (serious or nonserious) with IMP.
An overdose (accidental or intentional) with the IMP is an event suspected by the Investigator or spontaneously notified by the participant (not based on systematic pills count) and defined as at least twice the intended dose within the intended therapeutic interval (eg, >2 tablets of the IMP within a 12-hour interval).

Increase in alanine transaminase (ALT)>3×ULN.
ALT increase>3×ULN confirmed by retest within 72 hours or in the absence of a retest within 72 hours.

Project specific AESI(s) are:
ECG observation of atrial fibrillation or atrial flutter
Severe infection (Grade 3 or above by National Cancer Institute Common Terminology Criteria for Adverse Event [NCI CTCAE]), that may or may not meet seriousness criteria (eg, a Grade 3 opportunistic infection).
Moderate or severe hemorrhagic events (NCI CTCAE Grade 2 or above), including but not limited to symptomatic bleeding in a critical area or organ such as the CNS, or intraocular bleeding.
Thrombocytopenia, platelet count<75,000/mm³ (see Example 1.8C for the management flow chart).

The definitions of an AE or SAE can be found in Example 1.8B.

Adverse events will be reported by the participant (or, when appropriate, by a caregiver, surrogate, or the participant's legally authorized representative).

The Investigator and any qualified designees are responsible for detecting, documenting, and recording events that meet the definition of an AE or SAE and remain responsible for following up AEs that are serious, considered related to the study intervention or study procedures, or that caused the participant to discontinue the study intervention.

### Example 1.6D—Biomarkers

Blood samples for biomarkers research will be collected from all participants in this study as specified in the SoA (Example 1.1). Participants who have had a relapse during the course of the trial will have the option to collect blood samples for biomarkers from that point on.

Collection of plasma and serum samples for biomarker research is also part of this study.

Samples will be tested to evaluate biomarkers and their association with the observed clinical responses to tolebrutinib.

In addition, samples will be stored, and analysis may be performed on biomarker variants thought to play a role in MOG antibody disease including, but not limited to, serum analytes to evaluate their association with observed clinical responses to tolebrutinib (in accordance with local regulations).

Samples may be stored for a maximum of 2 years (or according to local regulations) following the last participant's last visit for the study at a facility selected by the Investigator to enable further analysis of biomarker responses to tolebrutinib.

### Example 1.7—Statistical Considerations

### Example 1.7A—Sample Size Determination

Approximately 25 participants will be enrolled to study intervention. This is a pilot, single arm, exploratory study with little existing data in MOG antibody disease patients on which to base a sample size calculation. Clinically, it is estimated that MOG antibody disease patients have a relapse once every year on average. If we assume that 60% of patients would have at least one relapse in a 1-year period (i.e., 40% relapse free), 25 patients provides 80% power to detect a difference between the null and alternative hypotheses, H0: π=0.4 and H1: π≥0.66, where π denotes the proportion of relapse free patients at the end of the 1 year treatment period; based on an exact binomial test with a nominal 5% one-sided significance level. We would reject the null hypothesis that tolebrutinib is not efficacious in preventing relapse in MOG antibody disease if at least 14 of the 25 patients enrolled and treated patients in the current study are relapse free at 1 year.

### Example 1.7B—Populations for Analyses

The following populations for analyses are defined as shown in Table 7.

TABLE 7
Populations for analyses

| Population | Description |
|---|---|
| Screened | All participants who signed the ICF. |
| Exposed | All screened participants who take at least 1 dose |
| | of study intervention. |
| Enrolled | All participants from screened population who have |
| | been allocated to the intervention regardless of |
| | whether the intervention was received or not. |
| Per | All enrolled and treated participants who complete |
| protocol | 1 year tolebrutinib treatment or experience relapse. |
| (PP) | Participants who prematurely discontinue study |
| | treatment for reasons other than relapse will be |
| | excluded. This will be the primary population used |
| | for efficacy. |
| Safety | All enrolled participants who take at least 1 dose |
| | of study intervention. This will be the population |
| | used for safety and also as a secondary population |
| | for efficacy. |

Participants exposed to study intervention before or without being enrolled will not be considered enrolled and will not be included in any analysis population. The safety experience of these participants will be reported separately.

For any participant enrolled more than once, only the data associated with the first enrollment will be used in any analysis population. The safety experience associated with any later enrollment will be reported separately.

### Example 1.7C—Statistical Analyses

The statistical analysis plan (SAP) will include a more technical and detailed description of the statistical analyses described in this section. This section is a summary of the planned statistical analyses of the most important endpoints including primary and key secondary endpoints. Given this is a single arm efficacy trial, the majority of the data will be expressed as descriptive statistics.

### Example 1.7C1—General Considerations

The baseline value is defined as the last available value before the first dose of IMP. For participants enrolled but not treated, the baseline value is defined as the last available value before enrollment. Baseline EDSS will be calculated as the average of the screening and day 1 assessment values.

The observation period will be divided into 3 segments:
The pre-treatment period is defined as the period from signed ICF up to first IMP administration.
The treatment-emergent (TE) period is defined as the period from the first IMP administration to the last IMP administration+10 days.
The post-treatment period is defined as the period from the end of the treatment-emergent period to the last study assessment for the participant.

### Example 1.7C2—Primary Endpoint(s)

The point estimate and exact 95% confidence interval for the proportion of participants free of relapse at 1 year in the PP population will be provided. The observed success rate will aid in the decision to continue development of tolebrutinib in MOG antibody disease or not.

Time to relapse after enrollment will be calculated and a Kaplan-Meier (KM) plot of the cumulative incidence rate will be provided to depict the course of onset of relapse over time in the PP population. The proportion of participants with relapse at 3, 6, 9 and 12 months will be calculated using the KM estimates. Participants who complete treatment without relapse will be censored at their end of treatment date.

Sensitivity analyses including participants that prematurely discontinue study treatment for reasons other than relapse will be provided where:
All these participants are censored at their date of premature treatment discontinuation.
Participants, if any, who experience relapse in the 10 days after premature discontinuation of tolebrutinib are included as having an event and the others are censored at their date of premature treatment discontinuation+10 days.

Time to relapse on tolebrutinib compared to time to relapse in the same group of participants in the year after initiation of their first treatment for MOG antibody disease will be explored, though those prior treatments will be variable. Furthermore, time to relapse on tolebrutinib compared to time to relapse in the year after first treatment initiation for MOG antibody disease in a propensity score matched external control arm will also be investigated. Crude KM curves and estimates will be provided. Various statistical methods will be explored to provide estimates of the reduction in the probability of a relapse on tolebrutinib compared to “other treatment.” Details will be provided in the SAP.

### Example 1.7C3—Secondary Endpoint(s)

EDSS and MOG antibody titers change from baseline after 1 year of tolebrutinib treatment will be summarized for secondary efficacy endpoints in the PP population without relapse.

### Example 1.7C4—Tertiary/Exploratory Endpoint(s)

Details for any tertiary/exploratory endpoints will be included in the SAP.

### Example 1.7C5—Safety Analysis

All safety analyses will be performed on the Safety Population.

### Example 1.7C6—Adverse Events

General common rules for adverse events: AE summaries will be provided depending on the number of events. In case of small numbers, listings of events will be provided.

The AEs will be analyzed in the following 3 categories:
Pre-treatment AEs: AEs that developed, worsened or became serious during the pre-treatment period.
TEAEs: AEs that developed, worsened or became serious during the treatment-emergent period.
Post-treatment AEs: AEs that developed, worsened or became serious during the post-treatment period.

Information for any deaths will be listed.

Analysis of all adverse events: Adverse event incidence table will be provided for all types of TEAEs: all TEAEs, all treatment emergent AESI (defined with a PT or a prespecified grouping), all treatment emergent SAEs and all TEAEs leading to permanent treatment discontinuation.

The AE summaries will be generated with number (%) of participants experiencing at least one event.

Deaths will also be analyzed.

### Example 1.7C7—Laboratory Variables, Vital Signs and Electrocardiograms (ECGs)

Quantitative analyses: For laboratory variables, vital signs and ECG variables, descriptive statistics for results and changes from baseline will be provided for each planned visit during the on-treatment period. These analyses will be performed using central measurements for MOG antibody titers and other laboratory variables and local measurements for vital signs, and ECG variables.

Analyses of laboratory, ECG and vital signs abnormalities: Potentially clinically significant laboratory, ECG and vital signs abnormalities will be summarized.

### Example 1.7C8—Other Safety

Summaries of participants for the C-SSRS categories of suicidal ideation or suicidal behavior will be provided.

### Example 1.7C9—Other Analysis

Biomarker exploratory analyses that will be included in the study report will be described in the SAP.

### Example 1.7C10—Interim Analyses

There is no interim analysis planned for this study.

### Example 1.8—Supporting Documentation and Operational Considerations

### Example 1.8A—Clinical Laboratory Tests

The tests detailed in the table below in Table 8 will be performed by the central laboratory, when feasible. Local laboratory results are only required in the event that the central laboratory results are not available in time for either study intervention administration and/or response evaluation. Additionally, if the local laboratory results are used to make either a study intervention decision or response evaluation, the results must be entered into the eCRF.

Protocol-specific requirements for inclusion or exclusion of participants are detailed in Tables 3 and 4.

Additional tests may be performed at any time during the study as determined necessary by the Investigator or required by local regulations. Additional serum or urine pregnancy tests may be performed, as determined necessary by the Investigator or required by local regulation, to establish the absence of pregnancy at any time during the subject's participation in the study.

TABLE 8
Protocol-required laboratory assessments

| Laboratory assessments | Parameters | | |
|---|---|---|---|
| Hematology | Platelet count | RBC indices: | WBC count with differential: |
| | RBC count | MCV | Neutrophils |
| | Hemoglobin | MCH | Lymphocytes |
| | Hematocrit | % Reticulocytes | Monocytes |
| | | | Eosinophils |
| | | | Basophils |
| Clinical chemistry^{a} | BUN | Sodium | AST |
| | Creatinine | Calcium | ALT |
| | Glucose | Total and direct bilirubin | Alkaline phosphatase |
| | Potassium | Total protein | Albumin |
| | | Chloride | Creatine phosphokinase |
| | | Bicarbonate | Lipase |

| | |
|---|---|
| Routine urinalysis | Specific gravity |
| | pH, glucose, protein, blood, ketones, bilirubin, urobilinogen, |
| | nitrite, leukocyte esterase by dipstick |
| | Microscopic examination (if blood or protein is abnormal and |
| | for signs of infection) |
| Other screening | Serum anti-aqauaporin 4 antibody |
| tests | Serum anti-MOG antibody (screening and M12, if no relapses |
| | occur during the course of the trial) |
| | FSH (if needed, only in female participants to confirm |
| | postmenopausal state) |
| | Highly sensitive serum or urine β-hCG pregnancy test (as |
| | needed for women of childbearing potential)^{b} |
| | Coagulation: PT/ INR, aPTT |
| | Serology tests for hepatitis B (HBs Ag, anti-HBc IGM and total, |
| | anti-HBs) and C virus (anti-HCV); in case these results are |
| | inconclusive (eg anti-HBs negative and anti-HBc positive or |
| | anti-HC IgG positive), HBV-DNA or HCV-RNA testing, |
| | respectively, should be performed for confirmation. HIV and |
| | other infectious diseases, if locally required. |
| | Tuberculosis test: Blood testing (eg, QuantiFERON ® TB Gold |
| | test) is preferred; skin testing (eg, tuberculin skin test) with |
| | ancillary testing will be allowed if blood testing is not available. |
| | T-SPOT can also be performed, if available.^{c} |

ALT: alanine aminotransferase;
anti-HBc; antibody to hepatitis B core antigen;
anti-HBs: hepatitis B surface antibody;
aPTT: activated partial thromboplastin time;
AST: aspartate aminotransferase;
BUN: blood urea nitrogen;
β-hCG: human chorionic gonadotropin;
FSH; follicle-stimulating hormone;
IEC: independent ethics committee;
INR: international normalized ratio;
HBsAg: hepatitis B surface antigen;
HBV: hepatitis B virus;
HCV: hepatitis C virus;
HIV: human immunodeficiency virus;
Ig: immunoglobulin;
IRB: institutional review board;
MCH: mean corpuscular hemoglobin;
MCV: mean corpuscular volume;
PT: prothrombin time;
RBC: red blood cell;
SGOT: serum glutamic-oxaloacetic transaminase;
SGPT: serum glutamic-pyruvic transaminase;
TB: tuberculosis;
ULN: upper limit of normal;
WBC: white blood cell
^{a}Details of liver chemistry stopping criteria and required actions and follow-up assessments after observations of ALT >3 × ULN are given in Example 1.8C. Clinical laboratory findings of ALT >3 × ULN and bilirubin ≥2 × ULN (>35% direct bilirubin), or ALT >3 × ULN and international normalized ratio (INR) >1.5, if INR measured, that may suggest severe liver injury and must be reported as an SAE
^{b}Local urine testing for pregnancy will be standard for the protocol (except for the Screening Visit, when a serum pregnancy test is required) unless serum testing is required by local regulation or IRB/IEC.
^{c}Further details are provided in E 01, Table 4

Biomarkers (blood/serum): neurofilament light chain, glial fibrillary acidic protein level, tau, S100B and B/T cell profiling (potentially in a future analysis). Optional for participants who have relapsed during the course of the trial.

Investigators must document their review of each laboratory safety report.

### Example 1.8B—AEs and SAEs: Definitions and Procedures for Recording, Evaluating, Follow-Up, and Reporting

### Example 1.8B1—Definition of AE

### Example 1.8B1a—AE Definition

An AE is any untoward medical occurrence in a patient or clinical study participant, temporally associated with the use of study intervention, whether or not considered related to the study intervention.

NOTE: An AE can therefore be any unfavorable and unintended sign (including an abnormal laboratory finding), symptom, or disease (new or exacerbated) temporally associated with the use of study intervention.

### Example 1.8B1b—Events Meeting the AE Definition

Any abnormal laboratory test results (hematology, clinical chemistry, or urinalysis) or other safety assessments (eg, ECG, radiological scans, vital signs measurements), including those that worsen from baseline, considered clinically significant in the medical and scientific judgment of the Investigator (i.e., not related to progression of underlying disease), e.g.:
Leading to IMP discontinuation or modification of dosing, or
Fulfilling a seriousness criterion, or
Defined as an AESI.

Exacerbation of a chronic or intermittent pre-existing condition including either an increase in frequency or intensity of the condition.

New conditions detected or diagnosed after study intervention administration even though it may have been present before the start of the study.

Signs, symptoms, or the clinical sequelae of a suspected drug-drug interaction.

Signs, symptoms, or the clinical sequelae of a suspected overdose of either study intervention or a concomitant medication.

“Lack of efficacy” or “failure of expected pharmacological action” per se will not be reported as an AE or SAE. Such instances will be captured in the efficacy assessments. However, the signs, symptoms, or clinical sequelae resulting from lack of efficacy will be reported as AE or SAE if they fulfill the definition of an AE or SAE.

### Example 1.8B1c—Events NOT Meeting the AE Definition

Any clinically significant abnormal laboratory findings or other abnormal safety assessments which are associated with the underlying disease, unless judged by the Investigator to be more severe than expected for the participant's condition.

The disease/disorder being studied or expected progression, signs, or symptoms of the disease/disorder being studied, unless more severe than expected for the participant's condition.

Medical or surgical procedure (e.g., endoscopy, appendectomy): the condition that leads to the procedure is the AE.

Situations in which an untoward medical occurrence did not occur (social or convenience admission to a hospital).

Anticipated day-to-day fluctuations of pre-existing disease(s) or condition(s) present or detected at the start of the study that do not worsen.

### Example 1.8B2—Definition of SAE

An SAE is defined as any adverse event that, at any dose which results in the following.

Results in death.

Is life-threatening: The term “life-threatening” in the definition of “serious” refers to an event in which the participant was at risk of death at the time of the event. It does not refer to an event, which hypothetically might have caused death, if it were more severe.

Requires inpatient hospitalization or prolongation of existing hospitalization: In general, hospitalization signifies that the participant has been admitted (usually involving at least an overnight stay) at the hospital or emergency ward for observation or treatment that would not have been appropriate in the physician's office or outpatient setting. Complications that occur during hospitalization are AEs. If a complication prolongs hospitalization or fulfills any other serious criteria, the event is serious. When in doubt as to whether “hospitalization” occurred or was necessary, the AE should be considered serious. Hospitalization for elective treatment of a pre-existing condition that did not worsen from baseline is not considered an AE.

Results in persistent or significant disability/incapacity.
The term disability means a substantial disruption of a person's ability to conduct normal life functions.
This definition is not intended to include experiences of relatively minor medical significance such as uncomplicated headache, nausea, vomiting, diarrhea, influenza, and accidental trauma (eg, sprained ankle) which may interfere with or prevent everyday life functions but do not constitute a substantial disruption.

Is a congenital anomaly/birth defect.

Other situations.
Medical or scientific judgment should be exercised by the Investigator in deciding whether SAE reporting is appropriate in other situations such as significant medical events that may jeopardize the participant or may require medical or surgical intervention to prevent one of the other outcomes listed in the above definition. These events should usually be considered serious.
Note: The following list of medically important events is intended to serve as a guideline for determining which condition has to be considered as a medically important event. The list is not intended to be exhaustive.
Intensive treatment in an emergency room or at home for:
   Allergic bronchospasm.
   Blood dyscrasias (ie, agranulocytosis, aplastic anemia, bone marrow aplasia, myelodysplasia, pancytopenia, etc).
   Convulsions (seizures, epilepsy, epileptic fit, absence, etc).
Development of drug dependence or drug abuse.
ALT>3×ULN+total bilirubin>2×ULN or asymptomatic ALT increase>10×ULN.
Suicide attempt or any event suggestive of suicidality.
Syncope, loss of consciousness (except if documented as a consequence of blood sampling).
Bullous cutaneous eruptions.
Cancers diagnosed during the study or aggravated during the study.

### Example 1.8B3—Assessment and Follow-Up of AE and/or SAE

### Example 1.8B3a—Assessment of Severity

The Investigator will assess the severity for each AE and SAE using the National Cancer Institute Common Terminology Criteria for Adverse Events (NCI CTCAE) version 5.0, published on 27 Nov. 2017. Listings of MedDRA terms should be consulted first in NCI CTCAE to look for severity grade description for a particular AE. For AEs not listed in the NCI CTCAE, the Investigator will be required to assess the severity of the AE using general guideline:
Grade 1 Mild; asymptomatic or mild symptoms; clinical or diagnostic observations only; intervention not indicated.
Grade 2 Moderate; minimal, local or noninvasive intervention indicated; limiting age-appropriate instrumental ADL*.
Grade 3 Severe or medically significant but not immediately life-threatening; hospitalization or prolongation of hospitalization indicated; disabling; limiting self-care ADL**.
Grade 4 Life-threatening consequences; urgent intervention indicated.
Grade 5 Death related to AE.

### Note: Activities of Daily Living (ADL)

*Instrumental ADL refer to preparing meals, shopping for groceries or clothes, using the telephone, managing money, etc.
**Self-care ADL refer to bathing, dressing and undressing, feeding self, using the toilet, taking medications, and not bedridden.

### Example 1.8B3b—Assessment of Causality

The Investigator is obligated to assess the relationship between study intervention and each occurrence of each AE/SAE.

A “reasonable possibility” of a relationship conveys that there are facts, evidence, or arguments to suggest a causal relationship, rather than that a relationship cannot be ruled out.

The Investigator will use clinical judgment to determine the relationship.

Alternative causes, such as underlying disease(s), concomitant therapy, and other risk factors, as well as the temporal relationship of the event to study intervention administration will be considered and investigated.

The Investigator will also consult the Investigator's Brochure (IB) or Product Information, for marketed products, in his/her assessment.

For each AE/SAE, the Investigator must document in the medical notes that he/she has reviewed the AE/SAE and has provided an assessment of causality.

The Investigator may change his/her opinion of causality in light of follow-up information and send an SAE follow-up report with the updated causality assessment.

The causality assessment is one of the criteria used when determining regulatory reporting requirements.

### Example 1.8B3c—Follow-Up of AEs and SAEs

The Investigator is obligated to perform or arrange for the conduct of supplemental measurements or evaluations as medically indicated to elucidate the nature or causality of the AE or SAE as fully as possible. This may include additional laboratory tests or investigations, histopathological examinations, or consultation with other health care professionals.

### Example 1.8C—Liver and Other Safety: Suggested Actions and Follow-Up Assessments

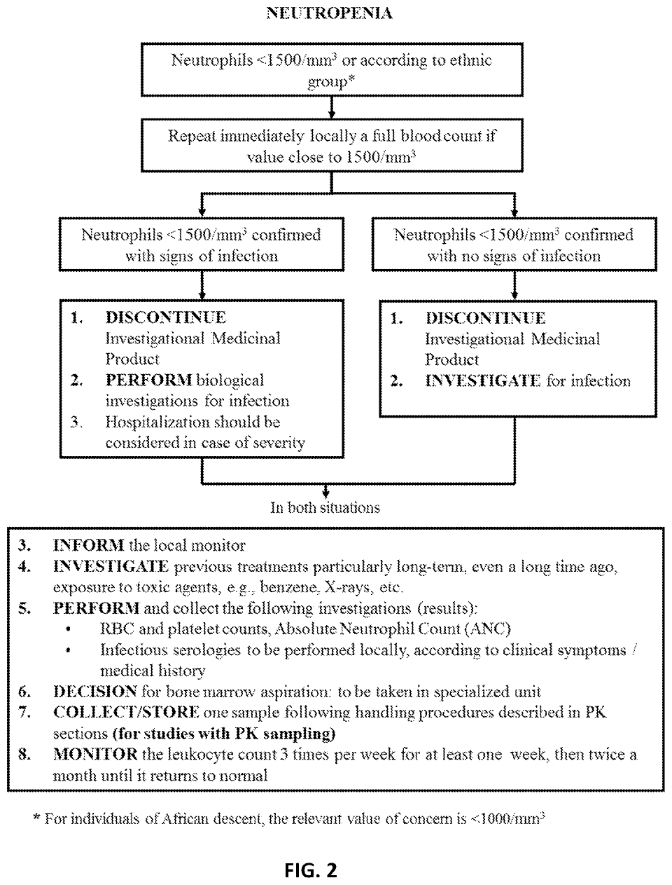

These actions described in Table 10 shown below and FIGS. 2, 3, 4A, 5, and 6 are required for ALT increase and thrombocytopenia events ONLY. For all other safety events described, these are suggested per the Investigator's medical judgement.

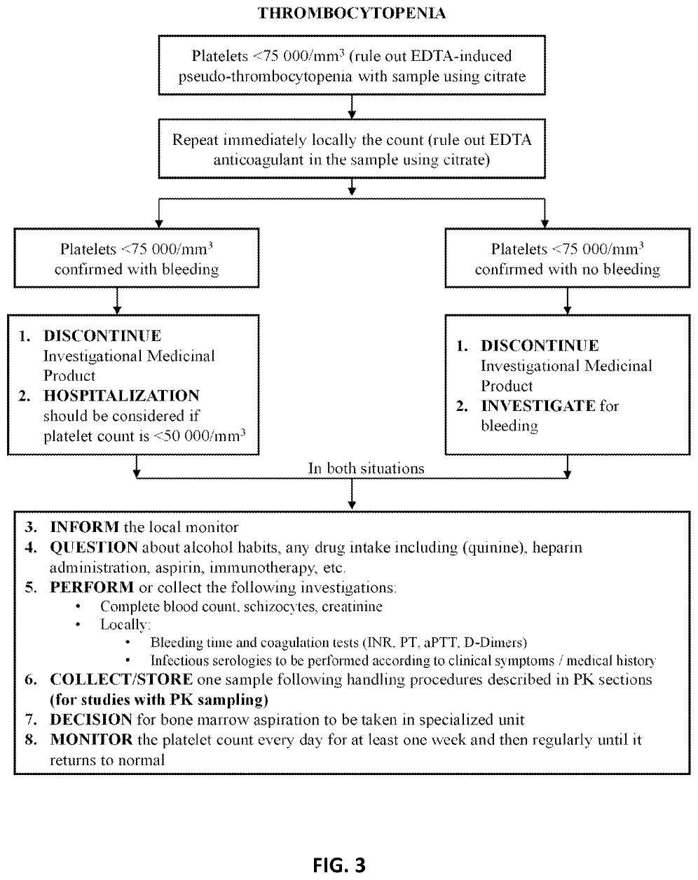

Thrombocytopenia is to be recorded as an AE only if at least 1 of the criteria listed in the general guidelines for reporting adverse events in Example 1.8B is met (FIG. 3).

Neutropenia is to be recorded as an AE only if at least 1 of the criteria listed in the general guidelines for reporting adverse events in Example 1.8B is met.

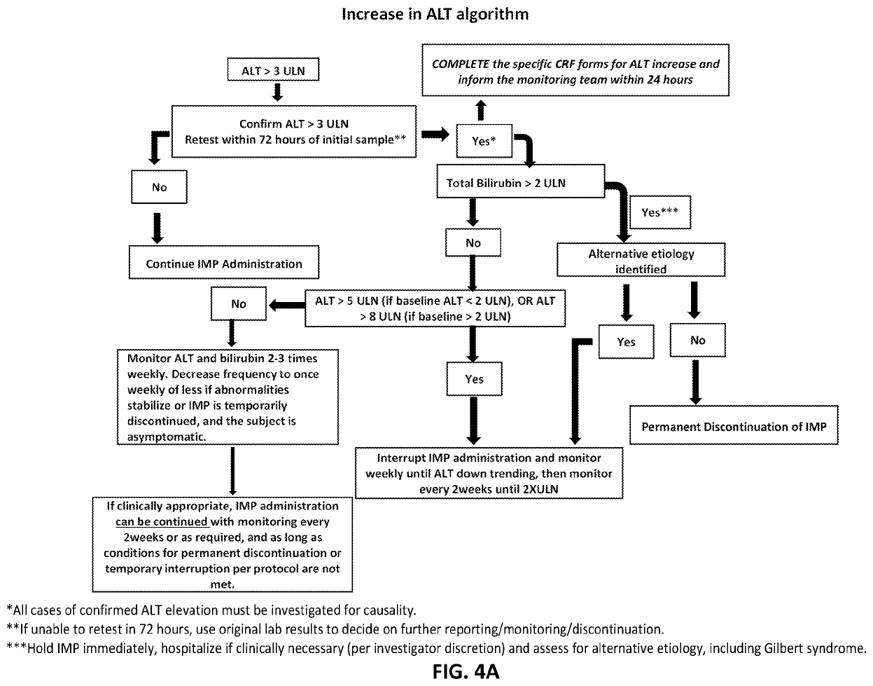

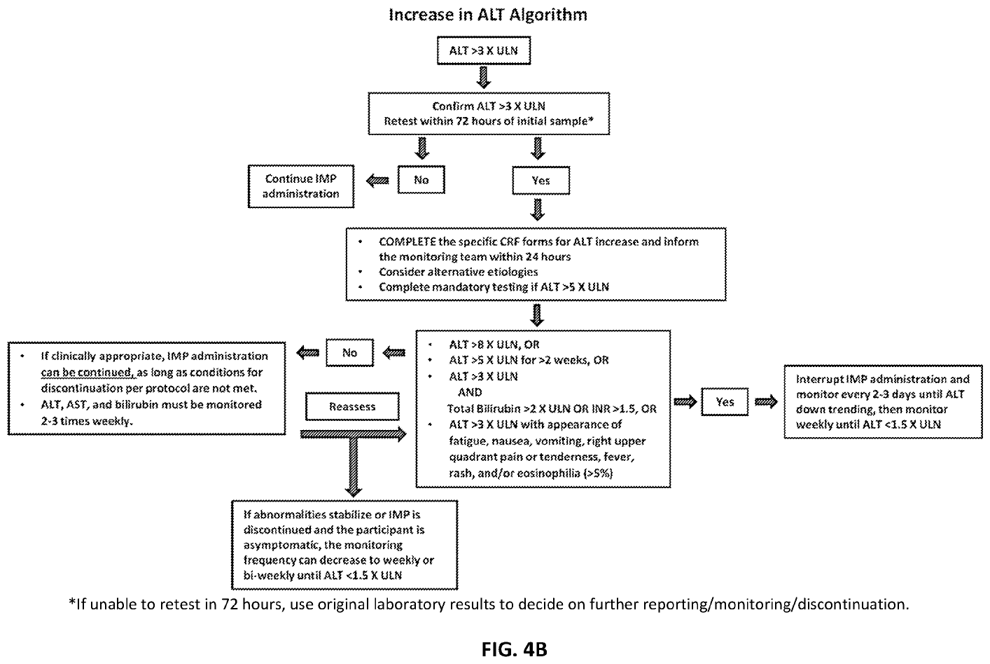

Abbreviations in FIG. 4A: ALT: alanine aminotransferase; AST: aspartate aminotransferase; CMV: cytomegalovirus; CPK: creatine phosphokinase; CRF: case report form; EBV: Epstein-Barr virus; HAV: hepatitis A virus; HBV: hepatitis B virus; HCV: hepatitis C virus; IgM: immunoglobulin M; IMP: investigational medicinal product; INR: international normalized ratio; LFT: liver function test; LKM: liver-kidney microsomal antibody; PT: prothrombin time; ULN: upper limit of normal.

Note: In FIG. 4A, “Baseline” refers to ALT sampled at baseline visit; or if baseline value unavailable, to the latest ALT sampled before the baseline visit. The algorithm does not apply to the instances of increase in ALT during screening.

In FIG. 4A, Normalization is defined as ≤ULN or baseline value, if baseline value is >ULN.

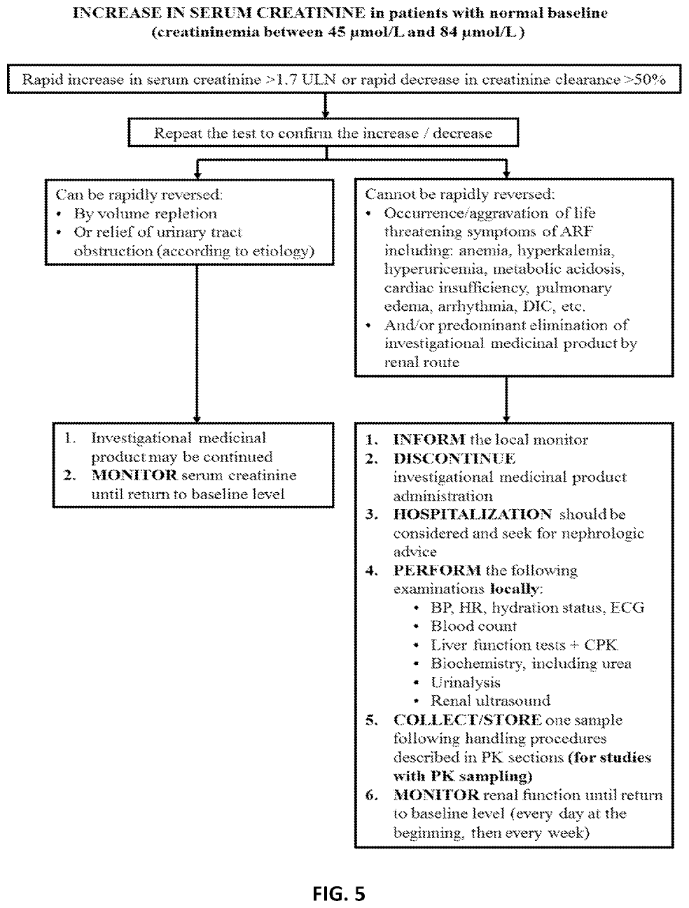

Increase in serum creatinine is to be recorded as an AE only if at least 1 of the criteria listed in the general guidelines for reporting adverse events in Example 1.8B is met (FIG. 5).

Increase in CPK is to be recorded as an AE only if at least 1 of the criteria in the general guidelines for reporting adverse events in Example 1.8B is met.

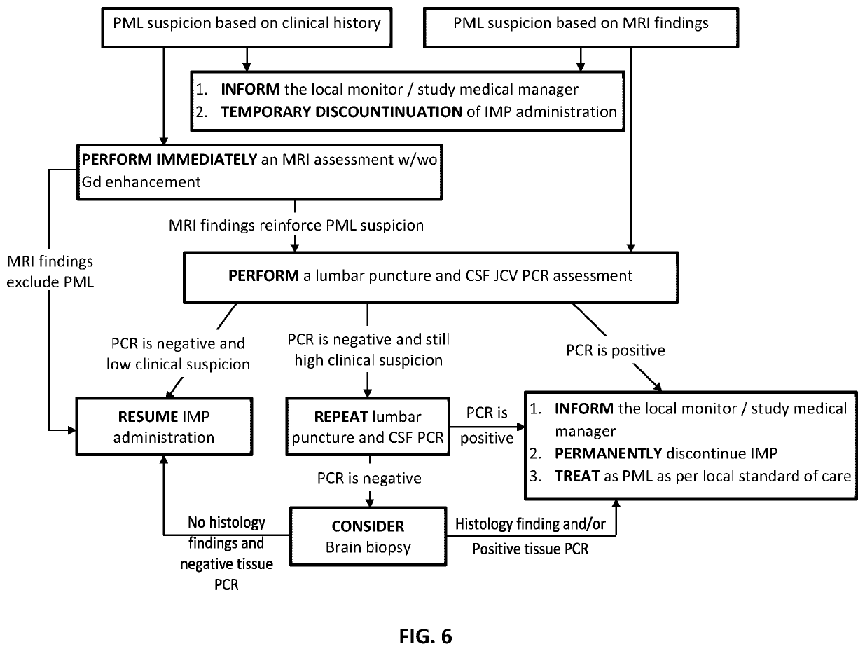

If either the clinical presentation or MRI features of a participant are suggestive of PML, the following diagnostic and action algorithm is recommended (FIG. 6).

Abbreviations in FIG. 6: CSF: cerebrospinal fluid; Gd: gadolinium; IMP: investigational medicinal product; JCV: John Cunningham virus; MRI: magnetic resonance imaging; PCR: polymerase chain reaction; PML: progressive multifocal leukoencephalopathy.

Clinical manifestations or MRI lesions features suspicious for PML are proposed in Table 9.

TABLE 9
Clinical and MRI features suggestive of PML

| | |
|---|---|
| Clinical | Subacute onset of weakness, sensory deficits, cognitive |
| history | or behavioral abnormalities, gait dysfunction, speech/ |
| | language difficulties or any other signs of cortical |
| | dysfunction, retrochiasmal visual defects or seizure |
| Brain MRI | ≥1 T2/FLAIR hyperintense and T1 hypointense lesions |
| | involving the subcortical and juxtacortical white |
| | matter, sparing the cortex, with no mass effect, with a |
| | continuous progression; new lesions with no enhancement |
| | (even when large) or with faint rim enhancement |

In the event that PML is suspected based on imaging results, the local radiologist will directly inform the Investigator and a central review of the MRI will not be required. The Investigator will obtain additional plasma, urine, and CSF samples for John Cunningham virus (JCV) analysis. Samples will be analyzed upon receipt and the results will be provided directly to the investigational site and to the Sponsor. Further management will be deferred to the Treating Investigator. However, next steps will include discontinuation of study treatment. Additional imaging will be at the discretion of the Investigator depending on the diagnostic workup and treatment plan.

The detection of JCV DNA in the CSF of a patient with clinical and MRI features suggestive of PML establishes the diagnosis of PML.

If JCV DNA is not detected in CSF and if clinical suspicion of PML remains high, another lumbar puncture should be performed.

If diagnosis remains uncertain and suspicion of PML remains high, a brain biopsy may be considered to establish a definitive diagnosis.

Clinical or MRI features suggestive of PML should be recorded as an AE/AESI/SAE following the definitions and procedures in Example 1.8B.

TABLE 10
Actions for cases of confirmed ALT elevationIn ANY CONFIRMED
CASE, FOLLOW the instructions listed below:

INFORM the Site Monitor, who will forward the information to the Study Manager.
INVESTIGATE specifically for malaise with or without loss of
consciousness, dizziness, and/or hypotension and/or episode of
arrhythmia in the previous 72 hours; rule out muscular injury
PERFORM the following tests:
LFTs: AST, ALT, alkaline phosphatase, total and conjugated bilirubin
and prothrombin time/INR;
CPK, serum creatinine, complete blood count;
Anti-HAV IgM, anti-HBc IgM, (HBV-DNA if clinically
indicated), anti-HCV and HCV RNA, anti-CMV IgM and anti-HEV IgM antibodies;
Depending on the clinical context, check for recent infection with EBV,
herpes viruses, and toxoplasma;
Consider iron, ferritin, transferrin saturation;
Consider hepatobiliary ultrasonography (or other imaging investigations
if needed);
CONSIDER auto-antibodies: antinuclear, anti-DNA, anti-smooth muscle, anti-
LKM, anti-mitochondrial;
CONSIDER DNA test for Gilbert's disease if clinically indicated;
CONSIDER consulting with a hepatologist;
CONSIDER patient hospitalization if INR >2 (or PT <50%) and/or
central nervous system disturbances suggesting hepatic encephalopathy;
MONITOR LFTs after permanent discontinuation of IMP:
Monitor as closely as possible (every 48 hours to every
week) until ALT is down-trending, then every 2 weeks
until 1.5 × ULN, and then every scheduled visit;
Rechallenge Re-initiation of the study drug can only be considered after
discussion with the Sponsor's Medical Monitor once the ALT/AST
decreases below 2 × ULN, and there is no clinical contraindication. In
case it is agreed to re-start the study drug, it is recommended that
ALT/AST be assessed weekly for the first month and then monthly for
the second and third months. The occurrence of new elevation above 3 ×
ULN for the ALT/AST values will lead to permanent discontinuation
of the study drug.
FREEZE serum sample (5 ml × 2)

### Example 1.8D—Diagnostic Criteria for MOG Antibody Disease

All of the following criteria must be met for the definitive diagnosis of MOG antibody disease (Jarius et al. J Neuroinflammation 2018, 15, 134):
Monophasic or relapsing acute ON, myelitis, brainstem encephalitis, or encephalitis, or any combination of these syndromes.
MRI or electrophysiological (visual evoked potentials in patients with isolated ON) findings compatible with CNS demyelination.
Seropositivity for MOG-IgG as detected by means of a cell-based assay employing full-length human MOG as target antigen.

### Example 1.8E—Abbreviations

ADL: activities of daily living
AE: adverse event
AESI: adverse event of special interest
ALT: alanine aminotransferase
aPTT: activated partial thromboplastin time
ARF: acute renal failure
ARR: annualized relapse rate
BTK: Bruton's tyrosine kinase
CDP: confirmed disability progression
CK-MB: creatine kinase (heart)
CK-MM: creatine kinase (skeletal muscle)
CNS: central nervous system
CPK: creatinine phosphokinase
CRF: case report form
CSF: cerebrospinal fluid
CSR: clinical study report
C-SSRS: Columbia Suicide Severity Rating Scale
CYP: cytochrome P450
DMC: Data Monitoring Committee
DMT: disease-modifying therapies
DNA: deoxyribonucleic acid
DTP: direct to patient
ECG: electrocardiogram, electrocardiography
eCRF: electronic case report form
EDSS: expanded disability status scale
EDTA: Ethylenediaminetetraacetic acid
eGFR: estimated glomerular filtration rate
EOS: end of study
EOT: end of treatment
FSH: follicle stimulating hormone
GCIPL: ganglion cell-inner plexiform layer
GCP: good clinical practice
Gd: gadolinium
HIV: human immunodeficiency virus
HR: hazard ratio
HRT: hormone replacement therapy
IB: Investigators brochure
ICF: informed consent form
ICH: International Council for Harmonisation
IEC: independent ethics committees
Ig: immunoglobulin
IL: interleukin
IMP: investigational medicinal product
INR: international normalized ratio
IRB: institutional review board
ITT: intent-to-treat
IUD: intrauterine device
IUS: intrauterine hormone-releasing system
IV: intravenous
IVIg: intravenous immunoglobulins
JCV: John Cunningham virus
KM: Kaplan-Meier
LFT: liver function tests
LLN: lower limit of normal
MedDRA: Medical Dictionary for Regulatory Activities
MOG: myelin oligodendrocyte glycoprotein
MRI: magnetic resonance imaging
MS: multiple sclerosis
NCI CTCAE: National Cancer Institute Common Terminology Criteria for Adverse Event
NfL: neurofilament light chain
NIMP: noninvestigational medicinal product
NMO: neuromyelitis optica
NSAIDs: nonsteroidal anti-inflammatory drugs
NYHA: New York Heart Association
OCT: optical computerized tomography
ON: optic neuritis
PD: pharmacodynamic
PK: pharmacokinetic(s)
PML: progressive multifocal leukoencephalopathy
PP: per protocol
PT: prothrombin time
QTcF: QT interval corrected using Fridericia's formula
RMS: relapsing multiple sclerosis
RNFL: retinal nerve fiber layer
SAE: serious adverse event
SAP: statistical analysis plan
SoA: schedule of activities
SUSAR: suspected unexpected serious adverse reaction
TB: tuberculosis
TEAE: treatment-emergent adverse event
US: United States
WOCBP: woman of childbearing potential

### Example 2—MOG Antibody Disease (MOGAD) Study of BTK Inhibitor Tolebrutinib

### Example 2.1—Introduction and Study Rationale

Tolebrutinib is a brain penetrant inhibitor of Bruton's tyrosine kinase (BTK). This compound is being developed for the treatment of MOG antibody disease (MOGAD). This study is designed to collect evidence of its efficacy and safety in an active relapsing MOGAG population.

MOGAD is a relapsing, immune mediated disease of the central nervous system that leads to vision loss, weakness, sensory loss, bowel/bladder dysfunction and cognitive dysfunction (Narayan et al. Mult Scler Relat Disord 2018, 25, 66-72). On the spectrum of CNS autoimmune diseases, MOG antibody disease overlaps with multiple sclerosis (MS). Pathologically, anti-MOG appears similar to MS with demyelination and macrophage infiltration with intact axons and limited humoral deposits (Hoftberger et al. Acta Neuropathol 2020, 139, 875-892). Clinically, attacks of MOGAD show a predilection for the optic nerves (66% of attacks) and unlike MS, there is no known progressive course of disease in MOG. That is, if relapses can be prevented, the disease is effectively in remission.

A graphical scheme of the study design is shown in FIG. 1B. Table 11 shown below describes the schedule of activities during the course of study. Table 12 that follows describes the objective and endpoints of the overall study.

TABLE 11
Schedule of Activities (SOA)

| | | | | | | | | | | Follow-up | Treatment | | Follow-up |
|---|---|---|---|---|---|---|---|---|---|---|---|---|---|
| | | Core Study Intervention Period | | | | | | | | Relapse | Extension | End of | Visit |
| | | (every 90 days)⁸ | | | | | | | | Visit | Visit | treatment | (EOS) |
| | | | M 1, | | | | | | Relapse | 4 weeks | Month 18 | (EOT) | 4-6 |
| | Screening | | M 1.5, | | | | | | Evaluation | after | or EOT | Common End⁹ | Weeks |
| | (<28 days | | M 2, | | M 4, | | | | event- | relapse | (if <18 | or Premature | after |
| Procedure | before D 1) | D 1 | M 2.5 | M 3 | M 5 | M 6 | M 9 | M 12 | triggered | onset | months) | Discontinuation | EOT |
| Virtual Visit | X | X | | X | | X | X | X | X | X | X | X | X |
| Informed | X | | | | | | | | | | | | |
| consent | | | | | | | | | | | | | |
| Inclusion and | X | X | | | | | | | | | | | |
| exclusion | | | | | | | | | | | | | |
| criteria | | | | | | | | | | | | | |
| Demography | X | | | | | | | | | | | | |
| Height and | | X | | | | | | | | | | | |
| weight | | | | | | | | | | | | | |
| (patient | | | | | | | | | | | | | |
| reported) | | | | | | | | | | | | | |
| Televideo | X | X | | X | | X | X | X | X | X | X | X | X |
| EDSS | | | | | | | | | | | | | |
| ECG and vital | X | X | | X | | X | X | X | X | X | | X | X |
| signs | | | | | | | | | | | | | |
| (measured | | | | | | | | | | | | | |
| locally)¹ | | | | | | | | | | | | | |
| Medical and | X | | | | | | | | X | X | | | X |
| Neurological | | | | | | | | | | | | | |
| history | | | | | | | | | | | | | |
| Prior / | X | X | X | X | X | X | X | X | X | X | X | X | X |
| concomitant | | | | | | | | | | | | | |
| medications | | | | | | | | | | | | | |
| MOG Ab | X | | | | | X | | X | X | | | X | X |
| titer, IgG and | | | | | | | | | | | | | |
| IgM levels | | | | | | | | | | | | | |
| MRI² | X | | | | | | | X | X | X | | | |
| Screening³: | X | | | | | | | | | | | | |
| pregancy test | | | | | | | | | | | | | |
| (serum), FSH, | | | | | | | | | | | | | |
| coagulation, | | | | | | | | | | | | | |
| tuberculosis | | | | | | | | | | | | | |
| blood testing, | | | | | | | | | | | | | |
| hepatitis B/C | | | | | | | | | | | | | |
| serology, HIV | | | | | | | | | | | | | |
| serology,; | | | | | | | | | | | | | |
| serum anti- | | | | | | | | | | | | | |
| aquaporin 4 | | | | | | | | | | | | | |
| Ab; Iron | | | | | | | | | | | | | |
| panel (serum): | | | | | | | | | | | | | |
| iron, ferritin, | | | | | | | | | | | | | |
| transferrin | | | | | | | | | | | | | |
| saturation, | | | | | | | | | | | | | |
| TIBC; | | | | | | | | | | | | | |
| repeated | | | | | | | | | | | | | |
| during the | | | | | | | | | | | | | |
| study if | | | | | | | | | | | | | |
| needed | | | | | | | | | | | | | |
| Complete | X | X | | X | | X | X | X | X | X | | X | X |
| blood count | | | | | | | | | (if needed) | (if needed) | | | |
| with | | | | | | | | | | | | | |
| differential, | | | | | | | | | | | | | |
| and metabolic | | | | | | | | | | | | | |
| profile⁴ | | | | | | | | | | | | | |
| Liver function | X | X | X | X | X | X | X | X | X | X | X | X | X |
| tests^{x} | | | | | | | | | (if needed) | (if needed) | | | |
| Urinalysis | X | | | X | | | | X | X | X | X | X | |
| | | | | | | | | | (if needed) | (if needed) | | | |
| Urine | | | | X | | X | X | X | | | X | X | X |
| pregnancy test | | | | | | | | | | | | | |
| (done at | | | | | | | | | | | | | |
| home)⁵ | | | | | | | | | | | | | |
| C-SSRS | X | X | | X | | X | X | X | | | X | X | X |
| AE/SAE | X | X | X | X | X | X | X | X | X | X | X | X | X |
| review⁶ | | | | | | | | | | | | | |
| Biomarkers⁷ | | X | | | | X | | X | X | | | X | |
| IMP: | | X | | X | | X | X | X | | | X | | |
| distribution / | | | | | | | | | | | | | |
| reconciliation | | | | | | | | | | | | | |

EOT = End of Treatment;
EOS = End of Study;
FSH = follicle-stimulating hormone;
TIBC = total iron-binding capacity
¹A blood pressure cuff and a remote ECG device that can measure heart rate will be sent to each study participant. These parameters will be measured at all remote visits. In addition participants will be asked to measure blood pressure, heart rate and record an ECG monthly and report the results to the study coordinator each month. In case of any abnormalities in these measurements, the patient may be advised by the Investigator to visit a local hospital for an in person medical evaluation.
²MRI of brain, optic nerves and spinal cord at baseline. MRI for relapse confirmation and follow-up to include relevant regions depending on the clinical presentation and associated neurologic signs and symptoms; optical coherence tomography (OCT) per local practice may also be ordered if needed to confirm relapse.
³Tuberculosis (TB) blood testing is preferred (e.g., QuantiFERON ® TB Gold test); TB test to be repeated based on clinical judgment, borderline results or clinical suspicion of TB infection; HIV serology if allowed per local restrictions; FSH: if needed, only in female participants to confirm postmenopausal state at screening; Lipase, iron, ferritin, transferrin, and calculated transferrin saturation will be tested at the Screening visit only.
⁴Complete blood count with differential, absolute lymphocyte counts (CD19 counts), metabolic profile (blood urea nitrogen [BUN], creatinine, glucose [nonfasting], potassium, sodium, chloride, bicarbonate, calcium,
^{x}Liver function tests: [aspartate aminotransferase (AST), alanine aminotransferase (ALT), albumin, alkaline phosphatase, total and direct bilirubin, total protein; creatine phosphokinase.
⁵Urine pregnancy test: (at home) with result reported to Investigator at the virtual visIt. Pregnancy tests of any type can be requested anytime during the course of the study as determined necessary by the PI or local regulation.
⁶Unscheduled visits may be performed at any time by the Investigator (e.g., for evaluation of an adverse event). Assessments may be done as needed to evaluate the participant in accordance with the Investigator's best judgment and in-line with the study protocol
⁷Biomarkers (blood/serum): neurofilament light chain, glial fibrillary acidic protein level, tau, complement activation marker panel (for both classical and alternative pathway) and B/T/NK cell profiling (potentially in a future analysis). End of study (EOS) biomarker sample is optional for participants who have relapsed during the course of the trial.
⁸A visit window of ±14 days is acceptable for all clinical assessments
⁹Common End: participants may continue treatment beyond month 12 until the last participant reaches Month 12 (or completion of data collection for the primary endpoint).

Objectives and endpoints for the treatment are shown in Table 12.

TABLE 12
Objectives and endpoints

| Objectives | Endpoints |
|---|---|
| Primary | |
| To assess the efficacy of tolebrutinib | Proportion of relapse-free participants at |
| To prevent relapses in participants with | 12 months |
| MOG antibody disease | |
| Secondary | |
| To assess the safety and tolerability of | Adverse events (AEs), serious AEs, AEs |
| tolebrutinib in participants with MOG | leading to permanent study intervention |
| antibody disease | discontinuation, AEs of special interest, |
| | and potentially clinically significant |
| | abnormalities in laboratory tests during |
| | the study period |
| To assess the efficacy of tolebrutinib | Time to first relapse, proportion orelapse- |
| to prevent relapses in participants with | free at 3 months, 6 months, 9 months |
| MOG antibody disease | |
| Exploratory | |
| To assess the effect of tolebrutinib on | Change in T2 lesion burden (number and |
| MRI measures of disease activity | volume) and number of Gd-enhancing |
| | lesions after 1 year of treatment |
| To determine the effect of tolebrutinib | Change from baseline in MOG antibody |
| on MOG antibody titer | titer level after 1 year of treatment |
| To assess the effect of tolebrutinib on | Change from baseline in expanded |
| disability progression | disability status scale score (EDSS) after |
| | 1 year of treatment |
| To explore effects of tolebrutinib on | Changes from baseline in measures of |
| markers of CNS damage | CNS damage including serum (GFAP, , |
| | tau, neurofilament light) after 1 year of |
| | treatment with tolebrutinib among |
| | patients who do not relapse |
| To explore the potential of tolebrutinib | Explore comparison of time to relapse |
| to prevent relapses | during one year of tolebrutinib treatment |
| | versus time to relapse in one year after |
| | initiation of initial MOGAD treatment in |
| | the same participants; or with propensity |
| | score matched external comparison group |
| | Explore time to relapse and annualized |
| | relapse rate over total duration of |
| | tolebrutinib treatment including data |
| | from extension period |
| | Change in CD19 counts in whole blood |
| | after 1 year of treatment |
| To explore other pharmacodynamic (PD) | Change in serum immunoglobulin level |
| effects of tolebrutinib | after 1 year of treatment |

### Appropriateness of Measurements

MOG antibody disease patients appear to accumulate disability in a stepwise progression driven by the relapses that occasionally lead to sustained loss of neurological function (Jurynczyk et al. Brain 2017, 140, 3128-3138). Hence, prevention of relapses is a clinically relevant outcome and is an aim of any potential disease-modifying therapy.

Measurement of neurological disability accumulation is an important clinical endpoint in demyelinating diseases of the central nervous system. The EDSS is widely used to measure neurological disability in clinical trials and routine settings. In this study EDSS will be assessed via a limited neurological exam conducted via tele-video (Bove et al. Mult Scler 2019, 25, 1526-1534). Given the number of participants and duration of this pilot study, change from baseline in EDSS after one year of tolebrutinib treatment will be measured as an exploratory endpoint.

MOG antibody levels over time can be transient in monophasic disease course and persistence is associated with recurrent disease course or poor recovery or in patients who are most likely to experience a relapse (Reindl and Waters Nat Rev Neurol. 2019, 15, 89-102). Persistent MOG Ab titers for more than 6 months are related to a higher risk of a relapsing phenotype that could be present in almost 88% of MOGAD patients (Lopez-Chiriboga et al. JAMA Neurol 2018, 75, 1355-1363). There is also preliminary evidence showing that high MOG antibody titers could be associated with a more aggressive disease (Tea et al. Acta Neuropathol Commun. 2019, 7, 145). Therefore, reducing or eliminating MOG antibody titers may be a positive outcome of the treatment with a potential disease-modifying therapy.

MRI imaging of brain, optic nerve and spinal cord plays an important role in MOGAD diagnosis as well as relapse management with severe attacks accompanied by large T2 lesions in brain or spinal cord. In MOG antibody disease lesions are more likely to resolve completely versus other demyelinating conditions eg (MS and NMO) (Sechi et al. Neurology 2021, 97, e1097-e1109). Optic neuritis is the most common clinical manifestation of MOG antibody disease and therefore optical coherence tomography (OCT) has applicability for confirmation of acute attacks and assessment of damage to the optic nerve (Chen et al. Mult Scler Relat Disorder. 2022, 58, 103525).

BDI-II is a widely used 21-item self-report inventory measuring the severity of depression in adolescents and adults (Beck and Steer 1993 Beck Anxiety Inventory Manual. San Antonio, TX: Psychological Corporation). It can be administered verbally and is suitable for use in virtual visit setting.

In addition to the above-mentioned assessments serum/blood biomarkers measurements will be analyzed in this study as exploratory supportive efficacy data.

### Example 2.2—Study Design

### Example 2.2A—Overall Design

This is a Phase 2, open-label, single arm, single center trial to assess the efficacy and safety of 1-year tolebrutinib 60 mg daily treatment in participants with MOG antibody disease. During screening, participants will discontinue any ongoing immunosuppressive treatment and will receive daily prednisone treatment for 4 weeks during this washout period. After screening, eligible participants will be enrolled to receive 60 mg of tolebrutinib oral daily dose and will complete a steroid taper over the first four weeks of study. The primary endpoint will be the proportion of participants on-treatment and relapse free 1 year after enrollment. Participants in consultation with the investigator may elect to continue study treatment after 1 year of enrollment and remain in the study until the time of last data collection for primary analysis. A common end of study (EOS) will occur for all participants when the last participant enrolled has completed one year of treatment. Therefore, total duration of treatment will vary by individual depending upon recruitment timing, with maximum possible duration of 24 months treatment (e.g. for the first enrolled subject).

The study will be conducted entirely remotely with no in-person visits to the site. All participants will attend virtual visits every 3 months which will include a limited neurological exam conducted via tele-video (Bove et al. Mult Scler 2019, 25, 1526-1534), relapse and adverse event monitoring. Participants will receive sample kits at home and visit remote central laboratory services for collection of samples for MOG antibody titers and other laboratory monitoring. In addition to routine virtual visits, relapse evaluations will be triggered if participants report new neurological symptoms consistent with a MOG antibody disease relapse and include an MRI of the optic nerves and/or spinal cord and/or brain depending on the clinical presentation and associated neurologic signs and symptoms. Optical coherence tomography (OCT) may also be performed if needed to confirm relapse.

Participants who meet criteria for a relapse will be treated for the relapse by their local doctor (according to the local standard of care) and have a remote follow-up relapse visit scheduled for 4 weeks after the onset of the relapse symptoms. Based on individual symptoms and assessed risk of further disease activity, the Investigator and participant may choose for the participant to remain on tolebrutinib until the end of the study.

The Investigator should document the discussion with the participant and the decision for relapse treatment in the source documentation. If there is decision to stop study medication the participant will complete an end of study visit within 4-8 weeks after last dose of study medication.

### Example 2.2B—Number of Participants

Approximately 30 participants will be screened to achieve 25 enrolled.

### Example 2.2C—Intervention Groups and Duration

All participants will be screened over 4 weeks to review the consent form and confirm eligibility for the trial. Participants must complete a washout of any background immunotherapy, and as the duration of washout is dependent upon the half-life of each treatment, this period may need to be initiated prior to the 4 week screening period (see Table 14 exclusion criteria E07). Participants will receive 20 mg daily prednisone through the 4 week screening period while completing washout of any background immunotherapy. Prednisone will then be tapered by 5 mg per day each week by reducing the dose by 5 mg every week at the start of the treatment period until complete discontinuation, concurrent with use of 60 mg daily tolebrutinib.

No additional prednisone or immunotherapies are permitted during the 1-year trial period except as treatment for a confirmed relapse.

### Example 2.2D—Rationale

The aim of this open-label, single-arm phase 2 study is to assess the efficacy and safety of tolebrutinib 60 mg daily in the MOGAD patient population. Efficacy evaluation will primarily focus on assessment of the proportion of patients remaining in remission over one year of treatment. Relapses will be confirmed with objective evidence of lesion on brain or spinal cord MRI corresponding to acute symptoms. This study will provide a preliminary evaluation of evidence of the compound's potential efficacy in the MOGAD population.

### Example 2.2E—Dose Regimen

The choice of the dose of 60 mg tolebrutinib taken with food is based on the results of the Phase 2b dose-finding trial for tolebrutinib in adult participants with relapsing multiple sclerosis (DRI15928).

Analysis of the PK data and effect of fed status on tolebrutinib exposure showed a positive food effect with an increase in AUC0-24 of approximately 2-fold. Moreover, the correlation between the treatment response and the exposure to tolebrutinib showed that higher exposure was associated with low numbers of new gadolinium-enhancing T1-hyperintense lesions after 12 weeks of treatment. Taken together, these data support the recommendation to take tolebrutinib with food.

There was no correlation between the dose of tolebrutinib administered and the number of TEAEs. The most common events (preferred terms) observed in participants in the tolebrutinib treatment arms were headache, upper respiratory tract infection, and nasopharyngitis. There were low numbers of AESIs and PCSAs observed. Overall, no new risks were identified in this trial.

### Example 2.2F—End of Study Definition

A participant is considered to have completed the study by completing all phases of the study including the month 12 visit. However, participants who have a relapse during the course of the trial and discontinue study treatment will be considered to have completed the study if they attend the premature end of treatment visit.

This study will have a common end for all participants after the last data collection for the primary endpoint. The last data collection for primary endpoint occurs when the last participant without a relapse completes the Month 12 visit. With a planned recruitment period of approximately 12 months, a core study treatment period of one year, and follow-up visit 4-8 weeks after last dose the estimated overall duration of the study is approximately 28-32 months.

Duration of study for individuals will vary based on occurrence of relapse and also timing of enrollment. The minimum and maximum duration of treatment for an individual participant that does not experience a relapse will be 12 and 24 months respectively, plus a follow-up visit 4-8 weeks after last dose.

### Example 2.3—Study Population

### Example 2.3A—Inclusion Criteria

Participants are eligible to be included in the study only if all of the following criteria apply as shown in Table 13.

TABLE 13
Inclusion Criteria

| Category | Criteria |
|---|---|
| Age | I 01. The participant must be 18 to 55 years of age inclusive, |
| | at the time of signing the informed consent. |
| Type of | I 02. The participant must have a current diagnosis of MOG |
| participant | antibody disease according to MOG encephalomyelitis: |
| and disease | international recommendations on diagnosis and antibody |
| characteristics | testing (Jarius et al. J Neuroinflammation 2018, 15, 134). |
| | I 03. The participant must have an EDSS score <7.0 at screening. |
| | I 04. The participant must have negative serum aquaporin-4 |
| | antibodies (cell-based assay is the only accepted method). |
| | I 05. The participant must have a documented history of at least 2 |
| | confirmed MOG antibody disease attacks (relapses). |
| | I 06. The participant must have had at least one confirmed MOG |
| | antibody disease relapse in the previous 12 months or 2 |
| | confirmed relapses in the previous 24 months at screening. |
| Weight | I 07. Pediatric participants must have body weight of 40 kg or higher |
| | at study entry. |
| Sex | I 08. Male or Female. |
| | Contraceptive use should be consistent with local regulations |
| | regarding the methods of contraception for those participating in |
| | clinical studies. |
| | c) Male participants |
| | Not applicable. |
| | d) Female participants |
| | A female participant is eligible to participate if she is not |
| | pregnant or breastfeeding, and at least one of the following |
| | conditions applies: |
| | Is not a woman of childbearing potential (WOCBP); or |
| | Is a WOCBP and agrees to use an acceptable contraceptive |
| | method during the intervention period (at a minimum until |
| | after the last dose of study intervention). |
| | A WOCBP must have a negative highly sensitive |
| | pregnancy test (urine or serum as required by local |
| | regulations) (Example 1.8A) at screening before the |
| | first dose of study intervention. If a urine test cannot be |
| | confirmed as negative (eg, an ambiguous result), a serum |
| | pregnancy test is required. In such cases, the participant |
| | must be excluded from participation if the serum |
| | pregnancy result is positive. |
| | Requirements for pregnancy testing during and after study |
| | intervention are located in the schedule of activities (SoA; |
| | Example 1.1). |
| | The Investigator is responsible for review of medical |
| | history, menstrual history, and recent sexual activity to |
| | decrease the risk for inclusion of a woman with an early |
| | undetected pregnancy, if allowed by local regulations. |
| Informed | I 09. Capable of giving signed informed consent which includes |
| Consent | compliance with the requirements and restrictions listed in the |
| | informed consent form (ICF). A specific ICF for legally minor |
| | participants must also be signed by the participant's legally |
| | authorized representative. |

### Example 2.3B—Exclusion Criteria

Participants are excluded from the study if any of the following criteria apply as shown in Table 14.

TABLE 14
Exclusion Criteria

| Category | Criteria | |
|---|---|---|
| Medical | E 01. The participant has a history of infection or may be at risk | |
| conditions | for infection: | |
| | A history of T-lymphocyte or T-lymphocyte-receptor | |
| | vaccination, transplantation (including solid organ, stem | |
| | cell, and bone marrow transplantation) or antirejection | |
| | therapy. | |
| | The participant has received any live (attenuated) vaccine | |
| | (including but not limited to varicella zoster, oral polio, | |
| | and nasal influenza) within 2 months before the first | |
| | treatment visit. | |
| | The participant has a lymphocyte count less than the lower | |
| | limit of normal (LLN) at the Screening Visit. | |
| | A history of diagnosis of progressive multifocal | |
| | leukoencephalopathy (PML) or evidence of findings | |
| | suggestive of PML on the screening MRI. | |
| | A history of infection with human immunodeficiency virus | |
| | (HIV). | |
| | A history of active or latent tuberculosis (TB); TB testing | |
| | should be performed at screening and again during the | |
| | study, if clinically indicated, and may be repeated based | |
| | on clinical judgment, borderline results, or clinical | |
| | suspicion of TB infection. | |
| | NOTE: | |
| | The Investigator may consult with an infectious | |
| | disease expert if required, e.g., test results are unclear or | |
| | there is a suspicion of false positive test results. If the | |
| | infectious disease expert considers the test results as false | |
| | positive and not clinically relevant and confirms that the | |
| | participant can be enrolled in the trial, the Investigator | |
| | must document this in the source data and may then | |
| | randomize the participant. | |
| | Persistent chronic or active or recurring system infection | |
| | that may adversely affect participation or IMP | |
| | administration in this study as judged by the investigator. | |
| | Fever within 4 weeks of the screening visit (≥38° C.; | |
| | however, if due to brief and mild ear, nose, throat viral | |
| | infection participant may be included based on the | |
| | Investigator's judgment). | |
| | Participants at risk of developing or having reactivation of | |
| | hepatitis, i.e., results at screening for serological markers | |
| | for hepatitis B and C indicating acute or chronic infection. | |
| | Serology tests will include hepatitis B virus surface | |
| | antigen, anti-hepatitis B core antigen immunoglobulin M | |
| | (IgM) and total immunoglobulins (Igs), anti-hepatitis B | |
| | surface antigen Igs and anti-hepatitis C virus Igs; in case | |
| | these results are inconclusive (e.g., anti-hepatitis B surface | |
| | antigen negative and anti-hepatitis B core positive or | |
| | anti-hepatitis C virus immunoglobulin G [IgG] positive), | |
| | hepatitis B virus-DNA and/or hepatitis C virus-RNA | |
| | testing, respectively, should be performed for | |
| | confirmation . . . | |
| | E 02. The presence of psychiatric disturbance or substance abuse | |
| | as evidenced by: | |
| | A history of any psychiatric disease, behavioral condition, | |
| | or depression requiring hospitalization within 2 years prior | |
| | to the screening visit. | |
| | A documented history of attempted suicide or suicidal | |
| | ideation or a score of 1 or higher on BDI-2 question on | |
| | suicidal thoughts at baseline/screening version over the 6 | |
| | months prior to the Screening Visit, OR if in the | |
| | Investigator's judgment, the participant is at risk for a | |
| | suicide attempt. | |
| | Active alcohol use disorder or a history of alcohol or drug | |
| | abuse within 1 year prior to the Screening Visit. | |
| | Current alcohol intake >2 drinks per day for men and >1 | |
| | drink per day for women (1 drink = approximately 14 | |
| | grams of alcohol = 350 mL beer = 140 mL wine = 40 mL | |
| | of spirits) | |
| | E 03. Any confirmed abnormal liver function test at screening | |
| | (e.g., aspartate aminotransferase (AST), alanine | |
| | aminotransferase (ALT), albumin, alkaline phosphatase, | |
| | total and direct bilirubin, total protein) | |
| | E 04. The following findings obtained during the Screening Visit | |
| | considered in the Investigator's judgment to be clinically | |
| | significant in the context of this trial: | |
| | Any screening laboratory values outside normal limits. | |
| | Abnormal ECG. | |
| | E 05. Any conditions that may predispose the participant to | |
| | excessive bleeding: | |
| | A bleeding disorder or known platelet dysfunction at any | |
| | time prior to the screening visit. | |
| | A platelet count <150 000/μL at the screening visit. | |
| | The participant has had major surgery within 4 weeks prior | |
| | to the screening visit, which could affect the participant's | |
| | safety (as judged by the Investigator) or has planned any | |
| | elective major surgery during the study. | |
| | A history of significant bleeding event within 6 months | |
| | prior to screening, according to the Investigator's judgment | |
| | such as, but not limited to cerebral or gastrointestinal | |
| | bleeding. | |
| | E 06. Conditions that would adversely affect participation in the | |
| | study or make the primary efficacy endpoint non- | |
| | evaluable: | |
| | A short life expectancy due to pre-existing health | |
| | condition(s) as determined by their treating neurologist. | |
| | A history or presence of significant other concomitant | |
| | illness according to the Investigator's judgment such as, | |
| | but not limited to cardiovascular (including Stage III or IV | |
| | cardiac failure according to New York Heart Association | |
| | [NYHA] classification), or renal (i.e., undergoing dialysis), | |
| | neurological, endocrine, gastrointestinal, hepatic | |
| | (i.e., underlying hepatobiliary disease, metabolic, | |
| | pulmonary, or lymphatic disease that would adversely | |
| | affect participation in this study. | |
| | Acute liver disease, cirrhosis, chronic liver disease (unless | |
| | considered stable for >6 months) | |
| | Confirmed screening ALT >1.5 × ULN OR AST >1.5 × | |
| | ULN OR alkaline phosphatase >2 × ULN (unless caused | |
| | by non-liver-related disorder or explained by a stable | |
| | chronic liver disorder) OR total bilirubin >1.5 × ULN | |
| | (unless due to Gilbert syndrome or non-liver-related | |
| | disorder) | |
| | At screening, elevated transferrin saturation (>50% in | |
| | males and >40% in females) and/or with elevated ferritin | |
| | levels >500 μg/L | |
| | Any malignancy within 5 years prior to the Screening Visit | |
| | (except for effectively treated carcinoma in situ of the | |
| | cervix or adequately treated non-metastatic squamous or | |
| | basal cell carcinoma of the skin). | |
| | Any other medical condition(s) or concomitant disease(s) | |
| | making them non-evaluable for the primary efficacy | |
| | endpoint or that would adversely affect participation in this | |
| | study as judged by the Investigator. | |
| Prior/concomitant | E 07. Participant has received or requires concomitant treatment | |
| therapy | with any of the following medications/treatments within | |
| | the specified time frame before any baseline (Day 1) | |
| | assessment: | |
| | Medication | Exclusionary if |
| | | used/used within |
| | | required wash- |
| | | out period |
| | High dose systemic corticosteroids | 1 month prior to baseline |
| | (pulse therapy) | |
| | Plasma exchange | 1 month prior to baseline |
| | IV immunoglobulin | 2 months prior to baseline |
| | Mildly to moderately | 3 months prior to baseline |
| | immunosuppressive/chemotherapeutic | |
| | medications such as azathioprine and | |
| | methotrexate, mycophenolate | |

| | |
|---|---|
| | E 09. The participant is receiving potent and moderate inducers |
| | of cytochrome P450 3A (CYP3A) or potent inhibitors of |
| | CYP2C8 hepatic enzymes. |
| | E 10. The participant is receiving anticoagulant/antiplatelet |
| | therapies, including: |
| | Acetylsalicylic acid (aspirin) >81 mg/day, |
| | Antiplatelet drugs (eg, clopidogrel), |
| | Warfarin (vitamin K antagonist), |
| | Heparin, including low molecular weight heparin |
| | (antithrombin agents), |
| | Dabigatran (direct thrombin inhibitor), |
| | Apixaban, edoxaban, rivaroxaban (direct factor Xa |
| | inhibitors). |
| | Note: |
| | All the above-mentioned drugs must be stopped at least 5 |
| | half-lives before study drug administration except for aspirin, |
| | which must be stopped at least 8 days before. If the participant has |
| | a chronic underlying medical condition (stroke, coronary or |
| | carotid artery disease, heart valvular disease etc.) requiring |
| | continued use of these medications, the participant cannot be |
| | enrolled in the study. |
| | E 11. The participant has sensitivity to any of the study |
| | interventions, or components thereof, or has a drug or other |
| | allergy that, in the opinion of the Investigator, |
| | contraindicates participation in the study. |
| Prior/concurrent | E 11. The participant was previously exposed to any BTK |
| clinical study | inhibitor, including tolebrutinib. |
| experience | E 12. The participant has taken other investigational drugs within |
| | 3 months or 5 half-lives, whichever is longer, before the |
| | screening visit. |
| Diagnostic | E 13. The participant has a contraindication for MRI, ie, presence |
| assessments | of pacemaker, metallic implants in high-risk areas (ie, |
| | artificial heart valves, aneurysm/vessel clips), presence of |
| | metallic material (eg, shrapnel) in high risk areas, known |
| | history of allergy to any contrast medium, or history of |
| | claustrophobia that would prevent completion of all |
| | protocol scheduled MRI. |
| | Note: |
| | People with a contraindication to Gd can be enrolled into |
| | the study but cannot receive Gd contrast dyes during their MRI |
| | scan. |
| Other exclusions | E 14. Individuals accommodated in an institution because of |
| | regulatory or legal order; prisoners or participants who are |
| | legally institutionalized |
| | E 15. Participant not suitable for participation, whatever the |
| | reason, as judged by the Investigator, including medical or |
| | clinical conditions, or participants potentially at risk of |
| | noncompliance to study procedures |
| | E 16. Participants are employees of the clinical study site or |
| | other individuals directly involved in the conduct of the |
| | study, or immediate family members of such |
| | individuals (in conjunction with section 1.61 of the ICH- |
| | GCP Ordinance E6) |
| | E 17. Any specific situation during study implementation/course |
| | that may raise ethical considerations |
| | E 18. Sensitivity to any of the study interventions, or components |
| | thereof, or drug or other allergy that, in the opinion of the |
| | Investigator, contraindicates participation in the study |
| | Note: |
| | a one-time retest at screening may be performed if an |
| | abnormal laboratory test value is considered temporary |
| | Prospective approval of protocol deviations to recruitment and |
| | enrollment criteria, also known as protocol waivers or exemptions, |
| | is not permitted |

### Example 2.3C—Lifestyle Considerations

Meals and dietary restrictions: tolebrutinib (IMP) shall be taken with a regular meal. When possible, the meal with which IMP is taken (e.g., breakfast, lunch, or dinner) should be consistent throughout the study. The typical meal with which IMP is taken will be collected at each visit. In case the mealtime needs to be changed for IMP administration, a gap of a minimum of 12 hours between 2 doses should be maintained.

Caffeine, alcohol, and tobacco: During the entire study, the IMP should be used with caution in participants who should be warned not to consume substantial quantities of alcohol, defined as >20 gr (2 glasses 14 grams (1 standard drink) per day in female participants or >30 gr (3 glasses 28 grams (2 standard drinks) per day in male participants on a regular basis; or >40 gr (4 glasses) per day on occasion.

### Example 2.4—Study Intervention(s) and Concomitant Therapy

Study intervention is defined as any investigational intervention(s), marketed product(s), placebo, or medical device(s) intended to be administered to a study participant according to the study protocol.

### Example 2.4A—Study Intervention(s) and Concomitant Therapy Administered

This study intervention includes an IMP and a noninvestigational medicinal product (NIMP). To maintain blinding, participants receive 4 tablets once per day of the BTK inhibitor or placebo in a blinded fashion. Details for the interventions are provided in Table 15.

TABLE 15
Overview of study interventions administered

| | |
|---|---|
| Intervention label | tolebrutinib |
| Intervention name | tolebrutinib 60 mg |
| Type | Drug |
| Dose formulation | Film coated tablet |
| Unit dose strength(s) | 60 mg |
| Dosage level(s) | Once daily |
| Route of administration | Oral |
| Use | Experimental |
| IMP or NIMP | IMP |
| Packaging and labeling | Study Intervention will be provided in wallet blister |
| | packing. Each package will be labeled as required per |
| | country requirement. |
| Current name | tolebrutinib |

*IMP: investigational medicinal product;
NIMP: noninvestigational medicinal product

Details of noninvestigational medicinal products are provided in Table 16.

TABLE 16
Overview of Noninvestigational Medicinal Products

| | |
|---|---|
| MRI contrast- | Route(s) of administration: IV. |
| enhancing preparations | Dose regimen: as per respective label. |
| (only in the case of a | |
| suspected relapse) | |
| Prednisone | Route(s) of administration: Oral. |
| | Dose regimen: 20 mg daily prednisone for 4 |
| | weeks during the screening period, followed |
| | by weekly weaning by 5 mg daily for an |
| | additional 4 weeks at the start of the |
| | treatment period. |
| Relapse treatment | Option #1 |
| recommendations: | Formulation: Methylprednisone 1000 mg. |
| | Route(s) of administration: Intravenous. |
| | Dose regimen: Daily methylprednisone for 3-7 |
| | consecutive days upon confirmation of |
| | inflammatory MOG antibody disease relapse. |
| | Oral prednisone taper of 8 weeks follows |
| | completion of intravenous therapy starting at |
| | 60 mg daily with decreases in the daily dose |
| | by 10 mg every week. |
| | Option #2 |
| | Formulation: 10% Human purified |
| | immunoglobulin 2 grams/kg of ideal body weight. |
| | Route(s) of administration: Intravenous. |
| | Dose regimen: Up to 5 daily infusions of 0.4 |
| | grams/kg upon confirmation of inflammatory |
| | MOG antibody disease relapse. |
| | No prednisone taper required or recommended. |

### Example 2.4B—Measures to Minimize Bias: Randomization and Blinding

This is an open-label study; potential bias will be reduced by the following steps: Objective measures will be used to determine the primary outcome of relapse. Relapse is defined clinically, but also require MRI confirmation of the attack that explains the clinical symptoms.

### Example 2.4C—Dose Modification

Dose modification is not foreseen in this study. Treatment may need to be interrupted or permanently discontinued if deemed necessary due to an AE or relapse.

### Example 2.4D—Continued Access to Intervention after the End of the Study

No study intervention with tolebrutinib is currently planned after the end of the study.

### Example 2.4E—Concomitant Therapy

Any medication or vaccine (including over-the-counter or prescription medicines, vitamins, or herbal supplements) that the participant is receiving at the time of enrollment or receives during the study must be recorded along with: Reason for use; Dates of administration including start and end dates; Dosage information including dose and frequency.

Live (attenuated) vaccines should not be administered during the intervention period.

Therapies for MOG antibody disease noted in the exclusion criterion E07 are not permitted after enrollment and during the core study intervention period. Short-term use (3 to 7 days) of glucocorticoids (e.g., for an acute illness) and local corticosteroids (e.g., topical, nasal, ocular, otic, intra-articular) are allowed. In case of relapse, acute treatment may be considered such as recommended in Table 16, relapse treatment recommendations. Furthermore, the participant in consultation with the investigator and treating neurologist should evaluate the benefit and risk of continuing study treatment.

Participants must abstain from taking prescription or nonprescription drugs (including vitamins and dietary or herbal supplements) within 7 days (or 14 days if the drug is a potential enzyme inducer) or 5 half-lives (whichever is longer) before the start of study intervention until completion of the follow-up visit, unless, in the opinion of the Investigator and Sponsor, the medication will not interfere with the study.

Medications for the treatment of MOG antibody disease symptoms (e.g., walking impairment, spasticity, incontinence, pain) should be maintained at a stable dose prior to screening and for the duration of the treatment period, if clinically feasible.

Anticoagulant/antiplatelet therapies are not permitted to be taken concomitantly with the IMP, including: Acetylsalicylic acid (aspirin)>81 mg/day; Antiplatelet drugs (eg, clopidogrel); Warfarin (vitamin K antagonist); Heparin, including low molecular weight heparin (antithrombin agents); Dabigatran (direct thrombin inhibitor); Apixaban, edoxaban, rivaroxaban (direct factor Xa inhibitors).

Paracetamol/acetaminophen, at doses of ≤3 grams/day, is permitted for use at any time during the study. Short courses (up to 5 days) of nonsteroidal anti-inflammatory drugs (NSAIDs) (other than acetylsalicylic acid), preferably selective cyclooxygenase-2 inhibitors such as NSAIDs at the lowest effective dose, may be given during the study if clinically necessary for the treatment of an existing medical condition or a new event. The Investigator must record the use of NSAIDs (and any other comedication) in the eCRF.

CYP Inhibitor/Inducer: Potent and moderate inducers of CYP3A or potent inhibitors of CYP2C8 hepatic enzymes are not allowed throughout the study.

### Example 2.5—Discontinuation of Study Intervention and Participant Discontinuation/Withdrawal

### Example 2.5A—Discontinuation of Study Intervention

### Example 2.5A1—Permanent Discontinuation

The study intervention should be continued whenever possible, except potentially in case of MOG antibody disease relapse. In case of relapse, the participant in consultation with the investigator and treating neurologist should evaluate the benefit and risk of continuing study treatment or may choose to switch to another immunomodulatory/immunosuppressive therapy. Permanent intervention discontinuation is any treatment discontinuation associated with the definitive decision by the Investigator or the participant not to re-expose the participant to study intervention at any time.

The following may be justifiable reasons for the Investigator to discontinue a participant from study treatment:
Adverse events that endanger the safety of the participant, or if discontinuation of study intervention is desired or considered necessary by the Investigator and/or participant.
If IMP discontinuation criteria are met as per the guidance for the follow up of laboratory abnormalities (Example 2.8A).
The participant is no longer deriving a therapeutic/clinical benefit in the opinion of the Investigator.
At participant's request, i.e., withdrawal of the consent for treatment.
If a female participant becomes pregnant or wishes to become pregnant during the study.
Any serious opportunistic infections.
Continued need for chronic use of a prohibited concomitant medication.

Discontinuation of study intervention for abnormal liver function should be considered by the Investigator when a participant meets one of the conditions outlined in the algorithm (Example 2.8C) or if the Investigator believes that it is in the best interest of the participant.

Any clinically significant abnormal laboratory value or ECG parameter will be immediately rechecked for confirmation after 24 hours before making a decision of definitive discontinuation of the IMP for the concerned participant.

If a clinically significant finding is identified in the ECG (including, but not limited to changes from baseline in QT interval corrected using Fridericia's formula [QTcF]) after enrollment, the Investigator or qualified designee will determine if the participant can continue in the study and if any change in participant management is needed. Review of ECG findings by a cardiologist may be considered for a decision of a definitive discontinuation of study intervention because of ECG changes. This review of the ECG printed at the time of collection must be documented. Any new clinically relevant finding should be reported as an AE. See the SoA (Example 2.1) for data to be collected at the time of intervention discontinuation and follow up and for any further evaluations that need to be completed.

If study intervention is permanently discontinued, the participant shall be asked to complete a premature end of treatment visit. See the SoA (Example 2.1) for data to be collected at the time of the visit and follow up and for any further evaluations that need to be completed. All cases of definitive study drug discontinuation must be recorded in the appropriate pages of the eCRF when considered as confirmed.

### Example 2.5A2—Temporary Discontinuation

Temporary intervention discontinuation because of suspected AEs or disruption of the clinical trial due to a regional or national emergency declared by a governmental agency: Contingency measures for a regional or national emergency that is declared by a governmental agency) may be considered by the Treating Investigator. For all temporary intervention discontinuations, duration should be recorded by the Investigator in the appropriate pages of the eCRF.

If surgery is needed during the study, consider the benefit-risk of withholding the IMP for at least 3 to 7 days pre- and post-surgery depending upon the type of surgery and the risk of bleeding.

The following shall lead to temporary treatment discontinuation:
Cytopenia: follow Sanofi algorithm for neutropenia and thrombocytopenia as per Example 2.8C.
Serum creatinine, creatine phosphokinase (CPK) and liver enzyme increase: follow corresponding Sanofi algorithms as per Example 2.8C.
Cardiac arrhythmia (atrial fibrillation): Any Grade 3 event (symptomatic, urgent intervention indicated; device [e.g., pacemaker]; ablation; new onset).
Suicidal risk as per BDI: if a participant scores 1 or higher.

If needed, temporary treatment discontinuation can be considered by the Investigator or by the participant for any other reason, including due to any safety concerns because of disruption of the clinical trial due to a regional or national emergency declared by a governmental agency such as COVID-19 or another illness or if there is a need for a prohibited concomitant medication. Treatment can be resumed later when it is considered safe and appropriate, and with consideration of remaining duration of the overall study.

### Example 2.5A3—Rechallenge

Re-initiation of the IMP will be done under close and appropriate clinical/and or laboratory monitoring once the Investigator will have considered according to his/her best medical judgment that the responsibility of the IMP(s) in the occurrence of the concerned event was unlikely, there are no safety concerns and if the criteria for permanent treatment discontinuation have not been met.

### Example 2.6—Study Assessments and Procedures

Study procedures and their timing are summarized in the SoA (Example 2.1). Protocol waivers or exemptions are not allowed.

Adherence to the study design requirements, including those specified in the SoA (Example 2.1), is essential and required for study conduct.

All screening evaluations must be completed and reviewed to confirm that potential participants meet all eligibility criteria. The Investigator will maintain a screening log to record details of all participants screened and to confirm eligibility or record reasons for screening failure, as applicable.

Procedures conducted as part of the participant's routine clinical management (e.g., blood count) and obtained before signing of the ICF may be utilized for screening or baseline purposes provided the procedures met the protocol-specified criteria and were performed within the time frame defined in the SoA (Example 2.1). Such assessments shall be recorded on the designated field in the CRF.

Blood sampling details including volume for all laboratory assessments will be provided in the informed consent form. Repeat or unscheduled samples may be taken for safety reasons or for technical issues with the samples.

### Example 2.6A—Efficacy Assessments

Planned time points for all efficacy assessments are provided in the SoA (Example 2.1).

Key efficacy assessments (eg, EDSS) will be scored by the study team. Expanded Disability Status Scale is a method of quantifying disability and monitoring changes in the level of disability over time (Kurtzke Neurology 1983, 33, 1444-1452). It is widely used in clinical trials and in the assessment of people with MS, NMO and MOGAD. The EDSS scale ranges from 0 to 10 in 0.5 unit increments that represent higher levels of disability. Scoring is based on an examination by a neurologist and weighs heavily towards mobility (spinal cord) and vision (optic nerves). EDSS will be conducted using a remote telephone-video enabled examination (Bove et al. Mult Scler 2019, 25, 1526-1534).

The Investigator will be responsible for the confirmation of relapses. MRI findings and local neurologist assessment during the acute attack will be needed in these cases. The MRI scans for relapse confirmation and follow-up relapse visit will be analyzed by the Investigator.

The basic MRI scan will be performed locally and will consist of the following sequences: T2- and T1-weighted sequences before and after administering a Gd contrast (if there is no contraindication). MRI scan shall include optic nerves and/or spinal cord and brain at baseline and MRI for relapse should include relevant regions depending on the clinical presentation and associated neurologic signs and symptoms.

The Investigator may request the participant to undergo optical coherence tomography (OCT) assessment OCT to verify relapse, if needed.

MOG antibody testing performed by cell based assay includes both a positive/negative result, as well as a titer. The titer is the reported as the highest dilution in which the MOG antibody is still detectable. Dilutions at Quest include 1:10, 1:20, 1:40, 1:80, 1:160 and so on. The higher the titer, the greater the amount of MOG antibody in the serum.

### Example 2.6A1—Definition of MOG Antibody Disease Relapse

For the purposes of this study, MOG antibody disease relapse is defined as a monophasic, acute or subacute onset of, new neurological symptoms or worsening of previous neurological symptoms with an objective change on neurological examination. Symptoms must:
Be attributable to MOG antibody disease,
Last for ≥24 hours, with or without recovery,
Be present at normal body temperature (ie, no infection, excessive exercise, or excessively high ambient temperature),
Be preceded by ≥30 days of clinical stability (including no previous relapse), and
Be attributable to a new or enhancing MRI lesion(s) in the brain or the spinal cord and/or the optic nerves.
   Note: An exacerbation or recurrence of symptoms and signs in a participant with MOG antibody disease that can be reasonably attributed to transient impairment of conduction in previously demyelinated pathways due to raised core body temperature will not be considered a relapse.

Confirmation of MOG antibody disease relapse will be done by the Investigator based on the following definition:
A confirmed MOG antibody disease relapse is one accompanied by a clinically relevant change in the EDSS score performed by the local neurologist, i.e., an increase of at least 0.5 points in the EDSS score, an increase of 1 point on 2 functional scores, or an increase of 2 points on 1 functional score, excluding changes involving bowel/bladder and cerebral functional score compared to the previously available rating (the last EDSS rating that did not occur during a relapse).
MRI (brain and/or spinal cord and/or optic nerves) must be performed as soon as possible to support the confirmation of a relapse.

### Example 2.6A2—Relapse Evaluation and Follow-Up Relapse Visits

Participants must be instructed to immediately report new neurological symptoms and recurring or worsening of previous symptoms to the Investigator. In addition, the participant will be actively asked about possible relapse symptoms at each study visit.

If relapse is suspected, a relapse evaluation visit with the Investigator must be scheduled as soon as possible (whenever possible within 7 days of onset of the symptoms). The assessment and confirmation of MOG antibody disease relapse is made by the Investigator. The management of the relapse will be done locally by the treating neurologist according to local standard of care. Recommendations for the treatment of relapses are detailed in Table 16 and are not mandatory.

Diagnosing MOG antibody disease relapses during the study: Participants are instructed to immediately notify the study team of any new visual symptoms, weakness, numbness or bowel/bladder dysfunction. Relapse assessment will be triggered when participants report new or worsening neurological symptoms to the study team, ideally within 72 hours of onset of change/new symptoms. At this point, a virtual EDSS exam will be performed remotely. The PI will determine if the constellation of signs and symptoms should prompt a relapse evaluation by MRI of the affected area. An on-trial relapse is confirmed in this clinical context by a new T2 or enhancing lesion by MRI that corresponds to the signs and symptoms (see Example 2.6A1).

Whether the event is adjudicated as a relapse or not, the PI will not interfere with treatment using steroids or IVIG/PLEX if the local physician/neurologist insists on treatment. The final determination of the relapse is the responsibility of the PI, but the final decision to treat for a relapse or not will be determined by the local physician/neurologist.

A negative MRI or other clinical clues of pseudorelapse will not count as an adjudicated relapse for the purposes of this study. A pseudorelapse will be managed by the local physician or neurologist as routine clinical care.

All confirmed MOG antibody disease relapses must be reported on the relapse eCRF page. MOG antibody disease relapse should not be reported as an AE unless, in the judgment of the Investigator, it is unusually severe or medically unexpected, or matches definition of an SAE. The exception to this definition of an SAE is in the event that a participant is hospitalized to receive standard treatment for relapse, some sites routinely hospitalize participants who require administration of intravenous medication to treat a relapse. Thus, the SAE criteria for “hospitalization” would be met on the basis of local practice and would not reflect the seriousness of the event. When the relapse results in hospitalization for any reason other than for routine treatment of the relapse (such as for a treatment course beyond the standard treatment described in Table 16 or when hospitalization is prolonged, the MOG antibody disease relapse should be considered a serious adverse event.

Safety laboratory tests are optional for this relapse evaluation visit if no intercurrent disease is suspected. If any intercurrent disease is diagnosed, it will be reported as an AE as per the safety reporting rules.

A follow-up relapse visit will be performed by the Investigator 4 weeks after a confirmed relapse as per the SoA (Example 2.1).

### Example 2.6B—Safety Assessments

Planned time points for all safety assessments are provided in the SoA (Example 2.1).

### Example 2.6B1—Electrocardiograms

ECG will be obtained as outlined in the SoA (Example 1.1) using a smart-phone enabled ECG home device that automatically calculates the heart rate and measures PR, QRS, QT, and QTcF intervals. In case the ECG device does not automatically calculate QTcF, manual calculation using nomogram or automatic website calculator (eg, https.//reference.medscape.com/calculator/48/ecg-corrected-qt) is acceptable.

### Example 2.6B2—Vital Signs

Body temperature, heart rate, and blood pressure will be assessed remotely at home by the participant using a smart-phone enabled home device.

Blood pressure and heart rate measurements will be assessed with the participant in a supine or sitting position with a completely automated device. Manual techniques will be used only if an automated device is not available.

Blood pressure and heart rate measurements should be preceded by at least 5 minutes of rest for the participant in a quiet setting without distractions (eg, television, cell phones).

Heart rate and blood pressure measurements will be taken before blood collection for laboratory tests and will consist of 1 heart rate and 3 blood pressure measurements (3 consecutive blood pressure readings will be recorded at intervals of at least 1 minute).

### Example 2.6B3—Clinical Safety Laboratory Assessments

See Example 2.8A for the list of clinical laboratory tests to be performed. Per the SoA (Example 2.1), serology tests for hepatitis B and C will be performed during screening.

The Investigator may solicit emergency local laboratory data in case of emergent safety events to allow for appropriate treatment decisions. All clinically relevant solicited emergency local laboratory data will be recorded in the eCRF.

The Investigator must review the laboratory report, document this review, and record any clinically relevant changes occurring during the study in the AE section of the eCRF. The laboratory reports must be filed with the source documents. Clinically significant abnormal laboratory findings are those which are not associated with the underlying disease, unless judged by the Investigator to be more severe than expected for the participant's condition.

All laboratory tests with values considered clinically significantly abnormal during participation in the study or within 28 days after the last dose of study intervention should be repeated until the values return to normal or baseline or are no longer considered clinically significant by the Investigator.

If abnormal laboratory test values do not return to normal or baseline within a period of time judged reasonable by the Investigator, the etiology should be identified, and the Sponsor notified.

All protocol-required laboratory assessments, as defined in Example 2.8A must be conducted in accordance with the SoA. In the event the laboratory assessments in Example 2.8C indicate discontinuation of IMP, temporary interruption should be considered unless otherwise specified.

If laboratory values from non-protocol-specified laboratory assessments performed at the institution's local laboratory require a change in participant management or are considered clinically significant by the Investigator (eg, SAE or AE or dose modification), then the results must be recorded in the eCRF.

### Example 2.6C—Adverse Events (AEs), Serious Adverse Events (SAES) and Other Safety Reporting

### Example 2.6C1—MOG Antibody Disease Relapse Reporting

MOG antibody disease relapses, determined from the evaluations described in 2.6A1, will be exempt from being reported as AEs except when they meet the definition of a SAE, or are unusually severe or medically unexpected. Hospitalization for a relapse, if done routinely at the site (eg, for high dose IV methylprednisolone), will not be considered as a seriousness criterion for this study.

Data for MOG antibody disease relapses will be collected on the eCRF and be analyzed as part of the efficacy analysis. Other worsening of neurological symptoms that do not meet the definition of MOG antibody disease relapse will be reported as AEs according to general safety reporting rules.

### Example 2.6C2—Reporting of Safety Findings from Magnetic Resonance Imaging

Magnetic resonance imaging scans need to be reviewed locally for any abnormalities. In case of clinically relevant findings, relevant information needs to be provided to the Investigator for appropriate safety reporting and also to ensure the appropriate management of the participant's identified safety finding. When available, a diagnosis of pathology as a cause of such MRI findings or the findings themselves will be reported as an AE until the diagnosis is clear.

### Example 2.6C3—Adverse Event of Special Interest

An AE of special interest (AESI) is an AE (serious or nonserious) of scientific and medical concern specific to the Sponsor's product or program, for which ongoing monitoring and immediate notification by the Investigator to the Sponsor is required. Such events may require further investigation in order to characterize and understand them. Adverse events of special interest may be added, modified, or removed during a study by protocol amendment.

Pregnancy of a female participant entered in a study as well as pregnancy occurring in a female partner of a male participant entered in a study with IMP/NIMP.
It will be qualified as an SAE only if it fulfills one of the seriousness criteria (see Example 2.8B).
In the event of pregnancy in a female participant, the IMP should be discontinued.
Follow up of the pregnancy in a female participant or in a female partner of a male participant is mandatory until the outcome has been determined.

Symptomatic overdose (serious or nonserious) with IMP.
An overdose (accidental or intentional) with the IMP is an event suspected by the Investigator or spontaneously notified by the participant (not based on systematic pills count) and defined as at least twice the intended dose within the intended therapeutic interval (eg, >2 tablets of the IMP within a 12-hour interval).

Increase in alanine transaminase (ALT)>3×ULN.
ALT increase>3×ULN confirmed by retest within 72 hours or in the absence of a retest within 72 hours.

Project specific AESI(s) are:
ECG observation of atrial fibrillation or atrial flutter
Severe infection (Grade 3 or above by National Cancer Institute Common Terminology Criteria for Adverse Event [NCI CTCAE]), that may or may not meet seriousness criteria (eg, a Grade 3 opportunistic infection).
Moderate or severe hemorrhagic events (NCI CTCAE Grade 2 or above), including but not limited to symptomatic bleeding in a critical area or organ such as the CNS, or intraocular bleeding.
Thrombocytopenia, platelet count<75,000/mm³ (see Example 2.8C for the management flow chart).

The definitions of an AE or SAE can be found in Example 2.8B.

Adverse events will be reported by the participant (or, when appropriate, by a caregiver, surrogate, or the participant's legally authorized representative).

The Investigator and any qualified designees are responsible for detecting, documenting, and recording events that meet the definition of an AE or SAE and remain responsible for following up AEs that are serious, considered related to the study intervention or study procedures, or that caused the participant to discontinue the study intervention.

### Example 2.6D—Biomarkers

Blood samples for biomarkers research will be collected from all participants in this study as specified in the SoA (Example 2.1). Participants who have had a relapse during the course of the trial will have the option to collect blood samples for biomarkers from that point on.

Collection of whole blood samples for biomarker research is also part of this study.

Samples will be tested to evaluate biomarkers and their association with the observed clinical responses to tolebrutinib.

In addition, samples will be stored, and analysis may be performed on biomarker variants thought to play a role in MOG antibody disease including, but not limited to, serum analytes to evaluate their association with observed clinical responses to tolebrutinib (in accordance with local regulations).

### Example 2.7—Statistical Considerations

### Example 2.7A—Sample Size Determination

This is an exploratory, single arm study. From clinical experience, it is estimated that MOG antibody disease patients have a relapse once every year on average, meaning that for a given year, about 40% would be expected to be relapse free. For this study, approximately 25 participants will be enrolled to receive study intervention. It would be clinically meaningful to observe at least 65% (n=16) of participants relapse-free after 1 year of tolebrutinib treatment.

### Example 2.7B—Populations for Analyses

The following populations for analyses are defined as shown in Table 17.

TABLE 17
Populations for analyses

| Population | Description |
|---|---|
| Screened | All participants who signed the ICF. |
| Exposed | All screened participants who take at least 1 dose of |
| | study intervention. |
| Enrolled | All participants from screened population who have |
| | been allocated to the intervention regardless of |
| | whether the intervention was received or not. |
| Per | All enrolled and treated participants who complete |
| protocol | 1 year tolebrutinib treatment or experience relapse. |
| (PP) | Participants who prematurely discontinue study |
| | treatment for reasons other than relapse will be |
| | excluded. This will be the primary population used |
| | for efficacy. |
| Safety | All enrolled participants who take at least 1 dose |
| | of study intervention. This will be the population |
| | used for safety and also as a secondary population |
| | for efficacy. |

Participants exposed to study intervention before or without being enrolled will not be considered enrolled and will not be included in any analysis population. The safety experience of these participants will be reported separately.

For any participant enrolled more than once, only the data associated with the first enrollment will be used in any analysis population. The safety experience associated with any later enrollment will be reported separately.

### Example 2.7C—Statistical Analyses

The statistical analysis plan (SAP) will include a more technical and detailed description of the statistical analyses described in this section. This section is a summary of the planned statistical analyses of the most important endpoints including primary and key secondary endpoints. Given this is a single arm efficacy trial, the majority of the data will be expressed as descriptive statistics.

### Example 2.7C1—General Considerations

The baseline value is defined as the last available value before the first dose of IMP. For participants enrolled but not treated, the baseline value is defined as the last available value before enrollment. Baseline EDSS will be calculated as the average of the screening and day 1 assessment values.

The observation period will be divided into 3 segments:
The pre-treatment period is defined as the period from signed ICF up to first IMP administration.
The treatment-emergent (TE) period is defined as the period from the first IMP administration to the last IMP administration+10 days.
The post-treatment period is defined as the period from the end of the treatment-emergent period to the last study assessment for the participant.

### Example 2.7C2—Primary Endpoint(s)

The point estimate and exact 95% confidence interval for the proportion of participants free of relapse at 1 year in the PP population will be provided for evaluation as primary endpoint. The observed success rate will aid in the decision to continue development of tolebrutinib in MOG antibody disease or not.

Additional analyses of relapses will include calculation of time to relapse after enrollment. Kaplan-Meier (KM) plot of the cumulative incidence rate will be provided to depict the course of onset of relapse over time in the PP population. The proportion of participants with relapse at 3, 6, 9 and 12 months will be calculated using the KM estimates. Participants who complete treatment without relapse will be censored at their end of treatment date.

Sensitivity analyses including participants that prematurely discontinue study treatment for reasons other than relapse will be provided where:
All these participants are censored at their date of premature treatment discontinuation.
Participants, if any, who experience relapse in the 10 days after premature discontinuation of tolebrutinib are included as having an event and the others are censored at their date of premature treatment discontinuation+10 days.

Time to relapse on tolebrutinib compared to time to relapse in the same group of participants in the year after initiation of their first treatment for MOG antibody disease will be explored, though those prior treatments will be variable. Furthermore, time to relapse on tolebrutinib compared to time to relapse in the year after first treatment initiation for MOG antibody disease in a propensity score matched external control arm will also be investigated. Crude KM curves and estimates will be provided. Various statistical methods will be explored to provide estimates of the reduction in the probability of a relapse on tolebrutinib compared to “other treatment.” Details will be provided in the SAP.

### Example 2.7C3—Secondary Endpoint(s)

EDSS and MOG antibody titers change from baseline after 1 year of tolebrutinib treatment will be summarized for secondary efficacy endpoints in the PP population without relapse.

### Example 2.7C4—Tertiary/Exploratory Endpoint(s)

Details for any tertiary/exploratory endpoints will be included in the SAP.

### Example 2.7C5—Safety Analysis

All safety analyses will be performed on the Safety Population.

### Example 2.7C6—Adverse Events

General common rules for adverse events: AE summaries will be provided depending on the number of events. In case of small numbers, listings of events will be provided.

The AEs will be analyzed in the following 3 categories:
Pre-treatment AEs: AEs that developed, worsened or became serious during the pre-treatment period.
TEAEs: AEs that developed, worsened or became serious during the treatment-emergent period.
Post-treatment AEs: AEs that developed, worsened or became serious during the post-treatment period.

Information for any deaths will be listed.

Analysis of all adverse events: Adverse event incidence table will be provided for all types of TEAEs: all TEAEs, all treatment emergent AESI (defined with a PT or a prespecified grouping), all treatment emergent SAEs and all TEAEs leading to permanent treatment discontinuation.

The AE summaries will be generated with number (%) of participants experiencing at least one event.

Deaths will also be analyzed.

### Example 2.7C7—Laboratory Variables, Vital Signs and Electrocardiograms (ECGs)

Quantitative analyses: For laboratory variables, vital signs and ECG variables, descriptive statistics for results and changes from baseline will be provided for each planned visit during the on-treatment period. These analyses will be performed using central measurements for MOG antibody titers and other laboratory variables and local measurements for vital signs, and ECG variables.

Analyses of laboratory, ECG and vital signs abnormalities: Potentially clinically significant laboratory, ECG and vital signs abnormalities will be summarized.

### Example 2.7C8—Other Analysis

Biomarker exploratory analyses that will be included in the study report will be described in the SAP.

### Example 2.7C9—Interim Analyses

There is no formal interim analysis planned for this study.

### Example 2.8—Supporting Documentation and Operational Considerations

### Example 2.8A—Clinical Laboratory Tests

The tests detailed in the table below in Table 18 will be performed by the central laboratory, when feasible. Local laboratory results are only required in the event that the central laboratory results are not available in time for either study intervention administration and/or response evaluation. Additionally, if the local laboratory results are used to make either a study intervention decision or response evaluation, the results must be entered into the eCRF.

Protocol-specific requirements for inclusion or exclusion of participants are detailed in Tables 13 and 14 of the protocol.

Additional tests may be performed at any time during the study as determined necessary by the Investigator or required by local regulations. Additional serum or urine pregnancy tests may be performed, as determined necessary by the Investigator or required by local regulation, to establish the absence of pregnancy at any time during the subject's participation in the study.

TABLE 18
Protocol-required laboratory assessments

| Laboratory | | | |
|---|---|---|---|
| assessments | Parameters | | |
| Hematology | Platelet count | RBC indices: | WBC count with differential: |
| | RBC count | MCV | Neutrophils |
| | Hemoglobin | MCH | Lymphocytes |
| | Hematocrit | % Reticulocytes | Monocytes |
| | | | Eosinophils |
| | | | Basophils |
| Clinical chemistry^{a} | BUN | Sodium | AST |
| | Creatinine | Calcium | ALT |
| | Glucose | Total and direct bilirubin | Alkaline phosphatase |
| | Potassium | Total protein | Albumin |
| | | Chloride | Creatine phosphokinase |
| | | Bicarbonate | Lipase (screening only) |

| | |
|---|---|
| Routine | Specific gravity |
| urinalysis | pH, glucose, protein, blood, ketones, bilirubin, |
| | urobilinogen, nitrite, leukocyte esterase by dipstick |
| | Microscopic examination (if blood or protein is |
| | abnormal and for signs of infection) |
| Other | Serum anti-aqauaporin 4 antibody |
| screening | Serum anti-MOG antibody (screening and M12, if no |
| tests | relapses occur during the course of the trial) |
| | FSH (if needed, only in female participants to |
| | confirm postmenopausal state) |
| | Highly sensitive serum or urine β-hCG pregnancy test (as |
| | needed for women of childbearing potential)^{b} |
| | Coagulation: PT/ INR, aPTT |
| | Serology tests for hepatitis B (HBs Ag, anti-HBc IGM and total, |
| | anti-HBs) and C virus (anti-HCV); in case these results are |
| | inconclusive (eg anti-HBs negative and anti-HBc positive or |
| | anti-HC IgG positive), HBV-DNA or HCV-RNA testing, |
| | respectively, should be performed for confirmation. HIV and |
| | other infectious diseases, if locally required. |
| | Tuberculosis test: Blood testing (eg, QuantiFERON ® TB Gold |
| | test) is preferred; skin testing (eg, tuberculin skin test) with |
| | ancillary testing will be allowed if blood testing is not available. |
| | T-SPOT can also be performed, if available.^{c} |

ALT: alanine aminotransferase;
anti-HBc; antibody to hepatitis B core antigen;
anti-HBs: hepatitis B surface antibody;
aPTT: activated partial thromboplastin time;
AST: aspartate aminotransferase;
BUN: blood urea nitrogen;
β-hCG: human chorionic gonadotropin;
FSH; follicle-stimulating hormone;
IEC: independent ethics committee;
INR: international normalized ratio;
HBsAg: hepatitis B surface antigen;
HBV: hepatitis B virus;
HCV: hepatitis C virus;
HIV: human immunodeficiency virus;
Ig: immunoglobulin;
IRB: institutional review board;
MCH: mean corpuscular hemoglobin;
MCV: mean corpuscular volume;
PT: prothrombin time;
RBC: red blood cell;
SGOT: serum glutamic-oxaloacetic transaminase;
SGPT: serum glutamic-pyruvic transaminase;
TB: tuberculosis;
ULN: upper limit of normal;
WBC: white blood cell
^{a}Details of liver chemistry stopping criteria and required actions and follow-up assessments after observations of ALT >3 × ULN are given in Example 1.8C. Clinical laboratory findings of ALT >3 × ULN and bilirubin ≥2 × ULN (>35% direct bilirubin), or ALT >3 × ULN and international normalized ratio (INR) >1.5, if INR measured, that may suggest severe liver injury and must be reported as an SAE.
^{b}Local urine testing for pregnancy will be standard for the protocol (except for the Screening Visit, when a serum pregnancy test is required) unless serum testing is required by local regulation or IRB/IEC.
^{c}Further details are provided in Table 14, E 01.

Biomarkers (blood/serum): neurofilament light chain and glial fibrillary acidic protein level. Optional for participants who have relapsed during the course of the trial.

Investigators must document their review of each laboratory safety report.

### Example 2.8B—AEs and SAEs: Definitions and Procedures for Recording, Evaluating, Follow-Up, and Reporting

### Example 2.8B1—Definition of AE

### Example 2.8B1a—AE Definition

An AE is any untoward medical occurrence in a patient or clinical study participant, temporally associated with the use of study intervention, whether or not considered related to the study intervention.

NOTE: An AE can therefore be any unfavorable and unintended sign (including an abnormal laboratory finding), symptom, or disease (new or exacerbated) temporally associated with the use of study intervention.

### Example 2.8B1b—Events Meeting the AE Definition

Any abnormal laboratory test results (hematology, clinical chemistry, or urinalysis) or other safety assessments (eg, ECG, radiological scans, vital signs measurements), including those that worsen from baseline, considered clinically significant in the medical and scientific judgment of the Investigator (i.e., not related to progression of underlying disease), e.g.:
Leading to IMP discontinuation or modification of dosing, or
Fulfilling a seriousness criterion, or
Defined as an AESI.

Exacerbation of a chronic or intermittent pre-existing condition including either an increase in frequency or intensity of the condition.

New conditions detected or diagnosed after study intervention administration even though it may have been present before the start of the study.

Signs, symptoms, or the clinical sequelae of a suspected drug-drug interaction.

Signs, symptoms, or the clinical sequelae of a suspected overdose of either study intervention or a concomitant medication.

“Lack of efficacy” or “failure of expected pharmacological action” per se will not be reported as an AE or SAE. Such instances will be captured in the efficacy assessments. However, the signs, symptoms, or clinical sequelae resulting from lack of efficacy will be reported as AE or SAE if they fulfill the definition of an AE or SAE.

### Example 2.8B1c—Events NOT Meeting the AE Definition

Any clinically significant abnormal laboratory findings or other abnormal safety assessments which are associated with the underlying disease, unless judged by the Investigator to be more severe than expected for the participant's condition.

The disease/disorder being studied or expected progression, signs, or symptoms of the disease/disorder being studied, unless more severe than expected for the participant's condition.

Medical or surgical procedure (e.g., endoscopy, appendectomy): the condition that leads to the procedure is the AE.

Situations in which an untoward medical occurrence did not occur (social or convenience admission to a hospital).

Anticipated day-to-day fluctuations of pre-existing disease(s) or condition(s) present or detected at the start of the study that do not worsen.

### Example 2.8B2—Definition of SAE

An SAE is defined as any adverse event that, at any dose which results in the following.

Results in death.

Is life-threatening: The term “life-threatening” in the definition of “serious” refers to an event in which the participant was at risk of death at the time of the event. It does not refer to an event, which hypothetically might have caused death, if it were more severe.

Requires inpatient hospitalization or prolongation of existing hospitalization: In general, hospitalization signifies that the participant has been admitted (usually involving at least an overnight stay) at the hospital or emergency ward for observation or treatment that would not have been appropriate in the physician's office or outpatient setting. Complications that occur during hospitalization are AEs. If a complication prolongs hospitalization or fulfills any other serious criteria, the event is serious. When in doubt as to whether “hospitalization” occurred or was necessary, the AE should be considered serious. Hospitalization for elective treatment of a pre-existing condition that did not worsen from baseline is not considered an AE.

Results in persistent or significant disability/incapacity.
The term disability means a substantial disruption of a person's ability to conduct normal life functions.
This definition is not intended to include experiences of relatively minor medical significance such as uncomplicated headache, nausea, vomiting, diarrhea, influenza, and accidental trauma (eg, sprained ankle) which may interfere with or prevent everyday life functions but do not constitute a substantial disruption.

Is a congenital anomaly/birth defect.

Other situations:
Medical or scientific judgment should be exercised by the Investigator in deciding whether SAE reporting is appropriate in other situations such as significant medical events that may jeopardize the participant or may require medical or surgical intervention to prevent one of the other outcomes listed in the above definition. These events should usually be considered serious.
Note: The following list of medically important events is intended to serve as a guideline for determining which condition has to be considered as a medically important event. The list is not intended to be exhaustive.
Intensive treatment in an emergency room or at home for:
   Allergic bronchospasm.
   Blood dyscrasias (i.e., agranulocytosis, aplastic anemia, bone marrow aplasia, myelodysplasia, pancytopenia, etc).
   Convulsions (seizures, epilepsy, epileptic fit, absence, etc).
Development of drug dependence or drug abuse.
ALT>3×ULN+total bilirubin>2×ULN or asymptomatic ALT increase>10×ULN.
Suicide attempt or any event suggestive of suicidality.
Syncope, loss of consciousness (except if documented as a consequence of blood sampling).
Bullous cutaneous eruptions.
Cancers diagnosed during the study or aggravated during the study.

### Example 2.8B3—Assessment and Follow-Up of AE and/or SAE

### Example 2.8B3a—Assessment of Severity

The Investigator will assess the severity for each AE and SAE using the National Cancer Institute Common Terminology Criteria for Adverse Events (NCI CTCAE) version 5.0, published on 27 Nov. 2017. Listings of MedDRA terms should be consulted first in NCI CTCAE to look for severity grade description for a particular AE. For AEs not listed in the NCI CTCAE, the Investigator will be required to assess the severity of the AE using general guideline:
Grade 1 Mild; asymptomatic or mild symptoms; clinical or diagnostic observations only; intervention not indicated.
Grade 2 Moderate; minimal, local or noninvasive intervention indicated; limiting age-appropriate instrumental ADL*.
Grade 3 Severe or medically significant but not immediately life-threatening; hospitalization or prolongation of hospitalization indicated; disabling; limiting self-care ADL**.
Grade 4 Life-threatening consequences; urgent intervention indicated.
Grade 5 Death related to AE.

### Note: Activities of Daily Living (ADL)

*Instrumental ADL refer to preparing meals, shopping for groceries or clothes, using the telephone, managing money, etc.
**Self-care ADL refer to bathing, dressing and undressing, feeding self, using the toilet, taking medications, and not bedridden.

Any Grade 4 or 5 event must be reported as an SAE. A Grade 1 through 3 event is defined as ‘serious’ when it meets at least 1 of the predefined outcomes as described in the definition of an SAE.

### Example 2.8B3b—Assessment of Causality

The Investigator is obligated to assess the relationship between study intervention and each occurrence of each AE/SAE.

A “reasonable possibility” of a relationship conveys that there are facts, evidence, or arguments to suggest a causal relationship, rather than that a relationship cannot be ruled out.

The Investigator will use clinical judgment to determine the relationship.

Alternative causes, such as underlying disease(s), concomitant therapy, and other risk factors, as well as the temporal relationship of the event to study intervention administration will be considered and investigated.

The Investigator will also consult the Investigator's Brochure (IB) or Product Information, for marketed products, in his/her assessment.

For each AE/SAE, the Investigator must document in the medical notes that he/she has reviewed the AE/SAE and has provided an assessment of causality.

The Investigator may change his/her opinion of causality in light of follow-up information and send an SAE follow-up report with the updated causality assessment.

The causality assessment is one of the criteria used when determining regulatory reporting requirements.

### Example 2.8B3c—Follow-Up of AEs and SAEs

The Investigator is obligated to perform or arrange for the conduct of supplemental measurements or evaluations as medically indicated to elucidate the nature or causality of the AE or SAE as fully as possible. This may include additional laboratory tests or investigations, histopathological examinations, or consultation with other health care professionals.

### Example 2.8C—Liver and Other Safety: Suggested Actions and Follow-Up Assessments

These actions described in Table 19 and FIGS. 2, 3, 4B, 5, and 6 are required for ALT increase and thrombocytopenia events ONLY. For all other safety events described, these are suggested per the Investigator's medical judgement.

Thrombocytopenia is to be recorded as an AE only if at least 1 of the criteria listed in the general guidelines for reporting adverse events in Example 2.8B is met (FIG. 3). Abbreviations in FIG. 3: aPTT, activated partial thromboplastin time; EDTA, Ethylenediaminetetraacetic acid; INR, international normalized ratio; PK, pharmacokinetic(s); PT, prothrombin time.

Neutropenia is to be recorded as an AE only if at least 1 of the criteria listed in the general guidelines for reporting adverse events in Example 2.8B is met.

Abbreviations in FIG. 4B: ALT: alanine aminotransferase; AST: aspartate aminotransferase; CMV: cytomegalovirus; CPK: creatine phosphokinase; CRF: case report form; EBV: Epstein-Barr virus; HAV: hepatitis A virus; HBV: hepatitis B virus; HCV: hepatitis C virus; IgM: immunoglobulin M; IMP: investigational medicinal product; INR: international normalized ratio; LFT: liver function test; LKM: liver-kidney microsomal antibody; PT: prothrombin time; ULN: upper limit of normal.

Note: In FIG. 4B, “Baseline” refers to ALT sampled at baseline visit; or if baseline value unavailable, to the latest ALT sampled before the baseline visit. The algorithm does not apply to the instances of increase in ALT during screening.

In FIG. 4B, Normalization is defined as ≤ULN or baseline value, if baseline value is >ULN.

Increase in serum creatinine is to be recorded as an AE only if at least 1 of the criteria listed in the general guidelines for reporting adverse events in Example 1.8B is met (FIG. 5).

Abbreviations in FIG. 5: ARF, acute renal failure; ULN, upper limit of normal; DIC, disseminated intravascular coagulation; CPK, creatine phosphokinase; ECG, electrocardiogram; PK, pharmacokinetic(s).

Increase in CPK is to be recorded as an AE only if at least 1 of the criteria in the general guidelines for reporting adverse events in Example 2.8B is met.

If either the clinical presentation or MRI features of a participant are suggestive of PML, the following diagnostic and action algorithm is recommended (FIG. 6).

Abbreviations in FIG. 6: CSF: cerebrospinal fluid; Gd: gadolinium; IMP: investigational medicinal product; JCV: John Cunningham virus; MRI: magnetic resonance imaging; PCR: polymerase chain reaction; PML: progressive multifocal leukoencephalopathy.

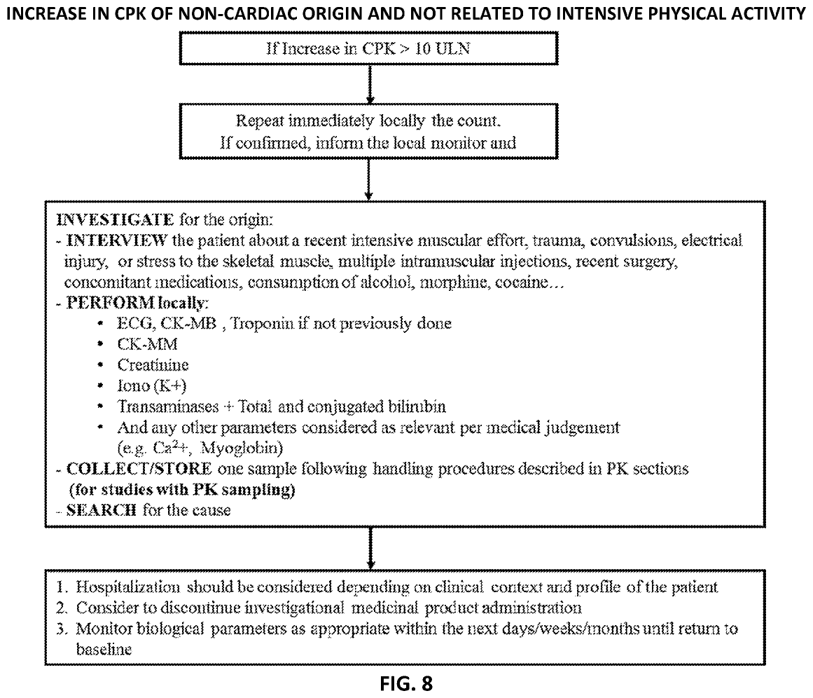

Abbreviations in FIG. 8: CK-MB, creatine kinase-MB; CK-MM, creatine kinase-MM; ECG, electrocardiogram; PK, pharmacokinetic(s); ULN, upper limit of normal.

Clinical manifestations or MRI lesions features suspicious for PML are proposed in Table 19.

TABLE 19
Clinical and MRI features suggestive of PML

| | |
|---|---|
| Clinical | Subacute onset of weakness, sensory deficits, cognitive |
| history | or behavioral abnormalities, gait dysfunction, speech/ |
| | language difficulties or any other signs of cortical |
| | dysfunction, retrochiasmal visual defects or seizure |
| Brain | ≥1 T2/FLAIR hyperintense and T1 hypointense lesions |
| MRI | involving the subcortical and juxtacortical white |
| | matter, sparing the cortex, with no mass effect, with a |
| | continuous progression; new lesions with no enhancement |
| | (even when large) or with faint rim enhancement |

In the event that PML is suspected based on imaging results, the local radiologist will directly inform the Investigator. The Investigator will obtain additional plasma, urine, and CSF samples for John Cunningham virus (JCV) analysis. Samples will be analyzed upon receipt and the results will be provided directly to the investigational site and to the Sponsor. Further management will be deferred to the Treating Investigator. However, next steps will include discontinuation of study treatment. Additional imaging will be at the discretion of the Investigator depending on the diagnostic workup and treatment plan.

The detection of JCV DNA in the CSF of a patient with clinical and MRI features suggestive of PML establishes the diagnosis of PML.

If JCV DNA is not detected in CSF and if clinical suspicion of PML remains high, another lumbar puncture should be performed.

If diagnosis remains uncertain and suspicion of PML remains high, a brain biopsy may be considered to establish a definitive diagnosis.

Clinical or MRI features suggestive of PML should be recorded as an AE/AESI/SAE following the definitions and procedures in Example 1.8B.

TABLE 20
Actions for cases of confirmed ALT elevation

In ANY CONFIRMED CASE of ALT >5 × ULN, the following steps are
REQUIRED (recommended for ALT >3 × ULN but ALT <5 × ULN, as clinically
indicated):
INFORM the Site Monitor, who will forward the information to the Study
Manager.
INVESTIGATE specifically for malaise with or without loss of
consciousness, dizziness, and/or hypotension and/or episode of
arrhythmia since the last visit, particularly in the previous 72 hours;
rule out muscular injury.
PERFORM the following tests/actions:
LFTs: AST, ALT, alkaline phosphatase, total and conjugated
bilirubin and prothrombin time/INR (mandatory assessments
for ALT >3 × ULN);
CPK, serum creatinine, complete blood count;
Anti-HAV IgM, anti-HBc IgM, (HBV-DNA if clinically
indicated), anti-HCV and HCV RNA, anti-CMV IgM and anti-
HEV IgM antibodies;
Iron, ferritin, transferrin saturation;
Auto-antibodies: antinuclear, anti-DNA, anti-smooth muscle, anti-LKM,
anti-mitochondrial;
Evaluate recent infection with EBV, herpes viruses. Depending on the
clinical context, consider testing for toxoplasma;
Collect and freeze serum sample (5 mL × 2);
Collect and store one PK sample following the instructions in the central
laboratory manual;
Consider hepatobiliary ultrasonography (or other imaging investigations
if needed);
Consider DNA test for Gilbert's disease if clinically indicated;
Consider consulting with a hepatologist (mandatory if ALT >8 × ULN or
is associated with elevated bilirubin);
Discuss clinical indication for potential liver biopsy with the
hepatologist;
Consider patient hospitalization if INR >2 (or PT <50%) and/or central
nervous system disturbances suggesting hepatic encephalopathy
MONITOR LFTs after discontinuation of IMP:
Monitor closely (every 2-3 days) until ALT is down-trending, then
weekly until <1.5 × ULN, and then at everyscheduled visit;
This frequent LFT monitoring may be done through central or local lab,
or via home visit (depending on the Investigator's assessment and/or
local regulatory requirements).
Rechallenge: Re-initiation of the study drug can only be considered
after discussion with the Sponsor's Medical Monitor once the ALT/AST
decreases to <1.5 × ULN, and there is no clinical contraindication. No
rechallenge will be considered for participants with >3 × ULN ALT
and >2 × ULN bilirubin increase.
In case it is agreed to re-start the study drug, it is recommended
that ALT/AST be assessed per protocol schedule of assessments
for the first 6 months of the treatment period.
The occurrence of new elevation >3 × ULN for the ALT/AST
values will lead to permanent discontinuation of the study drug.

### Example 2.8D—Diagnostic Criteria for MOG Antibody Disease

All of the following criteria must be met for the definitive diagnosis of MOG antibody disease (Jarius et al. J Neuroinflammation 2018, 15, 134):
Monophasic or relapsing acute ON, myelitis, brainstem encephalitis, or encephalitis, or any combination of these syndromes.
MRI or electrophysiological (visual evoked potentials in patients with isolated ON) findings compatible with CNS demyelination.
Seropositivity for MOG-IgG as detected by means of a cell-based assay employing full-length human MOG as target antigen.

### Example 1.8E—Abbreviations

Ab: antibody
ADL: activities of daily living
AE: adverse event
AESI: adverse event of special interest
ALT: alanine aminotransferase
aPTT: activated partial thromboplastin time
ARF: acute renal failure
ARR: annualized relapse rate
BTK: Bruton's tyrosine kinase
CDP: confirmed disability progression
CK-MB: creatine kinase (heart)
CK-MM: creatine kinase (skeletal muscle)
CNS: central nervous system
CPK: creatinine phosphokinase
CRF: case report form
CSF: cerebrospinal fluid
CSR: clinical study report
C-SSRS: Columbia Suicide Severity Rating Scale
CYP cytochrome P450
DMC: Data Monitoring Committee
DMT: disease-modifying therapies
DNA: deoxyribonucleic acid
DTP: direct to patient
ECG: electrocardiogram, electrocardiography
eCRF: electronic case report form
EDSS: expanded disability status scale
eGFR: estimated glomerular filtration rate
EOS: end of study
EOT: end of treatment
FSH: follicle stimulating hormone
GCIPL: ganglion cell-inner plexiform layer
GCP: good clinical practice
Gd: gadolinium
HIV: human immunodeficiency virus
HR: hazard ratio
HRT: hormone replacement therapy
IB: Investigators brochure
ICF: informed consent form
ICH: International Council for Harmonisation
IEC: independent ethics committees
Ig: immunoglobulin
IL: interleukin
IMP: investigational medicinal product
INR: international normalized ratio
IRB: institutional review board
ITT: intent-to-treat
IUD: intrauterine device
IUS: intrauterine hormone-releasing system
IV: intravenous
IVIG: intravenous immunoglobulins
JCV: John Cunningham virus
KM: Kaplan-Meier
LFT: liver function tests
LLN: lower limit of normal
MedDRA: Medical Dictionary for Regulatory Activities
MOG: myelin oligodendrocyte glycoprotein
MOGAD: myelin oligodendrocyte glycoprotein antibody-associated-disease
MRI: magnetic resonance imaging
MS: multiple sclerosis
NCI CTCAE: National Cancer Institute Common Terminology Criteria for Adverse Event
NfL: neurofilament light chain
NIMP: noninvestigational medicinal product
NMO: neuromyelitis optica
NSAIDs: nonsteroidal anti-inflammatory drugs
NYHA: New York Heart Association
OCT: optical computerized tomography
ON: optic neuritis
PBPK: physiologically based pharmacokinetic
PD: pharmacodynamic
PK: pharmacokinetic(s)
PML: progressive multifocal leukoencephalopathy
PP: per protocol
PT: prothrombin time
QTcF: QT interval corrected using Fridericia's formula
RMS: relapsing multiple sclerosis
RNFL: retinal nerve fiber layer
SAE: serious adverse event
SAP: statistical analysis plan
SoA: schedule of activities
SUSAR: suspected unexpected serious adverse reaction
TB: tuberculosis
TEAE: treatment-emergent adverse event
US: United States
WOCBP: woman of childbearing potential

1. A method of treating myelin oligodendrocyte glycoprotein antibody disease (MOGAD), comprising administering to a subject in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one or a pharmaceutically acceptable salt thereof.

2. A method of reducing the frequency of myelin oligodendrocyte glycoprotein antibody disease (MOGAD) relapse in a subject having MOGAD, comprising administering to a subject in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one or a pharmaceutically acceptable salt thereof.

3. A method of reducing MOG antibody titers in a subject having myelin oligodendrocyte glycoprotein antibody disease (MOGAD), comprising administering to a subject in need thereof a therapeutically effective amount of a BTK inhibitor comprising (R)-1-(1-acryloylpiperidin-3-yl)-4-amino-3-(4-phenoxyphenyl)-1H-imidazo[4,5-c]pyridin-2(3H)-one or a pharmaceutically acceptable salt thereof.

4. The method of claim 1, wherein the subject has at least one symptom of myelin oligodendrocyte glycoprotein antibody disease (MOGAD) prior to treatment.

5. The method of claim 4, wherein the at least one myelin oligodendrocyte glycoprotein antibody disease (MOGAD) symptom is chosen from loss of color vision, loss or blurring of vision, paralysis/paraparesis of limbs, and loss of bladder/bowel control.

6. The method of claim 5, wherein the treatment reduces or eliminates at least one symptom of myelin oligodendrocyte glycoprotein antibody disease (MOGAD).

7. The method of claim 1, wherein the subject is relapse-free about 1 year.

8. The method of claim 1, wherein the subject has at least one confirmed relapse in the previous 12 months or 2 confirmed relapses in the previous 24 months at screening.

9. The method of claim 8, wherein the subject in relapse is administered a corticosteroid.

10. The method of claim 9, wherein the corticosteroid is methylprednisone.

11. The method of claim 1, wherein a dose of about 5 mg to about 60 mg of the BTK inhibitor is administered.

12. (canceled)

13. (canceled)

14. (canceled)

15. (canceled)

16. The method of claim 1, wherein the dose is once daily.

17. The method of claim 1, wherein the dose is administered once daily with food.

18. The method of claim 1, wherein the dose is 60 mg and is administered once daily with food.

19. The method of claim 1, wherein the BTK inhibitor compound is administered as monotherapy.

20. The method of claim 1, wherein myelin oligodendrocyte glycoprotein antibody disease (MOGAD) is chosen from acquired demyelinating syndromes (ADS).

21. The method of claim 20, wherein the acquired demyelinating syndrome (ADS) is acute disseminated encephalomyelitis (ADEM).

22. The method of claim 1, wherein the subject is a human.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. A method of treating myelin oligodendrocyte glycoprotein antibody disease MOGAD) in a patient in need thereof, comprising administering a therapeutically effective amount BTK inhibitor comprising (R)-1-(1-acryloylpiperidin-3-yl)-4-amino-3-(4-phenoxyphenyl)-1H-imidazo[4,5-c]pyridin-2(3H)-one or a pharmaceutically acceptable salt thereof to the patient, wherein the patient is not receiving potent and moderate inducers of cytochrome P450 3A (CYP3A) or potent inhibitors of CYP2C8 hepatic enzymes.

39. A method of treating myelin oligodendrocyte glycoprotein antibody disease MOGAD) in a patient in need thereof, comprising the steps of:
(a) advising the patient to limit alcohol consumption during treatment; and
(b) administering a therapeutically effective amount BTK inhibitor comprising (R)-1-(1-acryloylpiperidin-3-yl)-4-amino-3-(4-phenoxyphenyl)-1H-imidazo[4,5-c]pyridin-2(3H)-one or a pharmaceutically acceptable salt thereof to the patient,
wherein the patient is female and is advised to limit alcohol consumption to 14 grams/day or less, or the patient is male and is advised to limit alcohol consumption to 28 grams/day or less.
